(19) United States
(12) Patent Application Publication
(10) Pub. No.: US 2026/0324628 A1
(43) Pub. Date: Oct. 22, 2026

(54) HYDROPHOBIC AND HYDROPHILIC MODIFIED MALEATED NATURAL OILS, SALTS AND ITS AGRICULTURAL COMPOSITIONS AND METHODS OF USE

(71) Applicant: ISP INVESTMENTS LLC, Wilmington, DE (US)

(72) Inventors: Osama M. MUSA, Bedminster, NJ (US); Henry Keith CHENAULT, North Plainfield, NJ (US); Kiel Trenton MOORE, Honesdale, PA (US); Mousumi GHOSH, Elmwood Park, NJ (US); Drupesh PATEL, Lake Hiawatha, NJ (US)

(73) Assignee: ISP INVESTMENTS LLC, Wilmington, DE (US)

(21) Appl. No.: 19/480,277

(22) PCT Filed: May 2, 2024
(86) PCT No.: PCT/US2024/027353

§ 371 Date: Oct. 30, 2025

Related U.S. Application Data
(60) Provisional application No. 63/463,454, filed on May 2, 2023.

(51) Int. Cl. A01N 25/04; A01N 43/653; A01N 43/70; A01N 47/36; A01N 51/00; A01N 53/00; A01N 57/16; A01P 3/00; A01P 7/04; A01P 13/00; C07C 69/73; C07D 307/60

(52) U.S. Cl. CPC A01N 25/04; A01N 43/653; A01N 43/70; A01N 47/36; A01N 51/00; A01N 53/00; A01N 57/16; A01P 3/00; A01P 7/04; A01P 13/00; C07C 69/73; C07D 307/60

(57) ABSTRACT

The present invention provides agricultural compositions comprising a reaction product of (a) a maleated natural oil, comprising a natural oil with maleated functionality; and (b) a base. The present invention also provides agricultural compositions comprising a reaction product of (a) a maleated natural oil with maleated functionality; (b) a base; and (c) a functionalized or unfunctionalized moiety selected from the group consisting of hydrophobic moieties, hydrophilic moieties, and combinations thereof.

Additionally, the present invention provides compositions comprising a reaction product of (a) a maleated natural oil with maleated functionality; (b) a functionalized or unfunctionalized moiety selected from the group consisting of hydrophobic moieties, hydrophilic moieties, and combinations thereof and (c) a base to form a reaction product of structure (I) or (II):

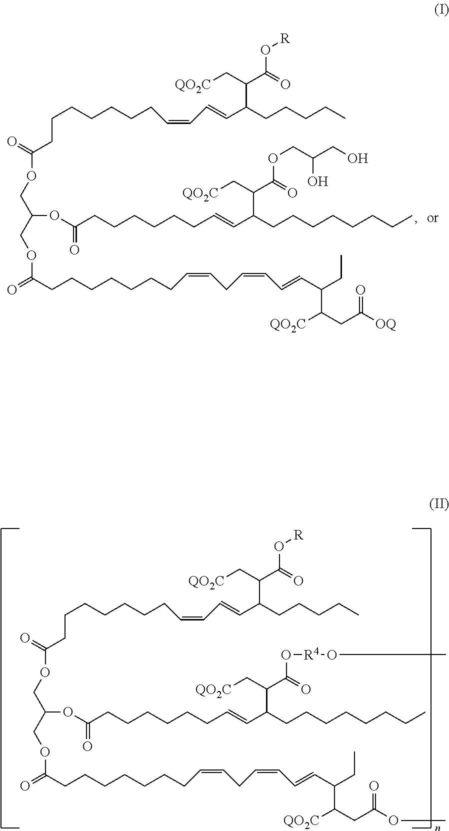

wherein 1<n<30; wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

### FIELD OF THE INVENTION

The present application provides an agricultural composition comprising modified maleated natural oils. The modified maleated natural oils do not exhibit many of the limited properties of maleated natural oils. The modified maleated natural oils are useful in a wide variety of agricultural compositions and applications.

### BACKGROUND OF THE INVENTION

Modified natural oils can be non-dispersible in water or alcohols. As a result, these oils can be incorporated into a wide variety of compositions. Such compositions include, but are not limited to, personal care (e.g., hair care, sun care, skin care, oral care), adhesives, coatings, paints, electronics, household, industrial and institutional (HI&I) compositions, inks, membranes, metal working fluids, oilfield chemicals, plastics and plasticizers, textiles, industrial products, biocides, pharmaceuticals/nutritionals, and agrochemical compositions.

Natural oils are useful for synthesizing renewable compounds such as polymers, plastics, and plasticizers, which compounds are useful in a variety of agricultural compositions. A difficulty with utilizing natural oils is that they are blends of triglycerides containing varying degrees of unsaturated groups, which unsaturated groups are relatively unreactive. To make these natural oils reactive, these unsaturated groups are generally chemically modified to make them reactive. For example, these unsaturated groups can be reacted to provide epoxide functional groups or succinic anhydride functional groups. Despite these chemical modifications, the maleated natural oils can still exhibit limited properties such as insolubility or non-dispersibility in water and alcohols. Accordingly, there is a need for further modified maleated natural oils which do not exhibit the limited properties of maleated natural oils.

Natural oils are useful for synthesizing renewable compounds such as polymers, plastics, and plasticizers, which compounds are useful in a variety of agricultural compositions. A difficulty with utilizing natural oils is that they are blends of triglycerides containing varying degrees of unsaturated groups, which unsaturated groups are relatively unreactive. To make these natural oils reactive, these unsaturated groups are generally chemically modified to make them reactive. For example, these unsaturated groups can be reacted to provide epoxide functional groups or succinic anhydride functional groups. Despite these chemical modifications, the maleated natural oils can still exhibit limited properties such as insolubility or non-dispersibility in water and alcohols. Accordingly, there is a need for further modified maleated natural oils which do not exhibit the limited properties of maleated natural oils.

Natural oils are useful for synthesizing renewable compounds such as polymers, plastics, and plasticizers, which compounds are useful in a variety of agricultural compositions. A difficulty with utilizing natural oils is that they are blends of triglycerides containing varying degrees of unsaturated groups, which unsaturated groups are relatively unreactive. To make these natural oils reactive, these unsaturated groups are generally chemically modified to make them reactive. For example, these unsaturated groups can be reacted to provide epoxide functional groups or succinic anhydride functional groups. Despite these chemical modifications, the maleated natural oils can still exhibit limited properties such as insolubility or non-dispersibility in water and alcohols. Accordingly, there is a need for further modified maleated natural oils which do not exhibit the limited properties of maleated natural oils.

Natural oils are useful for synthesizing renewable compounds such as polymers, plastics, and plasticizers, which compounds are useful in a variety of agricultural compositions. A difficulty with utilizing natural oils is that they are blends of triglycerides containing varying degrees of unsaturated groups, which unsaturated groups are relatively unreactive. To make these natural oils reactive, these unsaturated groups are generally chemically modified to make them reactive. For example, these unsaturated groups can be reacted to provide epoxide functional groups or succinic anhydride functional groups. Despite these chemical modifications, the maleated natural oils can still exhibit limited properties such as insolubility or non-dispersibility in water and alcohols. Accordingly, there is a need for further modified maleated natural oils which do not exhibit the limited properties of maleated natural oils.

Natural oils are useful for synthesizing renewable compounds such as polymers, plastics, and plasticizers, which compounds are useful in a variety of agricultural compositions. A difficulty with utilizing natural oils is that they are blends of triglycerides containing varying degrees of unsaturated groups, which unsaturated groups are relatively unreactive. To make these natural oils reactive, these unsaturated groups are generally chemically modified to make them reactive. For example, these unsaturated groups can be reacted to provide epoxide functional groups or succinic anhydride functional groups. Despite these chemical modifications, the maleated natural oils can still exhibit limited properties such as insolubility or non-dispersibility in water and alcohols. Accordingly, there is a need for further modified maleated natural oils which do not exhibit the limited properties of maleated natural oils.

Natural oils are useful for synthesizing renewable compounds such as polymers, plastics, and plasticizers, which compounds are useful in a variety of agricultural compositions. A difficulty with utilizing natural oils is that they are blends of triglycerides containing varying degrees of unsaturated groups, which unsaturated groups are relatively unreactive. To make these natural oils reactive, these unsaturated groups are generally chemically modified to make them reactive. For example, these unsaturated groups can be reacted to provide epoxide functional groups or succinic anhydride functional groups. Despite these chemical modifications, the maleated natural oils can still exhibit limited properties such as insolubility or non-dispersibility in water and alcohols. Accordingly, there is a need for further modified maleated natural oils which do not exhibit the limited properties of maleated natural oils.

Natural oils are useful for synthesizing renewable compounds such as polymers, plastics, and plasticizers, which compounds are useful in a variety of agricultural compositions. A difficulty with utilizing natural oils is that they are blends of triglycerides containing varying degrees of unsaturated groups, which unsaturated groups are relatively unreactive. To make these natural oils reactive, these unsaturated groups are generally chemically modified to make them reactive. For example, these unsaturated groups can be reacted to provide epoxide functional groups or succinic anhydride functional groups. Despite these chemical modifications, the maleated natural oils can still exhibit limited properties such as insolubility or non-dispersibility in water and alcohols. Accordingly, there is a need for further modified maleated natural oils which do not exhibit the limited properties of maleated natural oils.

Natural oils are useful for synthesizing renewable compounds such as polymers, plastics, and plasticizers, which compounds are useful in a variety of agricultural compositions. A difficulty with utilizing natural oils is that they are blends of triglycerides containing varying degrees of unsaturated groups, which unsaturated groups are relatively unreactive. To make these natural oils reactive, these unsaturated groups are generally chemically modified to make them reactive. For example, these unsaturated groups can be reacted to provide epoxide functional groups or succinic anhydride functional groups. Despite these chemical modifications, the maleated natural oils can still exhibit limited properties such as insolubility or non-dispersibility in water and alcohols. Accordingly, there is a need for further modified maleated natural oils which do not exhibit the limited properties of maleated natural oils.

Natural oils are useful for synthesizing renewable compounds such as polymers, plastics, and plasticizers, which compounds are useful in a variety of agricultural compositions. A difficulty with utilizing natural oils is that they are blends of triglycerides containing varying degrees of unsaturated groups, which unsaturated groups are relatively unreactive. To make these natural oils reactive, these unsaturated groups are generally chemically modified to make them reactive. For example, these unsaturated groups can be reacted to provide epoxide functional groups or succinic anhydride functional groups. Despite these chemical modifications, the maleated natural oils can still exhibit limited properties such as insolubility or non-dispersibility in water and alcohols. Accordingly, there is a need for further modified maleated natural oils which do not exhibit the limited properties of maleated natural oils.

Maleated natural oils are natural oils that have been chemically functionalized by the chemical addition of epoxide (oxirane) and succinic anhydride functional groups. Examples include epoxidized and maleated soybean oil and linseed oil, unsaturated natural oils lend themselves to these chemical functionalizations.

U.S. Pat. No. 9,809,538B2 describes a modified natural compound synthesized from epoxidized natural fatty acid, maleated natural fatty acid, epoxidized natural oil, or maleated natural oil; and lactam compound with hydroxyl(s) to form e.g., adhesive or beverage composition.

A discussion of reaction scheme for the maleinization reaction in the vegetable oil is provided in the article “Maleated soybean oil and its multifunctional properties,” by Gripp, Anna A., Steinberg, David C., published in Cosmetics Exhibition & Conference Conference Proceedings, Barcelona, Mar. 22-24, 1994.

A discussion of reaction scheme for the maleinization reaction in the vegetable oil is provided in the article “Microwave Assisted Syntheses of Vegetable Oil Based Monomer,” by Rafael T. Alarcon et al., published in Journal of Polymers and the Environment 28:1265-1278, 2020.

A discussion of generalized reaction between maleic anhydride and unsaturated vegetable oils is provided in the handbook of maleic Anhydride based materials—syntheses, properties and applications by Osama M. Musa in chapter 3 page 166, published in Springer International Publishing Switzerland 2016.

U.S. Pat. No. 2,754,306A describes a reaction with a soybean oil, maleic anhydride based and iso-octyl alcohol to provide an improved plasticizer for nitrocellulose compositions.

PCT Application 2019113068A1 and 2005071050A1 describes a technology related to metalworking fluids comprising maleated soybean oil derivatives.

A discussion of maleic anhydride polymerization and modified plant oils with polyols is provided in the article “Polymerization of Maleic Anhydride-Modified Plant Oils with Polyols,” by Tarik Eren, Selim H. Kusefoglu, Richard Wool published in Journal of Applied Polymer Science, Barcelona, Volume 90, Issue 1, Pages 197-202, 2003.

EP Patent 2754306A describes an adhesive containing a polycondensate and a dienophile modified fatty acid as a cross-linking agent.

US Publication 20180070584A1 describes an adjuvant composition that includes a maleated natural oil derivative into agrochemical formulations and applied to target substrates to kill, inhibit, or repel pests.

PCT Application 2005071050A1 describes a metalworking fluid comprising oil in water emulsion from a reaction product of maleic anhydride and a triglyceride oil from a plant or land animal and further reacted with water, Group IA and IIA metals, ammonium hydroxide, various amines, alkanolamines, polyols, alkoxylated alkanolamines, poly (alkylene oxide)s, or polyamines or mixtures.

U.S. patent Ser. No. 10/889,693 B2 discloses a composition used to treat agricultural plants, comprises modified oil alkyl ester and/or modified oil aryl ester having transesterification reaction product of oil (soybean oil and surfactant having hydroxy group.

U.S. Pat. No. 5,733,970 A discloses an aqueous dispersed, epoxy crosslinked maleated oil (maleated glyceride oil) microgel polymers for protective coatings.

US Publication 20040197363 A1 discloses a coated granular material is selected from the group consisting of fertilizers, crop protection agents, insecticides, pesticides, fungicides, drying agents and mixtures having curing agent in example Maleic Acid Anhydride/Soybean Oil Adduct.

U.S. Pat. No. 9,809,538 B2 discloses a modified natural compound synthesized from epoxidized natural fatty acid, maleated natural fatty acid, epoxidized natural oil, or maleated natural oil; and lactam compound with hydroxyl(s) used in application of agriculture.

US Publication 20130210630 A1 discloses a self-emulsifying oil having vegetable oil modified (maleated soyabean oil) by reacting it with moiety more polar than vegetable oil; and an active ingredient e.g. herbicide.

Despite the renewability, biodegradability, sustainability, and beneficial functions provided by natural fatty acids, natural oils and their maleated counterparts, they exhibit properties that can limit their application. For example, maleated soybean oil is insoluble and non-dispersible in water or alcohols. As a result, these oils may tend to exude or phase-separate from formulated compositions. This feature makes their formulation more difficult, often requiring additional ingredients to facilitate solutions, emulsions, or dispersions. Natural and maleated natural oils may not impart the desired property needed in end-uses, such as solubilization capability, glass transition, flexibility, shine, and/or plasticization. Consequently, the performance (including, but not limited to stability, resistance to phase separation, absorption, clean-up, solubility potential, staining potential, lubrication, film formation, uniformity of spreading, comedogenic tendency, ease of removal), may be less than desired. Finally, although such natural oils are an important renewable material, they are not always the formulator's first choice, and, in fact, often are not considered at all.

Mineral oils and vegetable oils are often used as components of agricultural compositions. Accordingly, there is a need for materials that are renewable, natural, and biodegradable having different and controllable chemical, physical, and/or mechanical properties such that the limitations found in natural and maleated natural oils are minimized or eliminated. Most agricultural compositions contain more than one inert ingredient to help deliver the active ingredient having a fungicide, bactericide, insecticide, and herbicide activity in combination of inert ingredient or an adjuvant used in plant treatments. The employed agricultural composition comprising modified maleated natural oils in combination with agriculturally active ingredients and specific ingredients can improve efficiency of the active ingredient itself and used for agriculture. Therefore, there is a need to develop new, functionalized maleated natural oils for use in curative treatments of the agricultural cultivations and agriculture compositions that are non-toxic and inspired by or created from renewable resources.

### SUMMARY OF THE INVENTION

The present invention provides an agricultural composition comprising a reaction product of (a) a maleated natural oil, comprising a natural oil with maleated functionality; and (b) a base.

In another aspect of the invention, the present application provides an agricultural composition comprising a reaction product of (a) a maleated natural oil, comprising a natural oil with maleated functionality; (b) a base and further comprising one or more agriculturally active ingredient; and one or more additional ingredient.

In another aspect of the invention, the present application provides an agriculture composition comprising (A) from about 0.01 to about 20.0 wt. % of a reaction product of (a) a maleated natural oil, comprising a natural oil with maleated functionality; and (b) a base; and (B) from about 0.1 to about 90.0 wt. % of one or more agriculturally active ingredient; and (C) from about 1 to about 99.0 wt. % of one or more additional ingredients.

Another aspect of the present invention provides an agricultural composition comprising a reaction product of (A) a maleated natural oil, comprising a natural oil with maleated functionality; (B) a base; wherein the maleated functionality has been partially reacted with the base; and (C) a functionalized or unfunctionalized moiety selected from the group consisting of hydrophobic moieties, hydrophilic moieties, and combinations thereof; wherein the maleated functionality has been partially reacted with the functionalized or unfunctionalized moiety.

In an another aspect of the present invention provides an agricultural composition comprising a reaction product of (A) from about 0.01% to about 20.0% of a reaction product of (a) a maleated natural oil; and (b) a base; wherein the maleated functionality has been partially reacted with the base; and (c) a functionalized or unfunctionalized moiety selected from the group consisting of hydrophobic moieties, hydrophilic moieties, and combinations thereof wherein the maleated functionality has been partially reacted with the functionalized or unfunctionalized moiety; and (B) from about 1.0. to about 90.0 wt. % of one or more agriculturally active ingredients; and (C) from about 1 to about 99.0 wt. % of one or more ingredients.

In an another aspect of the present invention provides an composition comprising a reaction product of: (A) a maleated natural oil, comprising a natural oil with maleated functionality; and (B) a functionalized or unfunctionalized moiety selected from the group consisting of hydrophobic moieties, hydrophilic moieties, and combinations thereof, wherein the maleated functionality has been partially reacted with the functionalized or unfunctionalized moiety; and (C) a base, where in the reaction product is of

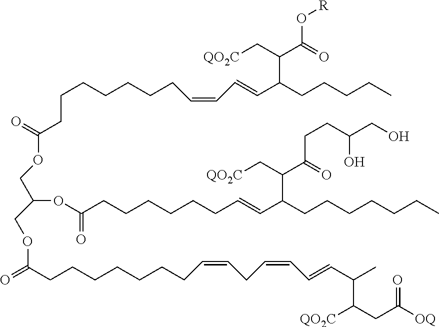

wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and wherein each Q is independently selected from the group consisting of Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

In an another aspect of the present invention provides an composition comprising a reaction product of: (A) a maleated natural oil, comprising a natural oil with maleated functionality; and (B) a functionalized or unfunctionalized moiety selected from the group consisting of hydrophobic moieties, hydrophilic moieties, and combinations thereof, wherein the maleated functionality has been partially reacted with the functionalized or unfunctionalized moiety; and (C) a base, where in the reaction product is a polymer of

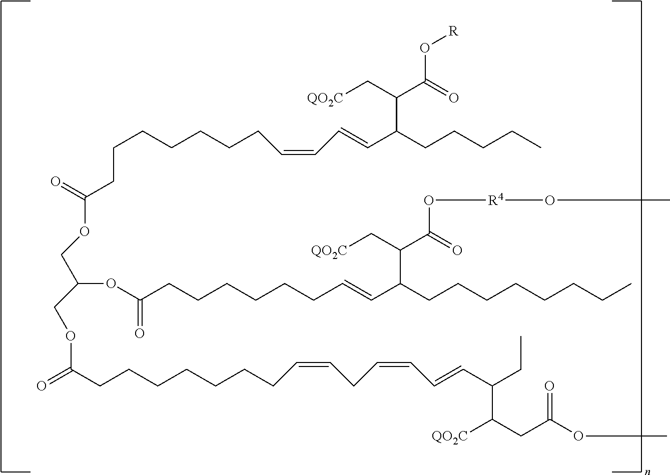

wherein n is greater than 1; wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

In yet another aspect of the invention, the present application provides a composition for topical application to an animal or a plant comprising a safe and effective amount of (A) from about 0.01 to about 20.0 wt. % of the reaction product is a polymer of

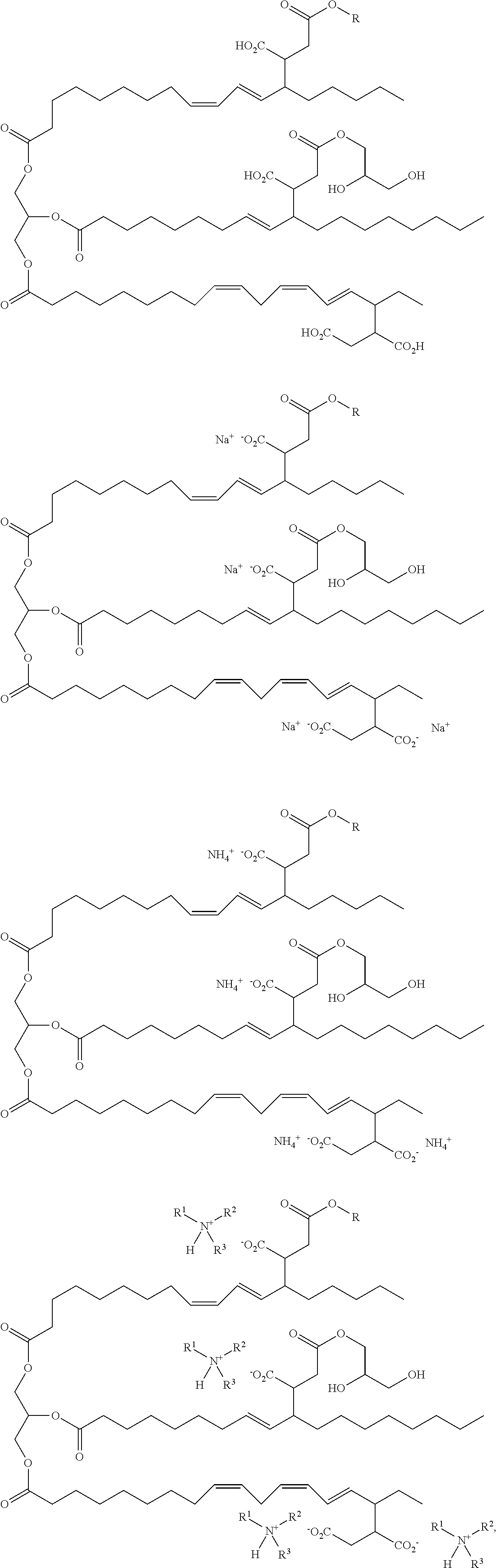

wherein R is ethyl, butyl, hexyl, octyl, 2-ethylhex-1-yl, 2-butyloct-1-yl, 2-hyxyldec-1-yl, 2-octyldodec-1-yl, or mixtures thereof; and wherein R¹, R² and R³ are each, independently, hydrogen, methyl, or an unsubstituted or substituted alkyl group containing 2 to 18 carbon atoms and, optionally, containing heteroatoms; (B) from about 1.0 to about 90.0 wt. % of one or more agriculturally active ingredient; and (C) from about 1.0 to about 99.0 wt. % of one or more additional ingredient.

In yet another aspect of the invention, the present application provides a composition for topical application to an animal or a plant comprising a safe and effective amount of (A) from about 0.01 to about 20.0 wt. % of the reaction product is a polymer of

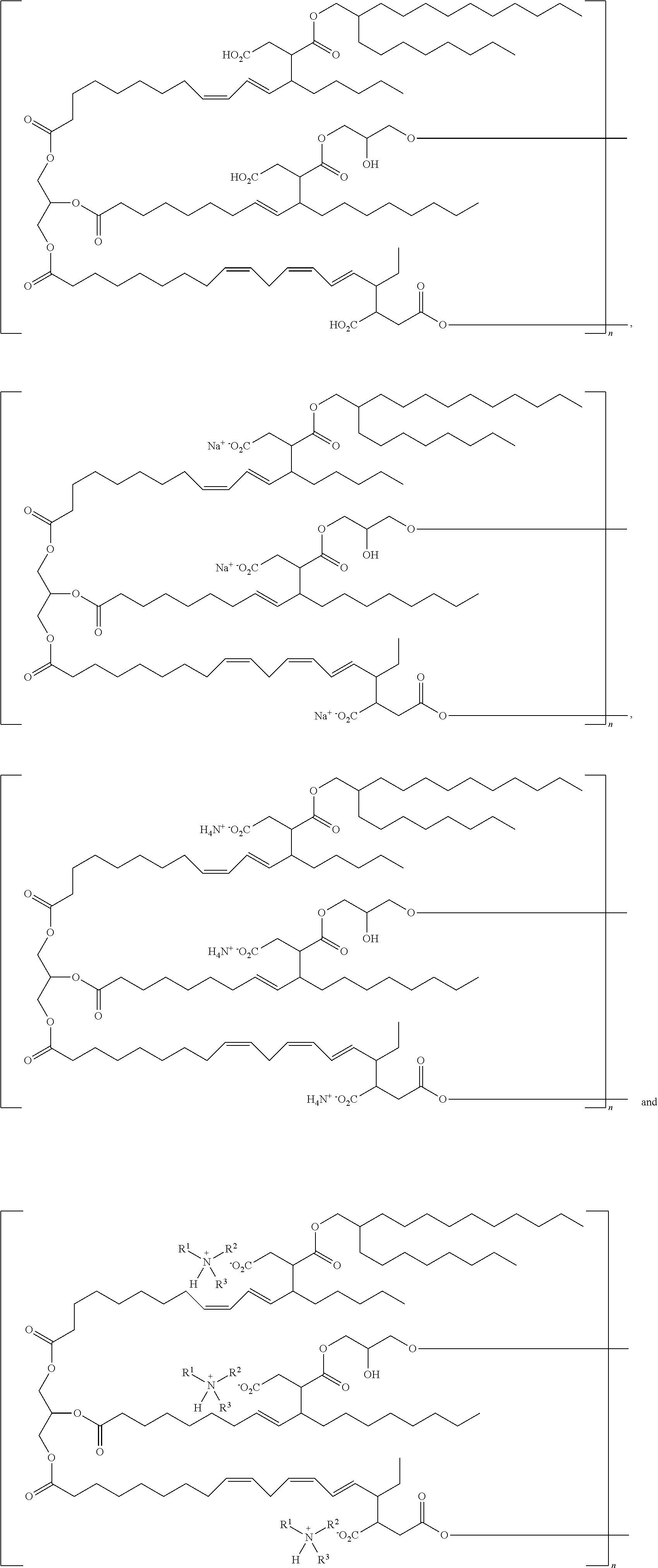

wherein 1<n<30; wherein R is ethyl, butyl, hexyl, octyl, 2-ethylhex-1-yl, 2-butyloct-1-yl, 2-hyxyldec-1-yl, 2-octyldodec-1-yl, or mixtures thereof; wherein R¹, R² and R³ are each, independently, hydrogen, methyl, or an unsubstituted or substituted alkyl group containing 2 to 18 carbon atoms and, optionally, containing heteroatoms; and wherein R⁴ is

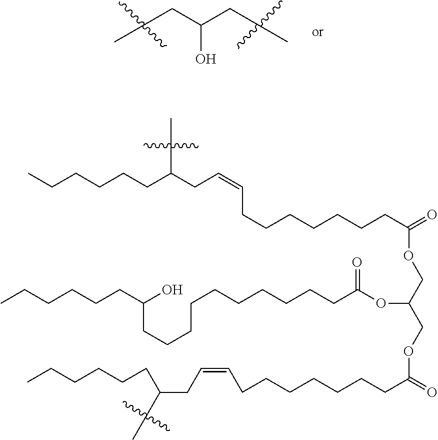 [SMILES: *CC(O)C* ; *C(C/C=C\CCCCCCCC(=O)OCC(COC(=O)CCCCCCC/C=C\CC(*)CCCCCC)OC(=O)CCCCCCCCCCC(O)CCCCCC)CCCCCC]

or a mixture thereof; (B) from about 1.0 to about 90.0 wt. % of one or more agriculturally active ingredient; and (C) from about 1.0 to about 99.0 wt. % of one or more additional ingredient.

In an another aspect of the present invention provides an composition can be contemplated to applications selected from the group consisting of skin care compositions, oral care compositions, hair care compositions, energy compositions, lithium-ion battery compositions, construction compositions, biocidal compositions, preservative compositions, nutraceutical compositions, food compositions, agricultural compositions, coating compositions, ink compositions, cosmetic compositions, homecare compositions, industrial and institutional compositions, metal working compositions, textile compositions, laundry compositions, cleaning compositions, and disinfecting compositions.

In an another aspect of the present invention provides an agricultural composition comprising a reaction product of: (A) a maleated natural oil, comprising a natural oil with maleated functionality; and (B) a functionalized or unfunctionalized moiety selected from the group consisting of hydrophobic moieties, hydrophilic moieties, and combinations thereof, wherein the maleated functionality has been partially reacted with the functionalized or unfunctionalized moiety; and (C) a base, where in the reaction product is of

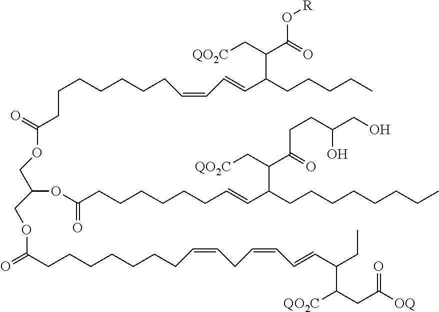

wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

In an another aspect of the present invention provides an agricultural composition comprising a reaction product of: (A) a maleated natural oil, comprising a natural oil with maleated functionality; and (B) a functionalized or unfunctionalized moiety selected from the group consisting of hydrophobic moieties, hydrophilic moieties, and combinations thereof, wherein the maleated functionality has been partially reacted with the functionalized or unfunctionalized moiety; and (C) a base, where in the reaction product is a polymer of

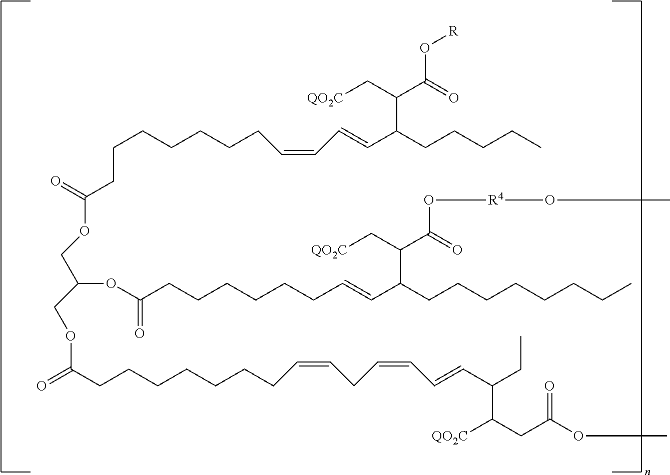

wherein n is greater than 1;
wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

In yet another aspect of the invention, the present application provides an agricultural composition for topical application to an animal or a plant comprising a safe and effective amount of (A) from about 0.01 to about 20.0 wt. % of the reaction product of

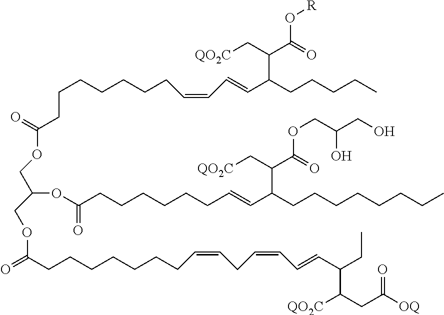

wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and wherein each Q is independently selected from the group consisting of Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium; (B) from about 1.0 to about 90.0 wt. % of one or more agriculturally active ingredient; and (C) from about 1.0 to about 99.0 wt. % of one or more additional ingredient.

In yet another aspect of the invention, the present application provides an agricultural composition for topical application to an animal or a plant comprising a safe and effective amount of (A) from about 0.01 to about 20.0 wt. % of the reaction product is a polymer of

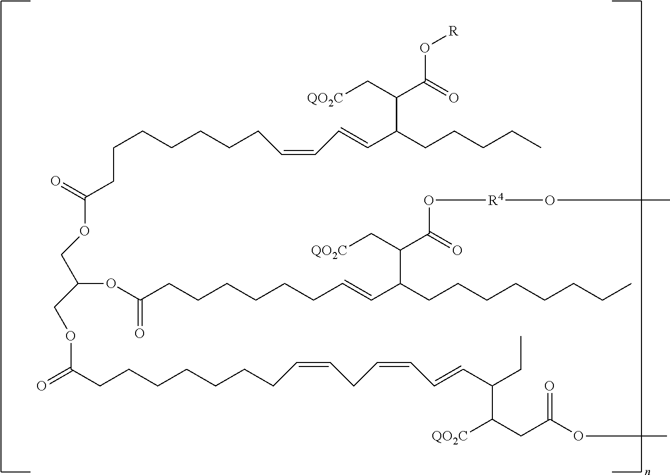

wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

In yet another aspect of the invention, the present application provides an agricultural composition for topical application to an animal or a plant comprising a safe and effective amount of (A) from about 0.01 to about 20.0 wt. % of the reaction product of

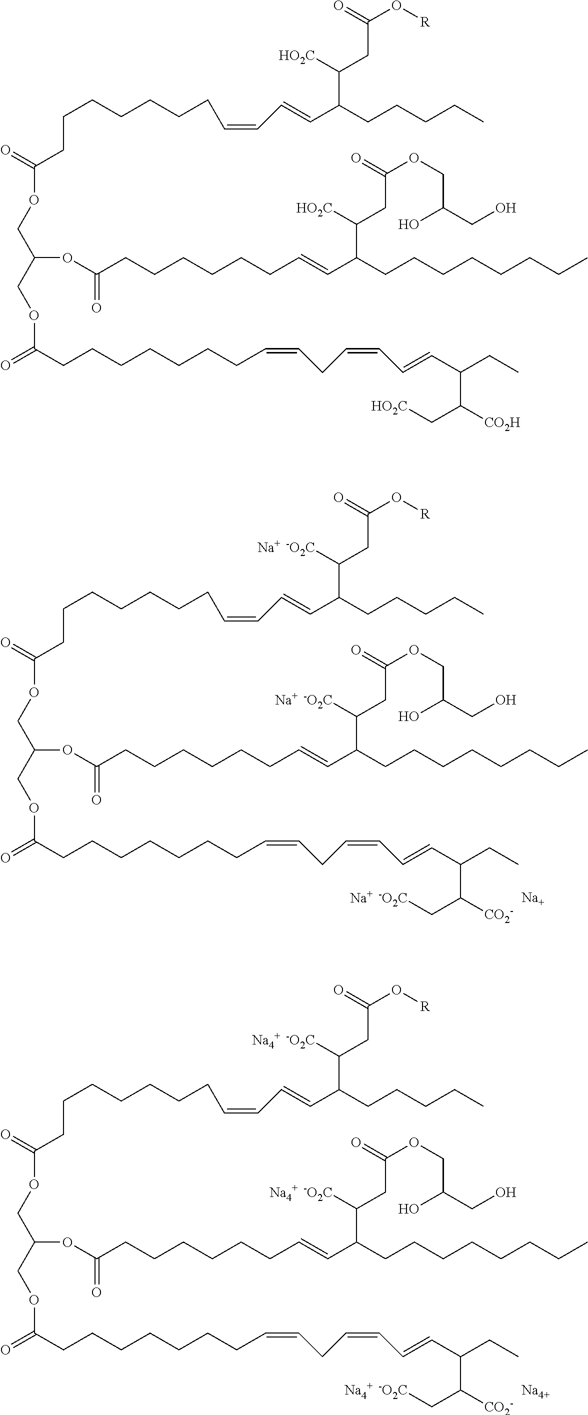

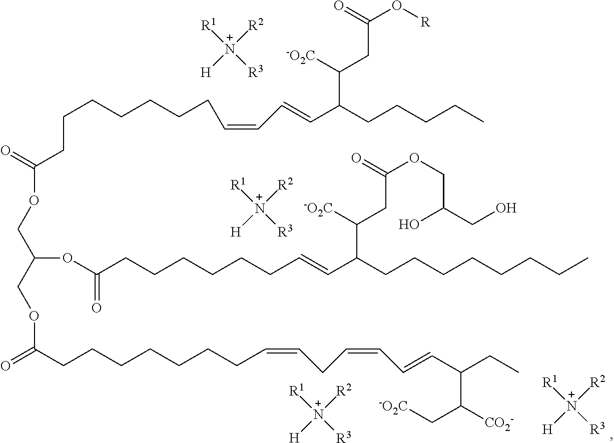

wherein R is ethyl, butyl, hexyl, octyl, 2-ethylhex-1-yl, 2-butyloct-1-yl, 2-hyxyldec-1-yl, 2-octyldodec-1-yl, or mixtures thereof; and wherein R¹, R² and R³ are each, independently, hydrogen, methyl, or an unsubstituted or substituted alkyl group containing 2 to 18 carbon atoms and, optionally, containing heteroatoms; (B) from about 1.0 to about 90.0 wt. % of one or more agriculturally active ingredients; and (C) from about 1.0 to about 99.0 wt. % of one or more additional ingredients.

In yet another aspect of the invention, the present application provides an agricultural composition for topical application to an animal or a plant comprising a safe and effective amount of (A) from about 0.01 to about 20.0 wt. % of the reaction product is a polymer of

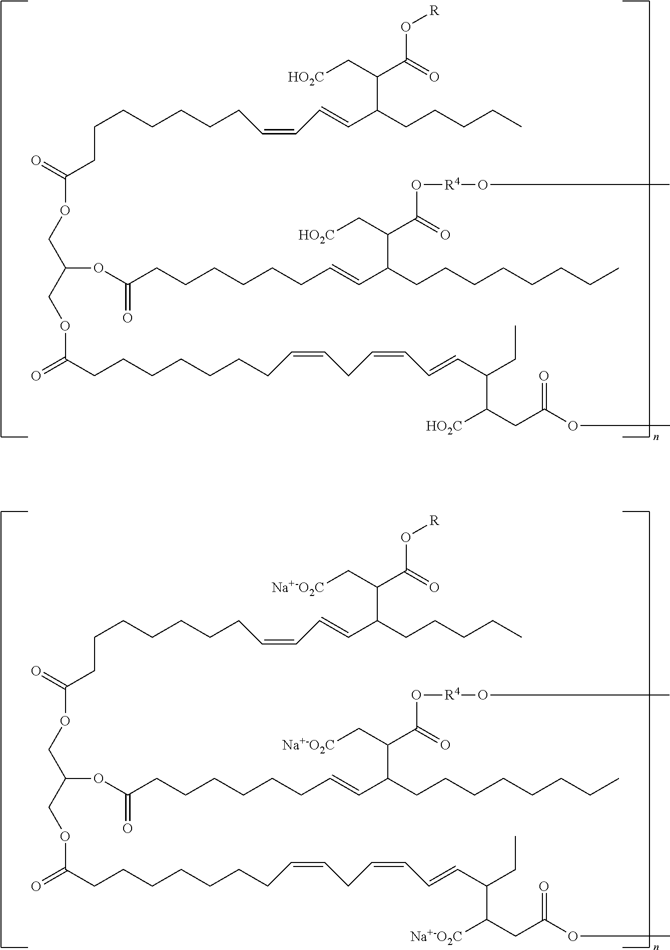

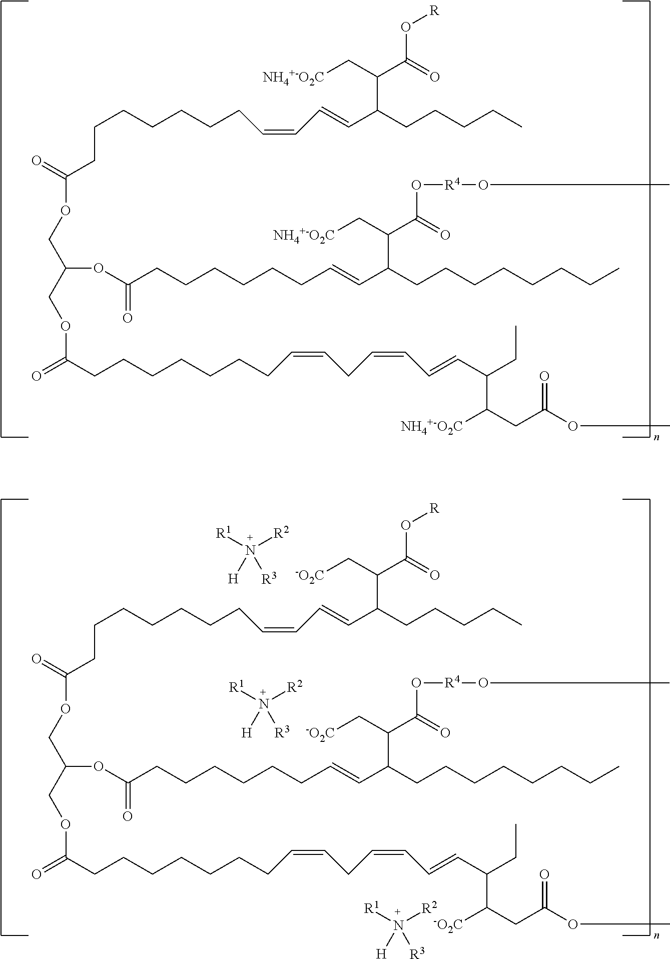

wherein 1<n<30; wherein R is ethyl, butyl, hexyl, octyl, 2-ethylhex-1-yl, 2-butyloct-1-yl, 2-hyxyldec-1-yl, 2-octyldodec-1-yl, or mixtures thereof; wherein R¹, R² and R³ are each, independently, hydrogen, methyl, or an unsubstituted or substituted alkyl group containing 2 to 18 carbon atoms and, optionally, containing heteroatoms; and wherein R⁴ is

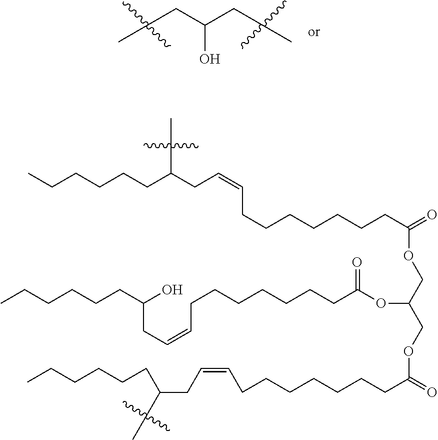 [SMILES: *CC(O)C* ; *C(C/C=C\CCCCCCCC(=O)OCC(COC(=O)CCCCCCC/C=C\CC(*)CCCCCC)OC(=O)CCCCCCC/C=C\CC(O)CCCCCC)CCCCCC]

or a mixture thereof; (B) from about 1.0 to about 90.0 wt. % of one or more agriculturally active ingredients; and (C) from about 1.0 to about 99.0 wt. % of one or more additional ingredients.

In yet another aspect of the invention, the present application provides an agricultural composition selected from the group consisting of an adjuvant composition, a fertilizer composition, a nutrient composition, a plant strengthener composition, a seed coating composition, a soil conditioner composition, a livestock composition, a granular composition, a controlled release composition, a film coating composition, a pesticide composition selected from the group of rodenticide, insecticide, miticide, algicide, molluscicide, acaricide, avicide, fungicide and herbicide compositions, a germicide composition, an antibiotic composition, an antibacterial composition, an antiviral composition, an antifungal composition, an antiprotozoal composition, an anti-parasite composition, a wood preservation composition, an antimicrobial composition or a yield enhancer composition to soil for the plant.

The modified maleated natural oils are useful in a wide variety of agricultural composition and its applications.

### DETAILED DESCRIPTION OF THE INVENTION

The present application provides an agricultural composition comprising modified maleated natural oils which do not exhibit many of the limited properties of maleated natural oils. The modified maleated natural oils are useful in a wide variety of agricultural compositions and its applications.

Natural oils are abundant, inexpensive, and are derived from sustainable sources. Natural oils are useful for synthesizing renewable compounds such as polymers, plastics, and plasticizers, which compounds are useful in a variety of agricultural compositions. A difficultly with utilizing natural oils is that they are blends of triglycerides containing varying degrees of unsaturated groups, which unsaturated groups are relatively unreactive. To make these natural oils reactive, these unsaturated groups are generally chemically modified to make them reactive. For example, these unsaturated groups can be reacted to provide epoxide functional groups or succinic anhydride functional groups. Despite these chemical modifications, the maleated natural oils can still exhibit limited properties such as insolubility or non-dispersibility in water and alcohols. Accordingly, there is a need for further modified maleated natural oils which do not exhibit the limited properties of maleated natural oils.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

The term “branched and unbranched alkyl groups” refers to alkyl groups, which may be straight chained or branched. Branched groups include isopropyl, tert-butyl, and the like.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

The term “polymer” refers to a compound comprising repeating structural units (monomers) connected by covalent chemical bonds. Polymers may be further derivatized, crosslinked, grafted or end-capped. Non-limiting examples of polymers include copolymers, terpolymers, tetrapolymers, quaternary polymers, and homologues. The term “copolymer” refers to a polymer consisting essentially of two or more different types of monomers polymerized to obtain said copolymer.

The term “reaction product” refers to a substance produced from a chemical reaction of one or more reactant substances.

The term “natural oil” refers to compounds comprising triglycerides and may contain varying levels of fatty acids, monoglycerides, diglycerides and triglycerides refer to oil derived from plants or animal sources. Natural oils also include fatty acid glyceryl esters, which are synthesized by reacting glycerol with 1, 2, or 3 molar equivalents of a fatty acid or a mixture of fatty acids. These compounds can be mono, di or triglycerides of a single fatty acid or a mixture of fatty acids.

The term “maleated natural oil”, as used herein, refers to natural oil containing at least one or more maleated functionality. Accordingly, the term “maleation” or “maleated” as used hereafter should be understood to mean “functionalization” insofar as the use of functionalizing reagents other than maleic anhydride are contemplated for use in the process of the invention.

As used herein, the term “moiety” or “moieties” refers to a part or a functional group(s) of a molecule.

The term, “maleated functionality” refers to moieties formed by attaching maleic anhydride to unsaturated fatty acyl chains, found in natural oils, by ene reaction. “Maleated functionality” includes but is not limited to the cyclic anhydride form (I), the diacid form (II), the disodium dicarboxylate form (III), other dicarboxylate salt forms, and the half ester form (IV). Since the carbon-carbon double bond of maleic anhydride becomes a saturated carbon-carbon single bond during the ene reaction, the “maleated functionality” pictured in I, II, III and IV might also be called by one of ordinary skill in the art succinic anhydride, succinic acid, succinate salt or succinate half ester functionality.

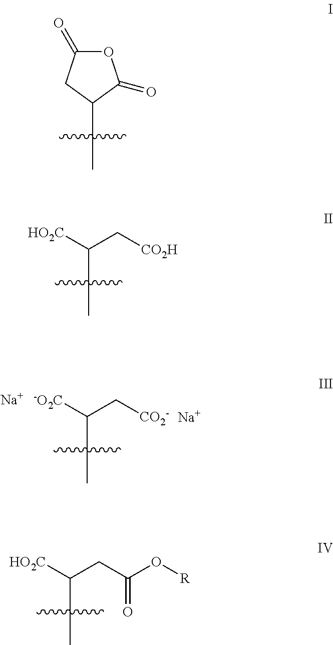

The term “base”, as used herein, refers to any substance which can alter the pH of a solution from a neutral pH of 7.0 to a basic pH (i.e., 7.1 to 14). Typically, a base is a substance of a large class of compounds with one or more of the following properties: bitter taste, slippery feeling in solution, ability to turn litmus blue and to cause other indicators to take on characteristic colors, ability to react with (neutralize) acids to form salts includes both organic base or an inorganic base and mixtures thereof.

The term “organic base”, as used herein, includes ammonia, primary amines, secondary amines, pyridine, imidazole, benzimidazole, histidine, guanidine, and mixtures thereof.

The term “inorganic base”, as used herein, includes oxides of alkali metals and alkaline earth metals, hydroxides of alkali metals and alkaline earth metals, carbonates of alkali metals and alkaline earth metals, bicarbonates of alkali metals and alkaline earth metals, oxides of transition metals, hydroxides of transition metals, carbonates of transition metals, bicarbonates of transition metals, and combinations thereof.

The term “alkali metal base” includes oxides, hydroxides, carbonates, or bicarbonates of sodium (Na), potassium (K), rubidium (Rb), cesium (Cs), and francium (Fr).

The term “alkali-earth metal base” includes oxides, hydroxides, carbonates, or bicarbonates of beryllium (Be), magnesium (Mg), calcium (Ca), strontium (Sr), barium (Ba), and radium (Ra).

The term “transition metal base” includes oxides, hydroxides, carbonates, or bicarbonates of scandium (Sc), titanium (Ti), vanadium (V), chromium (Cr), manganese (Mn), iron (Fe), cobalt (Co), nickel (Ni), copper (Cu), zinc (Zn), yttrium (Y), zirconium (Zr), niobium (Nb), molybdenum (Mo), technetium (Tc), ruthenium (Ru), rhodium (Rh), palladium (Pd), silver (Ag), cadmium (Cd), hafnium (Hf), tantalum (Ta), tungsten (W), rhenium (Re), osmium (Os), iridium (Ir), platinum (Pt), gold (Au), mercury (Hg), rutherfordium (Rf), dubnium (Db), seaborgium (Sg), bohrium (Bh), hassium (Hs), meitnerium (Mt), darmstadtium (Ds) and roentgenium (Rg).

The term “agriculturally active ingredient”, as used herein refers to a chemical used in agriculture, horticulture, and pest management for protection of crops, plants, structures, animals and humans against unwanted organisms such as fungal and bacterial plant pathogens, weeds, insects, mites, algae, nematodes, and the like. Specifically an active ingredient selected from the group consisting of fertilizers, herbicides, ovicides, fungicides, pesticides selected from the group of rodenticides, miticides, algicides, molluscicides, acaricides, avicides, insecticides: germicides, antibiotics, antibacterial, antivirals, antifungals, antiprotozoals, anti-parasites and antimicrobials compounds wherein such agent is applicable to a plant, a seed, a soil or other growth medium, to improve for agricultural production purposes the physical, chemical, or biological characteristics thereof in order to improve crop production, plant growth, product quality, product durability, or yield prior to harvest.

The term “additional ingredient”, as used herein refers to an adjuvant or an inert ingredient substance which can increase the biological activity of the active ingredient but are themselves not significantly biologically active. An agricultural ingredient assists with the effectiveness of the active ingredient such as, for example, by improving the delivery and uptake of an agriculturally active ingredient into a target plant or animal leading to improved biological control.

The agricultural composition comprising an ingredient refers to an adjuvant is selected from the group of acidifying agents, buffering agents, anti-foam agents, defoaming agents, anti-transpirants, dyes and brighteners, compatibility agents, crop oil concentrates, oil surfactants, deposition agents, drift reduction agents, foam markers, feeding stimulants, herbicide safeners, spreaders, extenders, adhesive agents, suspension agents, gelling agents, synergists, wetting agents, emulsifiers, dispersing agents, penetrants, tank and equipment cleaners, neutralizers, water absorbents, water softeners and mixtures thereof.

The agricultural composition further comprises the ingredient(s) can be selected from the group consisting of solvents, liquid carriers, solid carriers or fillers, surfactants, solubilizers, penetration enhancers, protective colloids, thickeners, humectants, repellents, attractants, compatibilizers, bactericides, anti-freezing agents, crystallization inhibitors, colorants, tackifiers, binders, preservatives, pH adjuster, clarifiers, stabilizers, UV stabilizers, and mixtures thereof.

The term “functionalized” with reference to any moiety refers to the presence of one or more functional groups in the moiety. Various functional groups may be introduced in a moiety by way of one or more functionalization reactions known to a person having ordinary skill in the art. Non-limiting examples of functionalization reactions include: alkylation, epoxidation, sulfonation, hydrolysis, amidation, esterification, hydroxylation, dihydroxylation, amination, ammonolysis, acylation, nitration, oxidation, dehydration, elimination, hydration, dehydrogenation, hydrogenation, acetalization, halogenation, dehydrohalogenation, Michael addition, aldol condensation, Canizzaro reaction, Mannich reaction, Clasien condensation, Suzuki coupling, and the like. In one non-limiting embodiment, the term “functionalized” with reference to any moiety refers to the presence of one more functional group selected from the group consisting of alkyl, alkenyl, hydroxyl, carboxyl, halogen, alkoxy, amino, imino, and combinations thereof, in the moiety.

As used herein, the term “hydrophilic” means that the compound has an affinity for water, whereas “hydrophobic” means not having an affinity for water.

The terms are relative terms, where a hydrophilic moiety it has a higher affinity for water than a hydrophobic moiety, but the hydrophilic moiety may or may not be completely water soluble. Likewise, hydrophobic moieties have less of an affinity for water than hydrophilic moieties, but the hydrophobic moieties may not necessarily be water-repellant. While hydrophilic moieties have an affinity for water and other polar solvents, hydrophobic moieties tend to have an affinity for oils, fats, and other non-polar solvents.

The term “unreacted maleated functionality” refers to an unreacted maleic anhydride functionality in the reaction product of this invention, wherein the anhydride functionality is completely unreacted and intact. Since the carbon-carbon double bond of maleic anhydride becomes saturated during its attachment to unsaturated fatty acid chains during the “ene” or “maleation” reaction, the “unreacted maleated functionality” could also be considered intact succinyl anhydride moiety or moieties.

The term “hydrocarbyl” includes straight-chain and branched-chain alkyl, alkenyl, alkynyl, cycloalkyl, cycloalkenyl, aryl groups, and combinations thereof with optional heteroatom(s). A hydrocarbyl group may be mono-, di- or polyvalent and have carbon chains contain at least 2 carbon atoms and preferably 2 to 100 carbon atoms.

The term “alkyl” refers to a functionalized or unfunctionalized, monovalent, straight-chain, branched-chain, or cyclic C1-C60 hydrocarbyl group optionally having one or more heteroatoms. In one non-limiting embodiment, an alkyl is a C1-C45 hydrocarbyl group. In another non-limiting embodiment, an alkyl is a C1-C30 hydrocarbyl group. Non-limiting examples of alkyl include methyl, ethyl, n-propyl, isopropyl, n-butyl, isobutyl, tert-butyl, n-pentyl, isopentyl, n-hexyl, n-heptyl, n-octyl, 2-ethylhexyl, tert-octyl, iso-norbornyl, n-dodecyl, tert-dodecyl, n-tetradecyl, n-hexadecyl, n-octadecyl, n-eicosyl, cyclobutyl, cyclopentyl, cyclohexyl, and the like. The definition of “alkyl” also includes groups obtained by combinations of straight-chain, branched-chain and/or cyclic structures.

The term “aryl” refers to a functionalized or unfunctionalized, monovalent, aromatic hydrocarbyl group optionally having one or more heteroatoms. The definition of aryl includes carbocyclic and heterocyclic aromatic groups. Non-limiting examples of aryl groups include phenyl, naphthyl, indenyl, indanyl, azulenyl, fluorenyl, anthracenyl, furyl, thienyl, pyridyl, pyrrolyl, oxazolyl, thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, cinnolinyl, phthalazinyl, quinazolinyl, quinoxalinyl, 1,8-naphthridinyl, pteridinyl, carbazolyl, acridinyl, phenazinyl, phenothiazinyl, phenoxyazinyl, pyrazolo[1,5-c]triazinyl, and the like.

The term “aralkyl” refers to an alkyl group comprising one or more aryl substituent(s) wherein “aryl” and “alkyl” are as defined above. Non-limiting examples of aralkyl groups include benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like.

The term “alkylene” refers to a functionalized or unfunctionalized, divalent, straight-chain, branched-chain, or cyclic C1-C40 hydrocarbyl group optionally having one or more heteroatoms. In one non-limiting embodiment, an alkylene is a C1-C30 group. In another non-limiting embodiment, an alkylene is a C1-C20 group. Non-limiting examples of alkylene groups include:

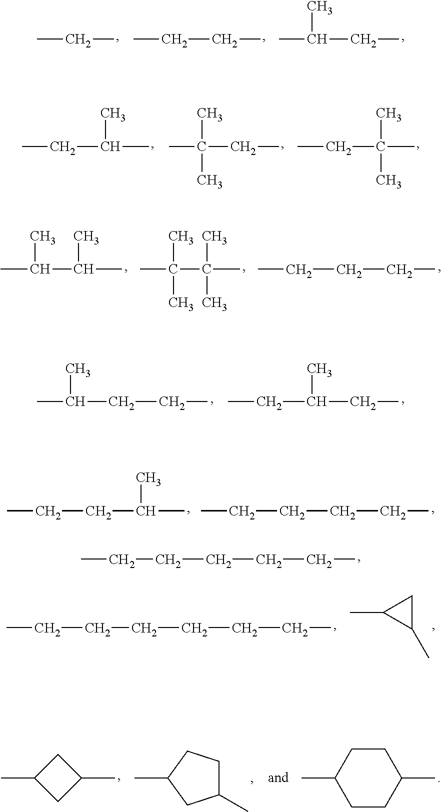

The term “arylene” refers to a functionalized or unfunctionalized, divalent, aromatic hydrocarbyl group optionally having one or more heteroatoms. The definition of arylene includes carbocyclic and heterocyclic groups. Non-limiting examples of arylene groups include phenylene, naphthylene, pyridinylene, and the like.

The term “heteroatom” refers to oxygen, nitrogen, sulfur, silicon, phosphorous, or halogen. The heteroatom(s) may be present as a part of one or more heteroatom-containing functional groups. Non-limiting examples of heteroatom-containing functional groups include ether, hydroxy, epoxy, carbonyl, carboxamide, carboxylic ester, carboxylic acid, imine, imide, amine, sulfonic, sulfonamide, phosphonic, and silane groups. The heteroatom(s) may also be present as a part of a ring such as in heteroaryl and heteroarylene groups.

The structures set out below may refer to the first, second, or third structure, or combinations thereof. The succinic anhydride group may be present in the top, middle, or lower chain.

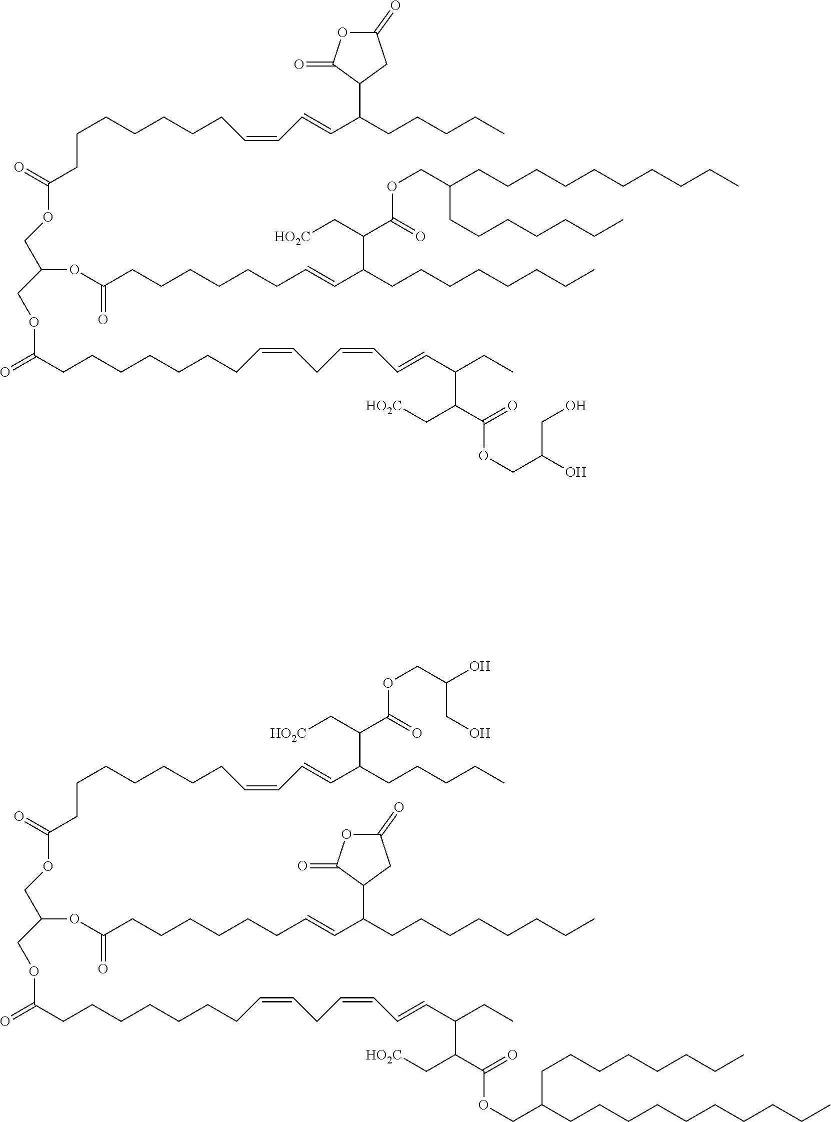 [SMILES: CCCCCCCCCCC(CCCCCCC)COC(=O)C(CC(=O)O)C(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C(CC(=O)O)C(=O)OCC(O)CO)CCCCCCCC ; CCCCCCCCCCC(CCCCCCCC)COC(=O)C(CC(=O)O)C(/C=C/C=C\C/C=C\CCCCCCCC(=O)OCC(COC(=O)CCCCCCC/C=C\C=C\C(CCCCC)C(CC(=O)O)C(=O)OCC(O)CO)OC(=O)CCCCCC/C=C/C(CCCCCCCC)C1CC(=O)OC1=O)CC]

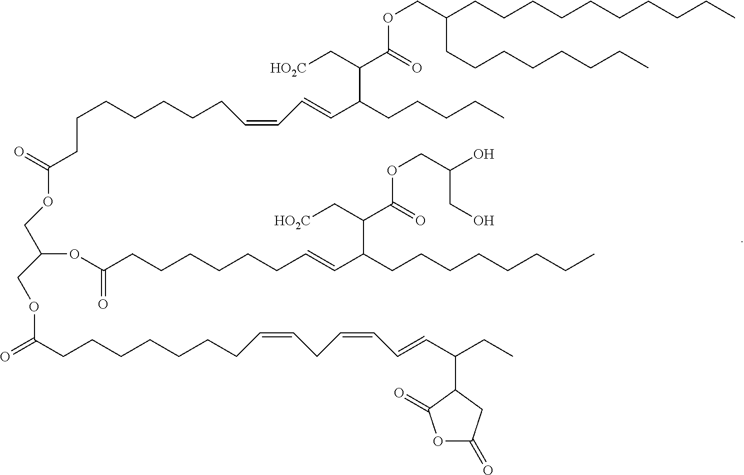 [SMILES: CCCCCCCCCCC(CCCCCCCC)COC(=O)C(CC(=O)O)C(/C=C/C=C\CCCCCCCC(=O)OCC(COC(=O)CCCCCCC/C=C\C/C=C\C=C\C(CC)C1CC(=O)OC1=O)OC(=O)CCCCCC/C=C/C(CCCCCCCC)C(CC(=O)O)C(=O)OCC(O)CO)CCCCC]

In one non-limiting embodiment, the hydrophobic moiety and the hydrophilic moiety is a hydrocarbyl alcohol, a hydrocarbyl amine, a silicon-based compound, or a combination thereof.

Hydrocarbyl alcohols are classified as primary, secondary and tertiary alcohols, based on the number of carbon atoms connected to the carbon atom that bears the hydroxyl group. Each classification of alcohol may have a general formula. For example,
the general formula for primary alcohols is

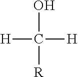

the general formula for secondary alcohols is

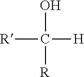

and
the general formula for tertiary alcohols is

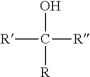

wherein R, R′ and R″ stand for different alkyl, alkylene, aryl, aralkyl, and arylene, groups.

The present invention provides agricultural composition comprising a reaction product of a maleated natural oil; and a functionalized or unfunctionalized moiety selected from the group consisting of hydrophobic moieties, hydrophilic moieties, and combinations thereof. In one non-limiting embodiment, the reaction product comprises an unreacted maleated functionality or a maleated functionality functionalized with a hydrophobic moiety, a hydrophilic moiety, or combinations thereof.

Preferably, the hydrophobic moiety is a moiety selected from the group consisting of unsubstituted or substituted alkyl, cycloalkyl, alkenyl, and aryl alcohols and amines, wherein the alcohols and amines contain from about 6 to about 36 carbon atoms and may contain heteroatoms; silicon-based compounds; and combinations thereof.

Preferably, the hydrophilic moiety is a moiety selected from the group consisting of selected from those of the group consisting of unsubstituted or substituted alkyl, cycloalkyl, alkenyl, and aryl alcohols and amines, wherein the alcohols and amines contain from about 1 to about 5 carbon atoms and may contain heteroatoms; unsubstituted or substituted polyols, wherein the unsubstituted or substituted polyols contain from about 2 to about 36 carbon atoms and may contain heteroatoms; silanes; and combinations thereof.

Preferably, the silane is functionalized with an alcohol or an amine, and combinations thereof. Preferably, the hydrophobic alcohol is selected from the group consisting of hexanol, heptanol, nonanol, decanol, dodecanol, phenol, ethylbenzyl alcohol, 2-ethyl-1-hexanol, 1-octanol, 2-octanol, 2-butyl-1-octanol, 2-octyl-1-dodecyl alcohol, 1-tetradecanol, 2-tetradecanol, 1-hexadecanol, 2-hexadecanol, behenyl alcohol, 3,7-dimethyl-1-octanol, 2-propyl-1-pentanol, 4-methyl-1-pentanol, and mixtures thereof. Preferably, the hydrophilic alcohol is selected from the group consisting of methanol, ethanol, propanol, isopropanol, butanol, methoxypolyethylene glycol, and mixtures thereof. Preferably, the hydrophilic polyol is selected from the group consisting of ethylene glycol, propylene glycol, diethylene glycol, dipropylene glycol, dibutylene glycol, polyethylene glycol, polypropylene glycol, hexylene glycol, sorbitol, neopentylglycol, eythritol, mannitol, xylitol, threitol, pentaerythritol, beta-cyclodextrin, ribose, 2-deoxygalactose, and mixtures thereof.

Preferably, the hydrophobic amine is selected from the group consisting of benzylamine, cyclohexylamine, hexylamine, methylhexylamine, phenethylamine, octylamine, oleylamine, decylamine, dodecylamine, octadecylamine, undecylamine, pentadecylamine, 2-methylbutylamine, and mixtures thereof. Preferably, the hydrophilic amine is selected from the group consisting of diethanolamine, serinol hydrochloride, 2-amino-2-ethyl-1,3-propanediol, dimethylamine, and mixtures thereof.

Preferably, the hydrophobic polyol is selected from the group consisting of 1,6-hexanediol, 1,7-heptanediol, 1,8-octanediol, 1,9-nonanediol, 1,10-decanediol, 1,12-dodecanediol, poly(tetramethylene ether) glycol, poly(tetramethylene carbonate), poly(hexamethylene carbonate) and castor oil.

Preferably, the hydrophobic silicon-based compound is selected from the group consisting of aminopropylmethylsiloxane-dimethylsiloxane, N-ethylaminoisobutyl terminated polydimethylsiloxane, poly(1,1-dimethylsilazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine]terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof. Preferably, the hydrophilic silane is selected from the group consisting of 3-aminopropylsilanetriol, N-(2-aminoethyl)-3-aminopropylsilanetriol, and mixtures thereof.

In one non-limiting embodiment, the hydrophobic moiety is that of a hydrocarbyl alcohol that contains from about 6 to about 36 carbon atoms and that is linear, branched, saturated, unsaturated, aliphatic, aromatic, monofunctional or multifunctional.

In one non-limiting embodiment, the hydrophilic polyol is selected from the group consisting of ethylene glycol, propylene glycol, diethylene glycol, dipropylene glycol, dibutylene glycol, polyethylene glycol, polypropylene glycol, hexylene glycol, sorbitol, neopentylglycol, eythritol, mannitol, xylitol, threitol, pentaerythritol, beta-cyclodextrin, ribose, 2-deoxygalactose and mixtures thereof.

In one non-limiting embodiment, the amines selected from the group consisting of primary, secondary, and tertiary amines and combinations thereof.

Hydrocarbyl amines are classified as primary, secondary, or tertiary, based on the number of carbon atoms attached to the amine nitrogen atom. Each class of amine may have a general formula. For example,
the general formula for primary amine is

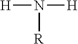

and
the general formula for secondary amine is

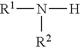

the general formula for tertiary amine is

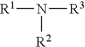

wherein R and R′ stand for the same or different alkyl, alkylene, aryl, aralkyl, or arylene groups.

In one non-limiting embodiment, the hydrophobic moiety is that of a hydrocarbyl amine that contains about 6 to about 36 carbon atoms and that is linear, branched, saturated, unsaturated, aliphatic, aromatic, monofunctional or multifunctional.

In one non-limiting embodiment, the silicon-based compound is a compound having the structure

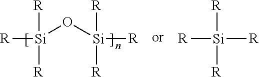

wherein R stands for different alkyl, alkylene, aryl, aralkyl, arylene, hetero groups functionalized with at least one or more alcohol, amine or a combination thereof and n has the value of 1 to 10.

In one non-limiting embodiment, the silicon-based compound is a siloxane, or a silane functionalized with an alcohol, an amine or a combination thereof.

In one non-limiting embodiment, the silicon-based compound is a linear, branched, saturated, unsaturated, aliphatic, aromatic, monofunctional or multifunctional compound.

In one non-limiting embodiment, the hydrocarbyl alcohol is a hydrophobic alcohol selected from the group consisting of hexanol, heptanol, nonanol, decanol, dodecanol, phenol, ethylbenzyl alcohol, 2-ethyl-1-hexanol, 1-octanol, 2-octanol, 2-butyl-1-octanol, 2-octyl-1-dodecyl alcohol, 1-tetradecanol, 2-tetradecanol, 1-hexadecanol, 2-hexadecanol, behenyl alcohol, 3,7-dimethyl-1-octanol, 2-propyl-1-pentanol, 4-methyl-1-pentanol and mixtures thereof.

In one non-limiting embodiment, the hydrocarbyl alcohol is a hydrophilic alcohol selected from the group consisting of methanol, ethanol, propanol, isopropanol, butanol, methoxypolyethylene glycol, and mixtures thereof. Preferably, the hydrophilic polyol is selected from the group consisting of ethylene glycol, propylene glycol, diethylene glycol, dipropylene glycol, dibutylene glycol, polyethylene glycol, polypropylene glycol, hexylene glycol, glycerol, sorbitol, octanol, methyl ethyl pentanol, trimethyl pentanol, ethyl hexanol, methyl heptanol, nonanol, methyl octanol, ethyl heptanol, methyl ethyl hexanol, cyclohexanol, dimethyl heptanol, decanol, methyl nonanol, ethyl octanol, trimethyl heptanol, undecanol, methyl decanol, ethyl nonanol, dodecanol, tetradecanol, hexadecanol, octadecanol, benzyl alcohol, phenoxyethanol, neopentylglycol, trimethylol propane, N-methyldiethanolamine, erythritol, mannitol, xylitol, pentaerythritol, threitol, pentaerythritol, beta-cyclodextrin, ribose, 2-deoxygalactose and mixtures thereof.

In one non-limiting embodiment, the hydrocarbyl amine is a hydrophobic amine selected from the group consisting of benzylamine, cyclohexylamine, hexylamine, methylhexylamine, phenethylamine, octylamine, oleylamine, decylamine, dodecylamine, octadecylamine, undecylamine, pentadecylamine, 2-methylbutylamine, and mixtures thereof.

In one non-limiting embodiment, the hydrocarbyl amine is a hydrophilic amine selected from the group consisting of 2-methylpentane-1,5-diamine, diethanolamine, serinol hydrochloride, 2-amino-2-ethyl-1, 3-propanediol, dimethylamine, and mixtures thereof.

In one non-limiting embodiment, the silicon-based compound is a hydrophobic compound selected from the group consisting of aminopropylmethylsiloxane-dimethylsiloxane, N-ethylaminoisobutyl terminated polydimethylsiloxane, poly(1,1-dimethylsilazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine]terminated polydimethylsiloxane, silanol terminated polydimethylsiloxane, silanol terminated polydiphenylsiloxane, dodecylmethylsiloxane-hydroxypolyalkyleneoxypropyl methylsiloxane and mixtures thereof.

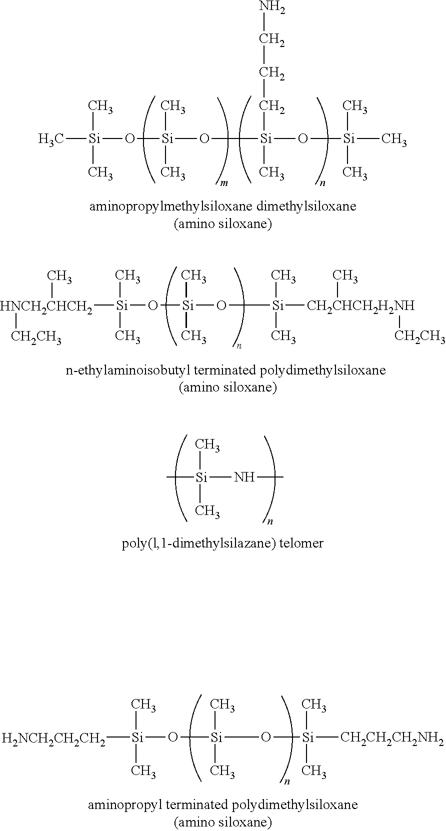

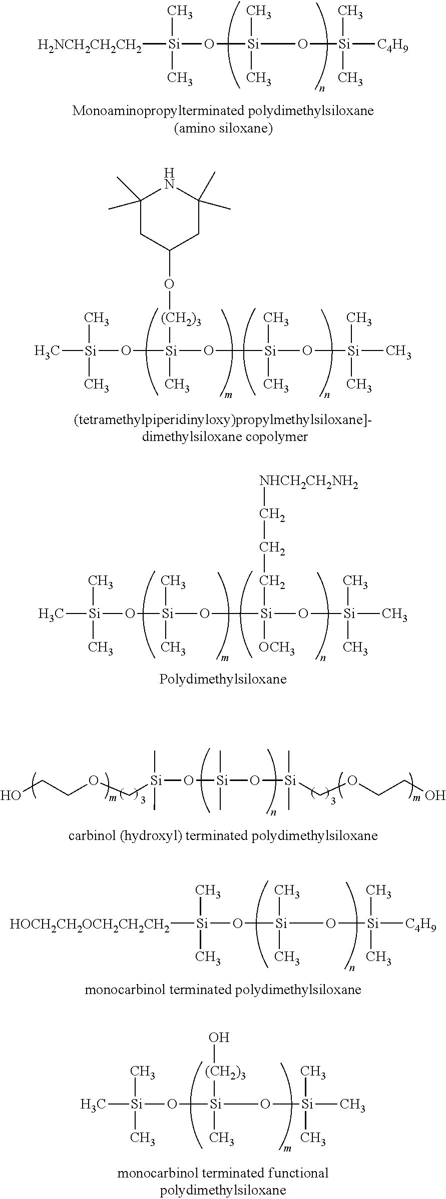

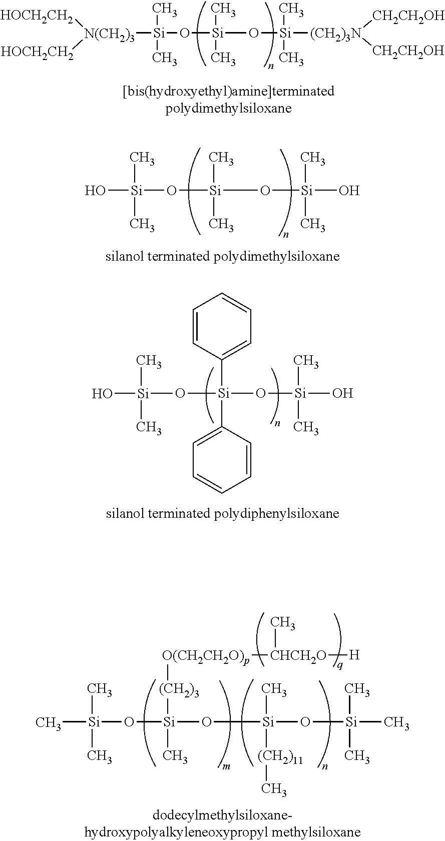

In one non-limiting embodiment, the silicon-based compound is hydrophilic compound selected from the group consisting of 3-aminopropylsilanetriol, N-(2-aminoethyl)-3-aminopropylsilanetriol and mixtures thereof.

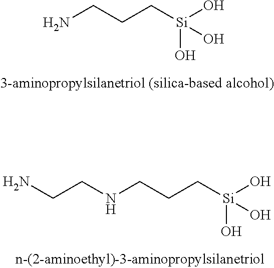 [SMILES: NCCC[Si](O)(O)O ; NCCNCCC[Si](O)(O)O]

Preferably, the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, maleated chickpea oils, maleated clary sage oils and mixtures thereof. More preferably, the maleated natural oil is a maleated soybean oil.

The reactions according to the application may be readily synthesized by procedures known by those skilled in the art, non-limiting examples of which include free radical solution polymerization, dispersion polymerization, emulsion polymerization, ionic chain polymerization, living polymerization, bulk polymerization, suspension polymerization or precipitation polymerization. Particularly, the polymerization is carried out by any one of the methods disclosed in “Principles of Polymerization” 4th edition, 2004, Wiley by George Odian and is referred and disclosed herein in its entirety and described in “Decomposition Rate of Organic Free Radical Polymerization” by K. W. Dixon (section II in Polymer Handbook, volume 1, 4th edition, Wiley-Interscience, 1999), which is herein incorporated in its entirety by reference.

The maleation reaction in natural oil can occur under heating in three different ways. The first one is known as “Ene” reaction (pericyclic reaction between an allylic moiety with an allylic hydrogen and an enophile), producing a triglyceride structure with anhydride moieties (succinic anhydride) and causing a double bond on a fatty acid to migrate. The second one is a radical addition, which consumes a double bond in the fatty acid, incorporating the succinic anhydride into the natural oil structure. The final reaction is also a radical addition that incorporates the maleic anhydride into the natural oil structure without consuming C═C bonds (fatty acid chain and maleic anhydride); this reaction occurs due to deprotonation of a hydrogen between two alkene groups. Subsequently, the maleated natural oil can be reacted with the hydrophobic moiety or the hydrophilic moiety of a hydrocarbyl alcohol, a hydrocarbyl amine, or a silicon-based compound.

The reaction of the non-limiting example above, for illustration purposes only, may be performed at elevated temperatures, such as a temperature between about 150° C. and about 300° C., alternatively between about 170° C. and about 230° C., or alternatively between about 200° C. and about 220° C. The reaction time may be between about 0.5 hours and about 10 hours. In one embodiment, the reaction time is between about 1 hour and about 5 hours and, in another embodiment, between about 2 hours and 4 hours And, in another embodiment, between about 6 hours and 10 hours.

During maleation, the mole ratio of maleic anhydride to natural oil in some embodiments is 1:1. In other embodiments, the mole ratio of maleic anhydride to natural oil is from 1:1 to 2:1; in other embodiments, from 1:1 to 2.8:1; and, in still other embodiments, from 1:1 to 3.2:1.

In some embodiments of the reaction of a maleated natural oil and a hydrophobic moiety, a hydrophilic moiety or a combination thereof, the mole ratio of the maleated natural oil, bearing, on average, about 2 or more equivalents of maleated functionality, and the hydrophobic moiety, hydrophilic moiety or combination thereof is about 1:1, thus forming a reaction product comprising at least about one unreacted maleated functionality.

In one non-limiting embodiment, the invention provides an agricultural composition comprising: (A) a reaction product of a maleated natural oil, comprising a natural oil with maleated functionality and a base; and further comprises (B) one or more agriculturally active ingredient; and (C) one or more additional ingredient.

In one non-limiting embodiment, the invention provides an agriculture composition comprising (A) from about 0.01 to about 20.0 wt. % of a reaction product of (a) a maleated natural oil, comprising a natural oil with maleated functionality; and (b) a base; and (B) from about 0.1 to about 90.0 wt. % of one or more agriculturally active ingredient; and (C) from about 1 to about 99.0 wt. % of one or more additional ingredient.

In one non-limiting embodiment, the invention provides an agricultural composition comprising a reaction product of (A) from about 0.01 to about 20.0 wt. % of the reaction product comprises (a) of a maleated natural oil, comprising a natural oil with maleated functionality; (b) a base selected from the group consisting of sodium hydroxide, potassium hydroxide, calcium hydroxide and mixtures thereof, wherein the maleated functionality has been partially reacted with the base; (B) from about 1.0 to about 90.0 wt. % of one or more agriculturally active ingredient; and (C) from about 1.0 to about 99.0% of one or more additional ingredient.

In one non-limiting embodiment, the invention provides an agricultural composition comprising a reaction product of (A) the reaction product comprises one or more structure selected from the group of structures, comprising maleated functionality fully or partially reacted with a base selected from the group consisting of sodium hydroxide, calcium hydroxide and mixtures thereof, consisting of the following structures:

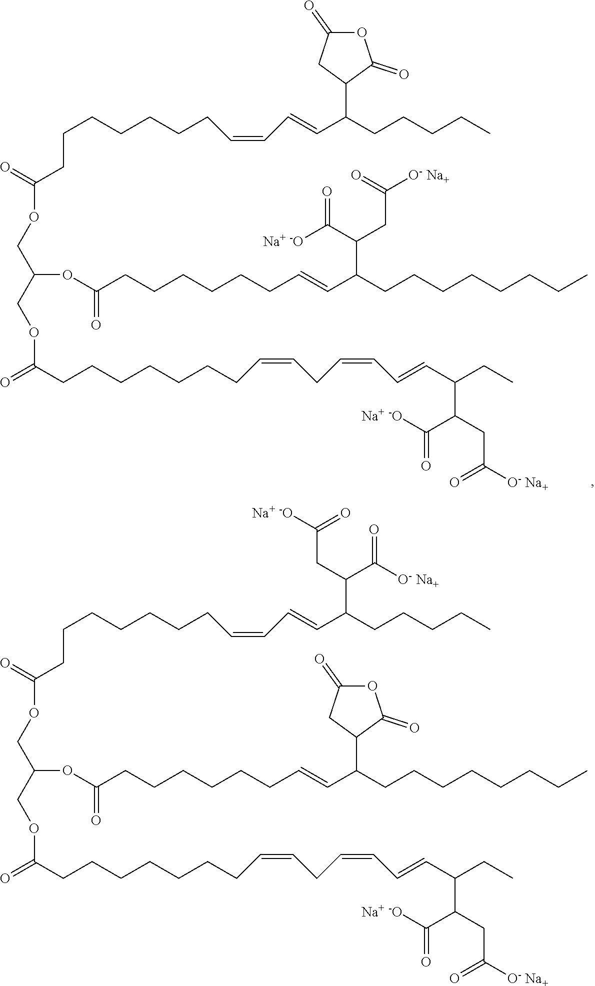

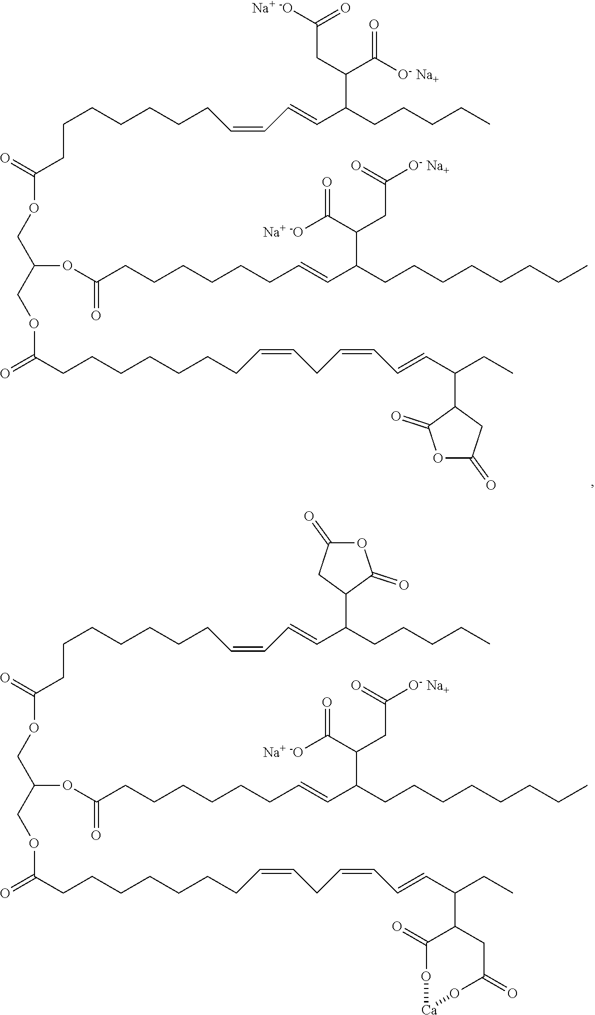

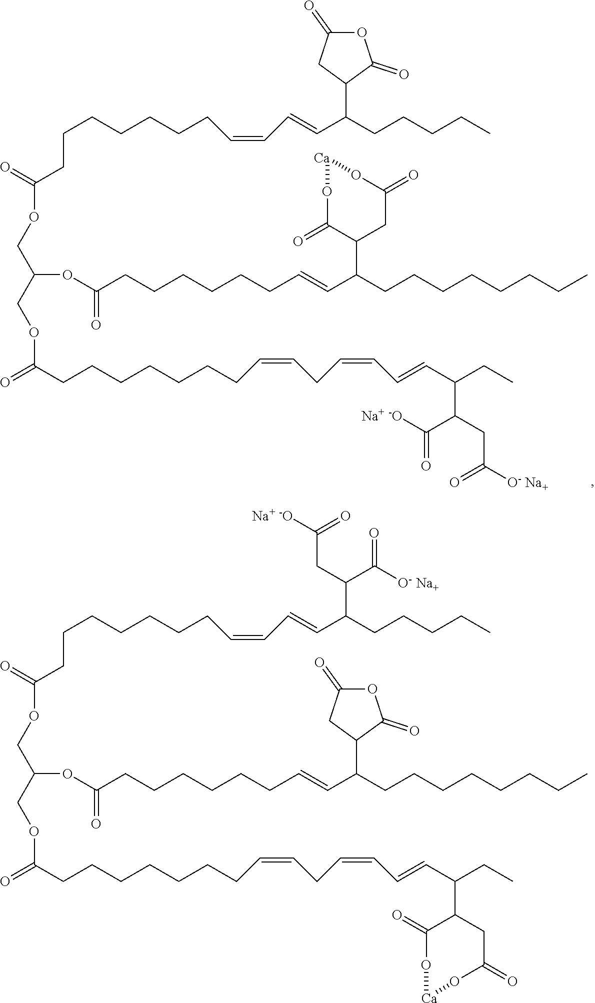

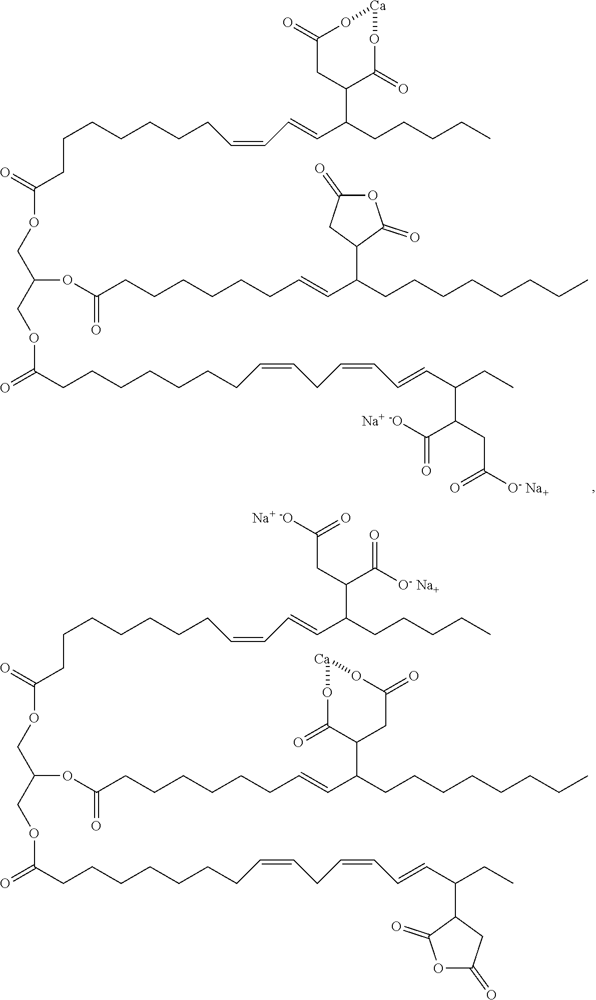

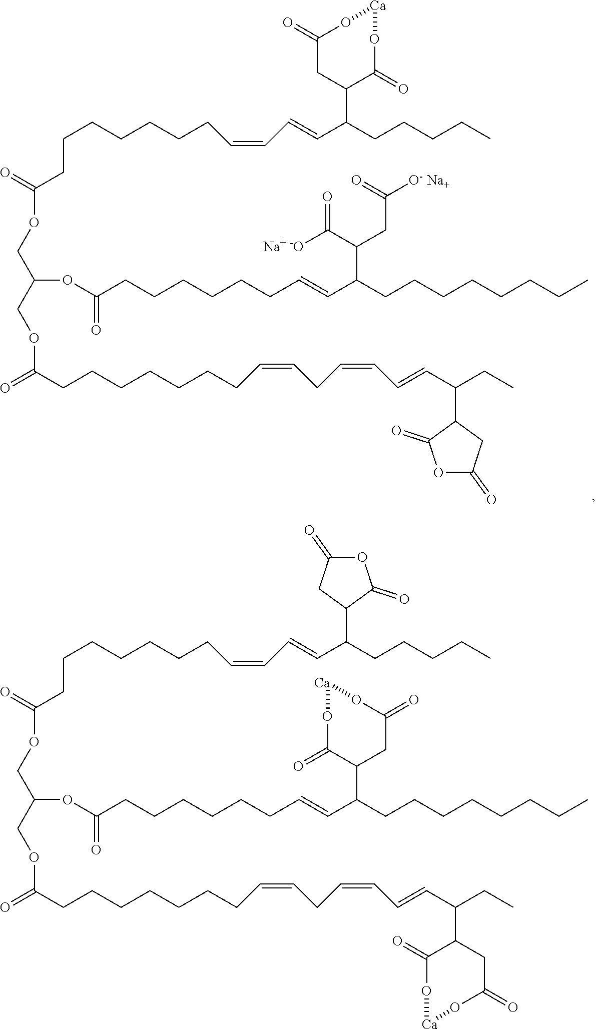

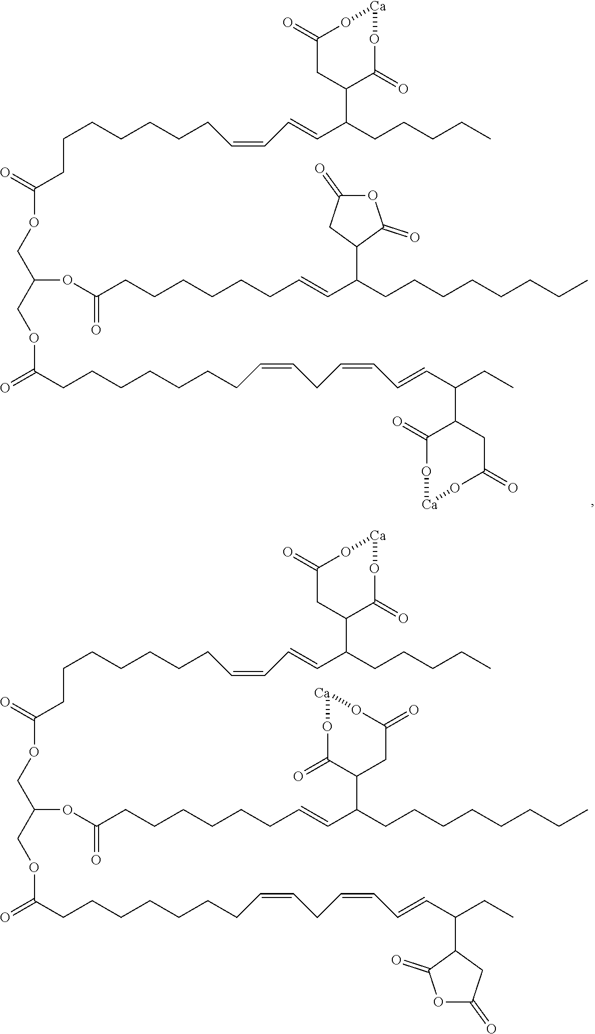

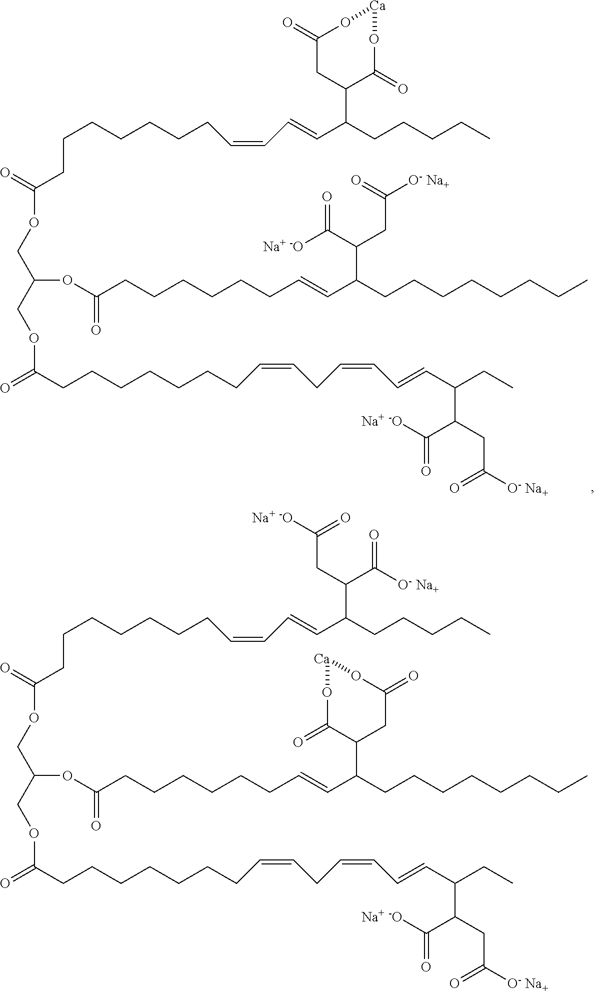

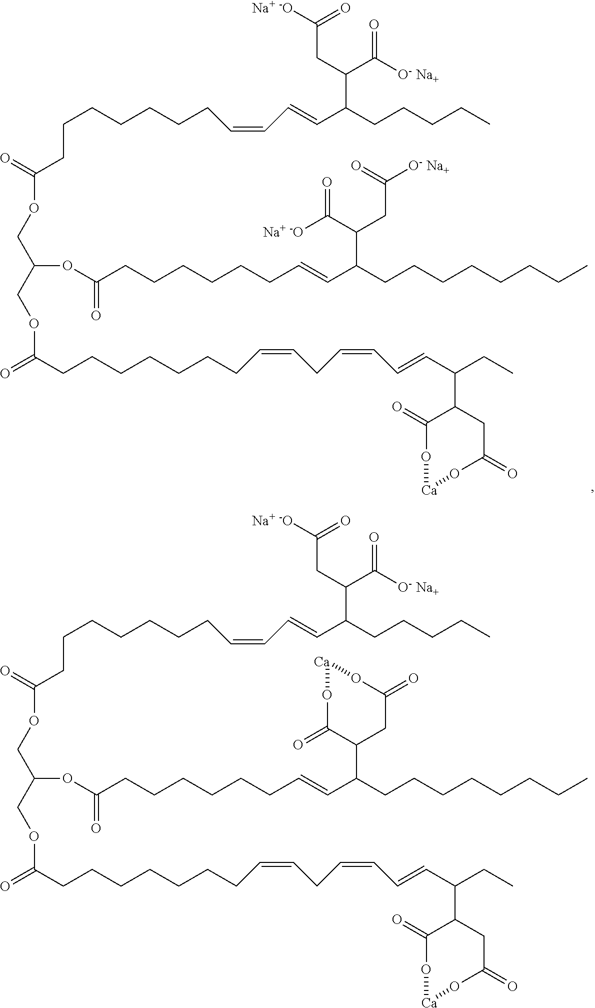

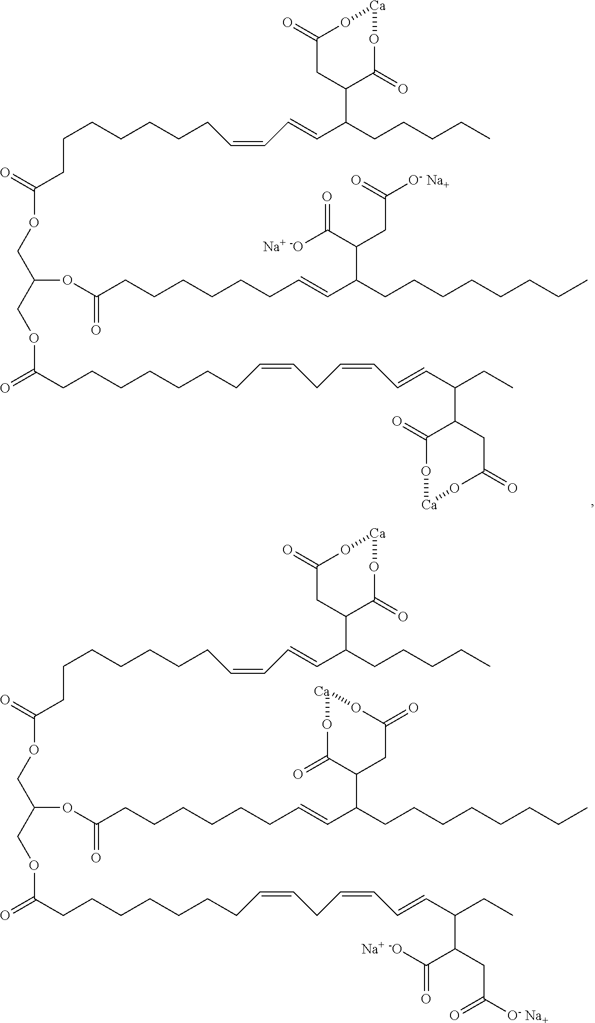

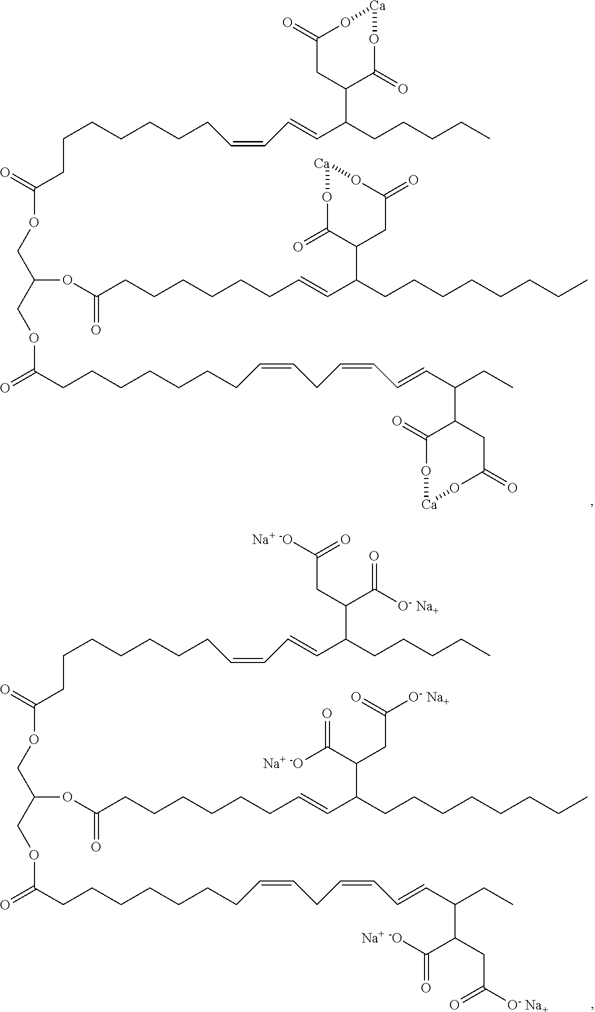

and combinations thereof.

In one non-limiting embodiment, the invention provides an agricultural composition comprising a reaction product of (A) a maleated natural oil, comprising a natural oil with maleated functionality; (B) a base; wherein the maleated functionality has been partially reacted with the base; and (C) a functionalized or unfunctionalized moiety selected from the group consisting of hydrophobic moieties, hydrophilic moieties, and combinations thereof; wherein the maleated functionality has been partially reacted with the functionalized or unfunctionalized moiety.

In one non-limiting embodiment, the invention provides an agricultural composition comprising a reaction product of (A) from about 0.01% to about 20.0% of a reaction product of (a) a maleated natural oil; and (b) a base; wherein the maleated functionality has been partially reacted with the base; and (c) a functionalized or unfunctionalized moiety selected from the group consisting of hydrophobic moieties, hydrophilic moieties, and combinations thereof wherein the maleated functionality has been partially reacted with the functionalized or unfunctionalized moiety; and (B) from about 1.0. to about 90.0 wt. % of one or more agriculturally active ingredient; and (C) from about 1 to about 99.0 wt. % of one or more ingredient.

In one non-limiting embodiment, the invention provides an agricultural composition comprising a reaction product of (A) from about 0.01 to about 20.0 wt. % of the reaction product comprises: (a) of a maleated natural oil, comprising a natural oil with maleated functionality; (b) a base selected from the group consisting of sodium hydroxide, potassium hydroxide, calcium hydroxide and mixtures thereof, wherein the maleated functionality has been partially reacted with the base; and (c) octyl dodecyl alcohol or a glycerol and their combinations thereof; wherein the maleated functionality has been partially reacted with the octyl dodecyl alcohol or a glycerol moiety; (B) from about 1.0 to about 90.0 wt. % of one or more agricultural active ingredient; and (C) from about 1.0 to about 99.0% of one or more additional ingredient.

In one non-limiting embodiment, the invention provides an agricultural composition comprising a reaction product of (a) of a maleated natural oil, comprising a natural oil with maleated functionality; (b) a base selected from the group consisting of sodium hydroxide, calcium hydroxide and mixtures thereof, wherein the maleated functionality has been partially reacted with the base; and (c) octyl dodecyl alcohol or a glycerol consisting of the following structures:

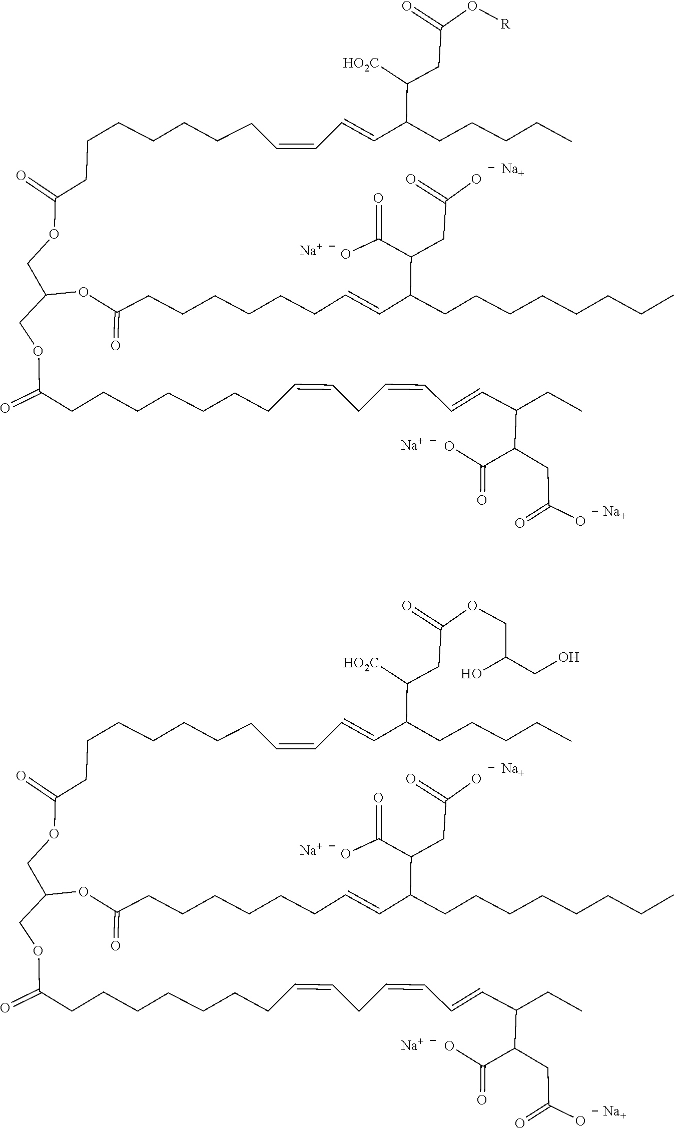

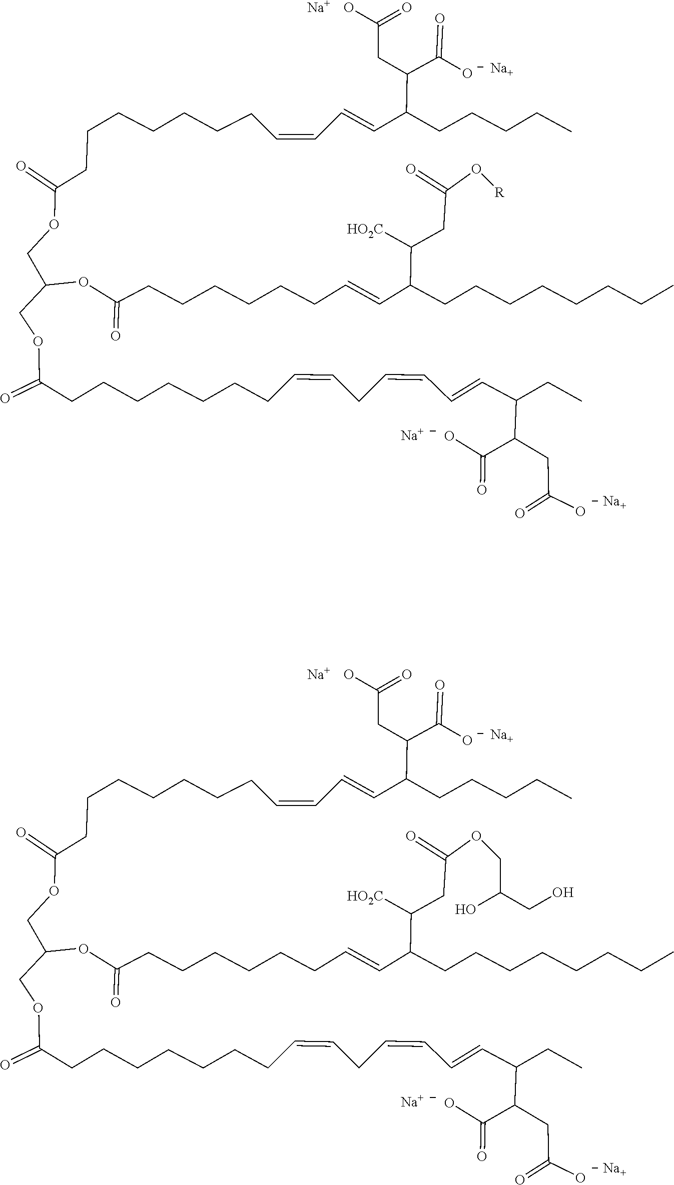

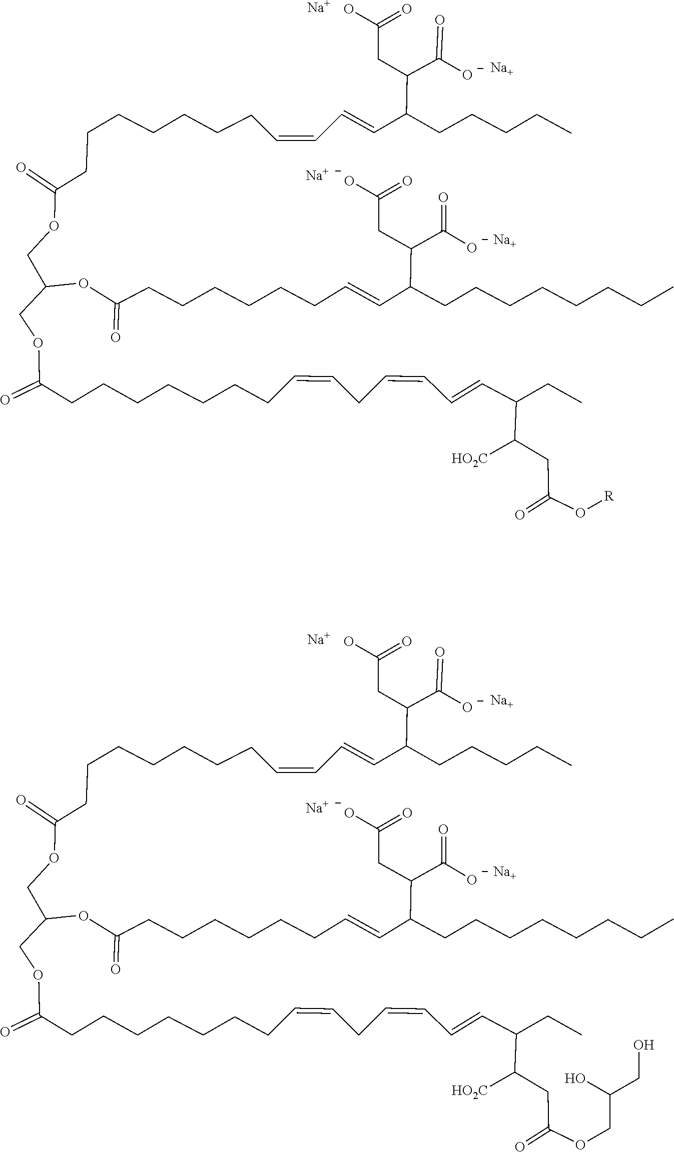

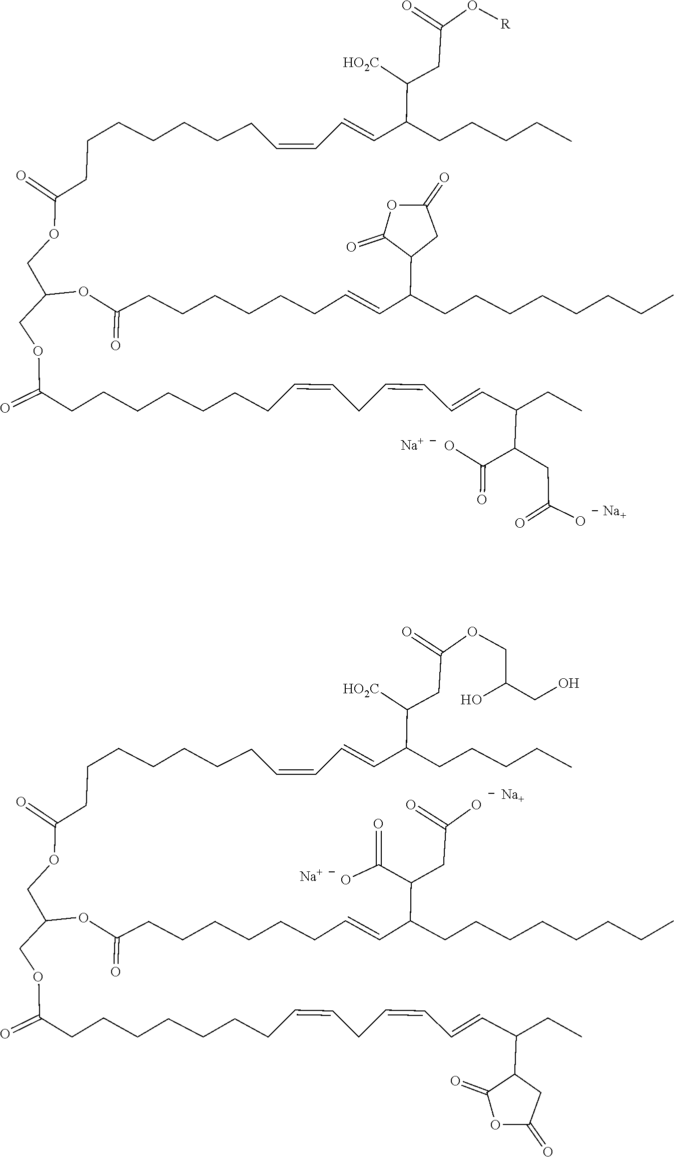

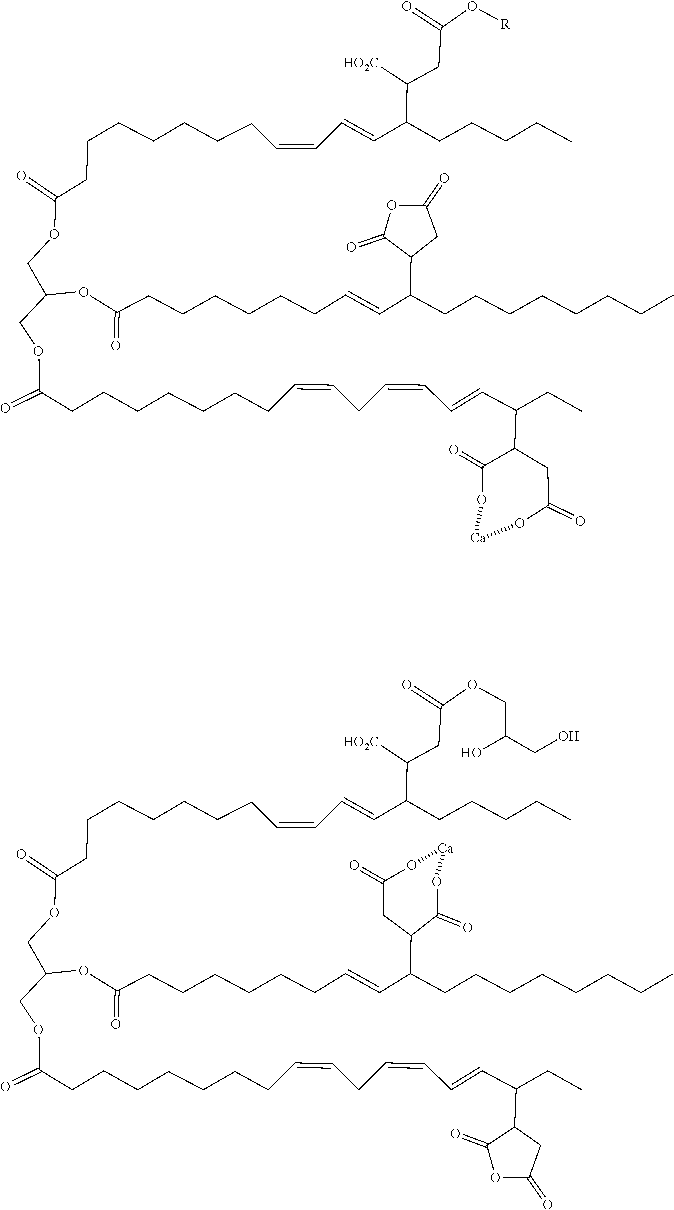

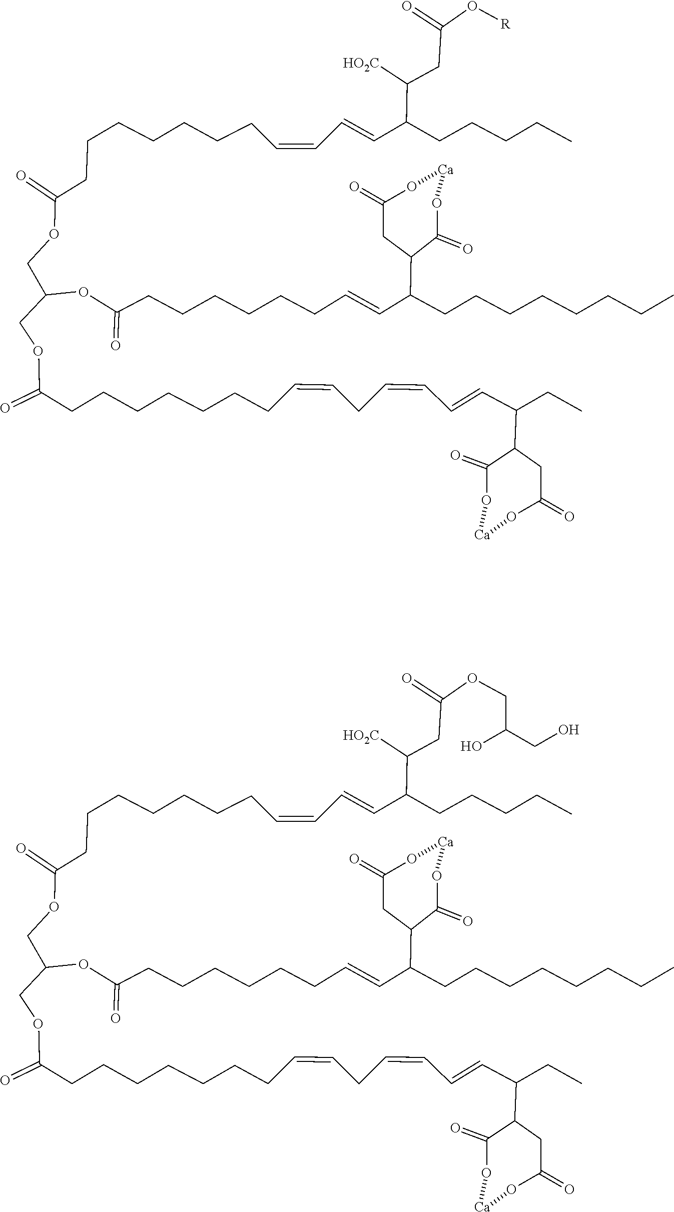

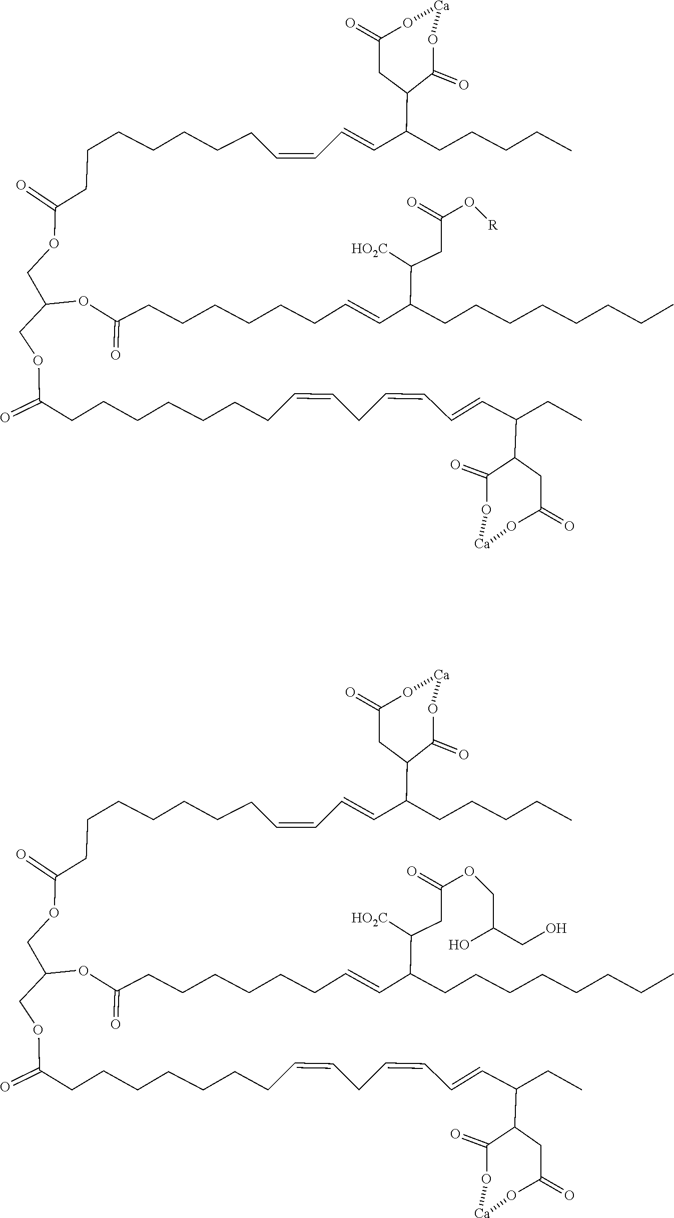

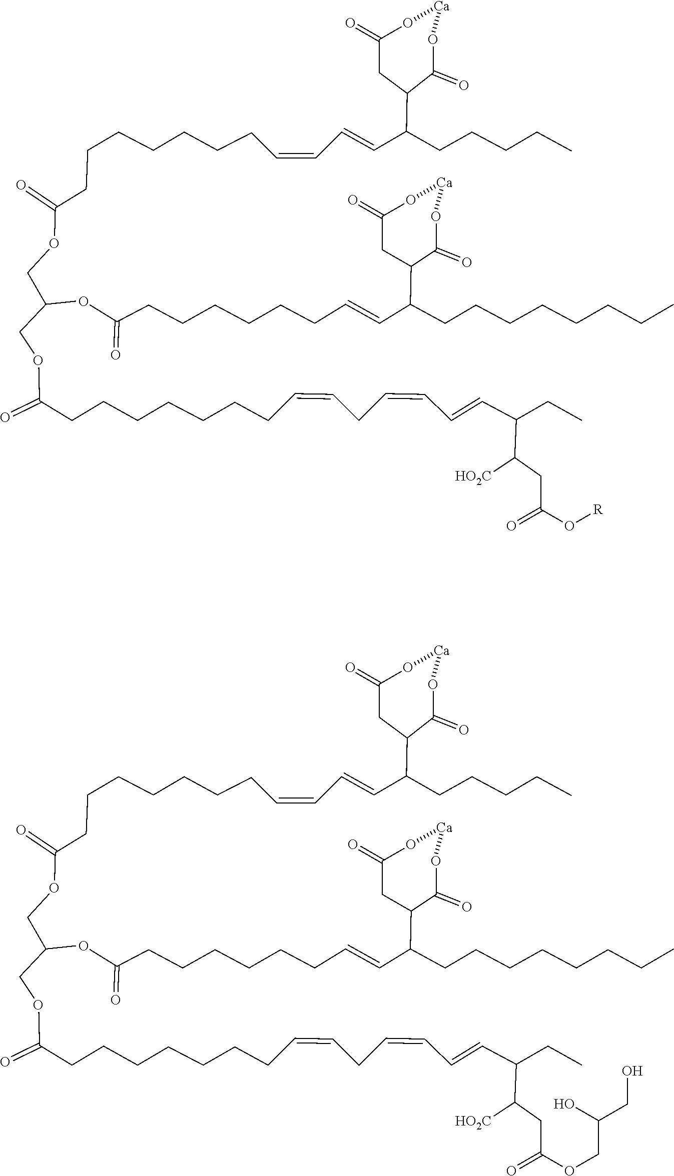

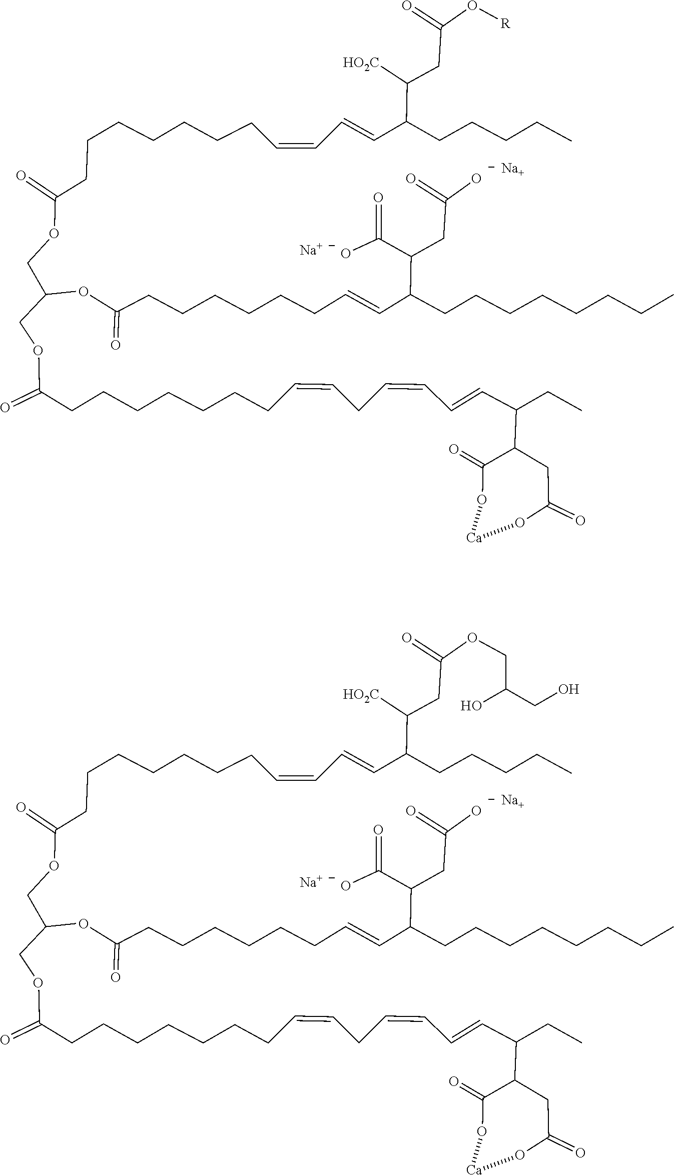

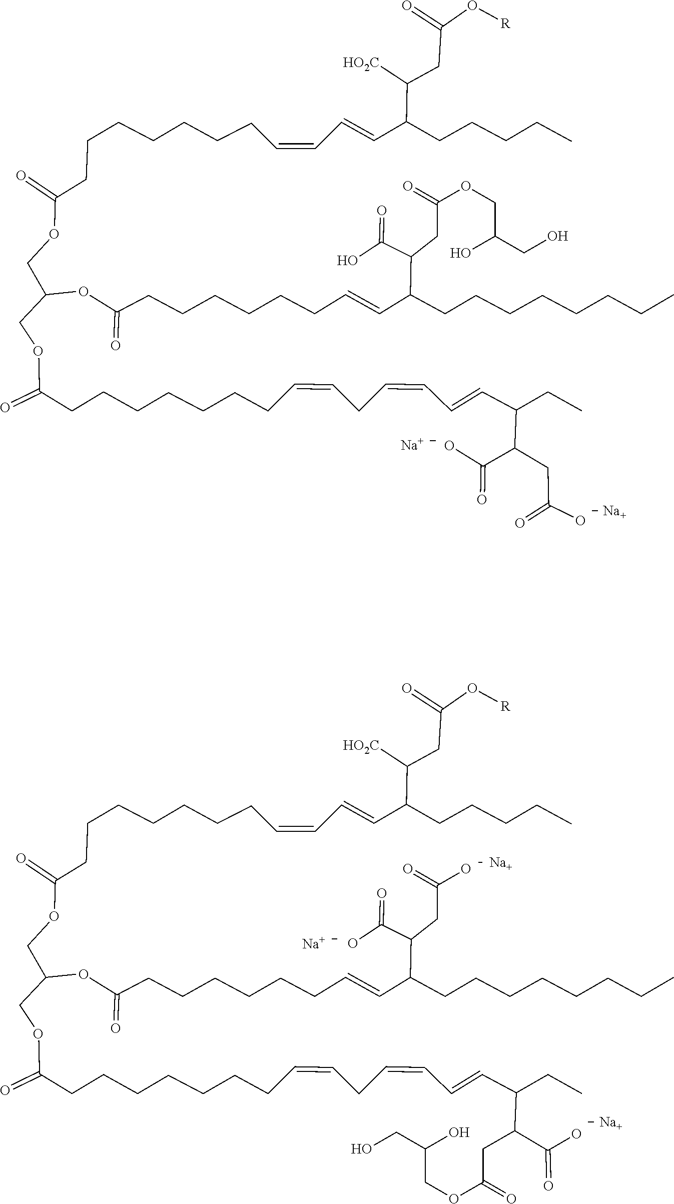

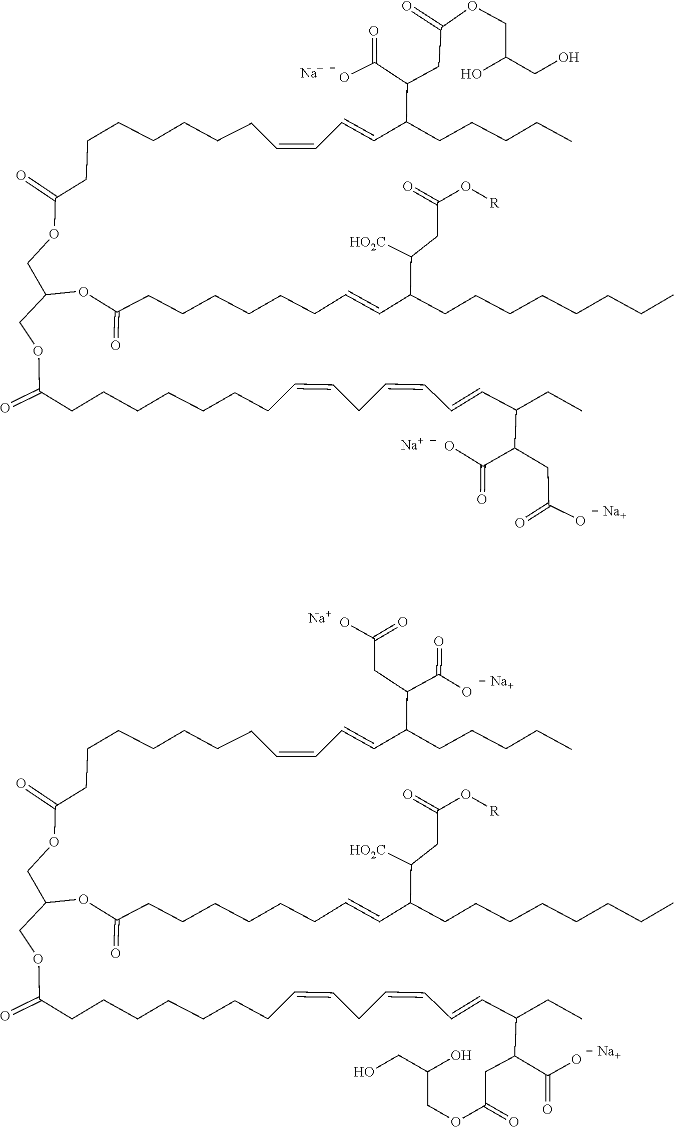

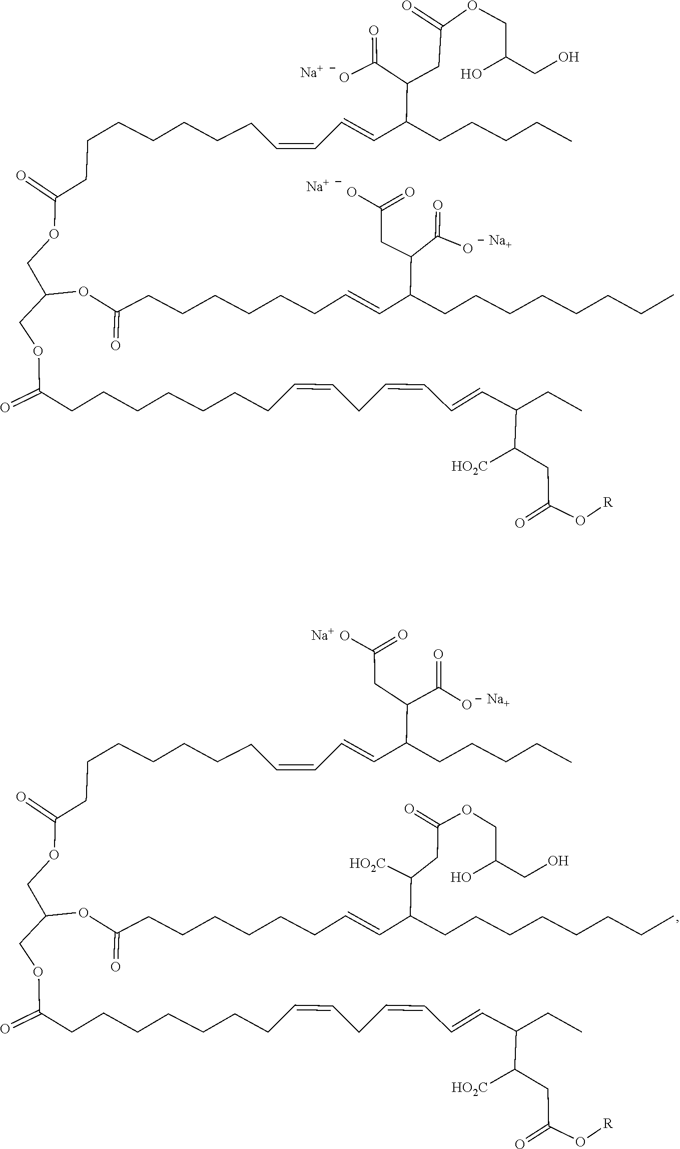

and combinations thereof, wherein R is ethyl, butyl, hexyl, octyl, 2-ethylhex-1-yl, 2-butyloct-1-yl, 2-hexyldec-1-yl, 2-octyldodec-1-yl or mixtures thereof.

In one non-limiting embodiment, the invention provides an composition comprising a reaction product of: (A) a maleated natural oil, comprising a natural oil with maleated functionality; and (B) a functionalized or unfunctionalized moiety selected from the group consisting of hydrophobic moieties, hydrophilic moieties, and combinations thereof, wherein the maleated functionality has been partially reacted with the functionalized or unfunctionalized moiety; and (C) a base, where in the reaction product is of

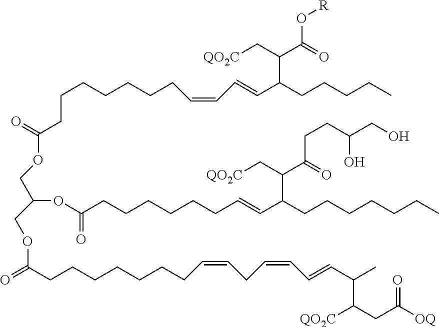

wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and wherein each Q is independently selected from the group consisting of Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

In one non-limiting embodiment, the invention provides an composition comprising a reaction product of: (A) a maleated natural oil, comprising a natural oil with maleated functionality; and (B) a functionalized or unfunctionalized moiety selected from the group consisting of hydrophobic moieties, hydrophilic moieties, and combinations thereof, wherein the maleated functionality has been partially reacted with the functionalized or unfunctionalized moiety; and (C) a base, where in the reaction product is a polymer of

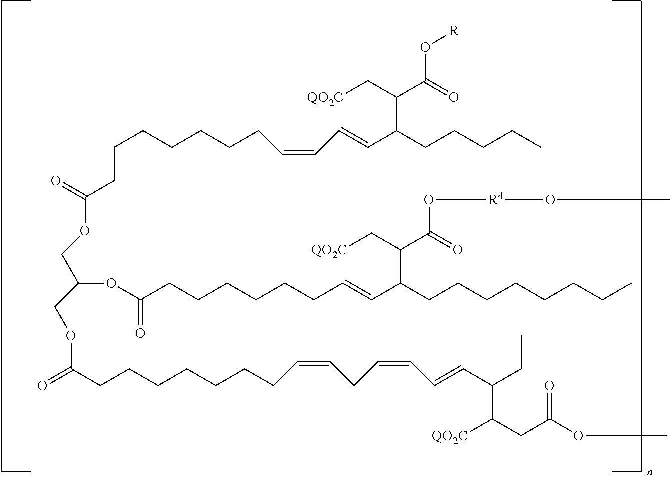

wherein n is greater than 1; wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

In one non-limiting embodiment, the invention provides an composition can be contemplated to applications selected from the group consisting of skin care compositions, oral care compositions, hair care compositions, energy compositions, lithium-ion battery compositions, construction compositions, biocidal compositions, preservative compositions, nutraceutical compositions, food compositions, agricultural compositions, coating compositions, ink compositions, cosmetic compositions, homecare compositions, industrial and institutional compositions, metal working compositions, textile compositions, laundry compositions, cleaning compositions, and disinfecting compositions.

In one non-limiting embodiment, the invention provides an agricultural composition comprising a reaction product of: (A) a maleated natural oil, comprising a natural oil with maleated functionality; and (B) a functionalized or unfunctionalized moiety selected from the group consisting of hydrophobic moieties, hydrophilic moieties, and combinations thereof, wherein the maleated functionality has been partially reacted with the functionalized or unfunctionalized moiety; and (C) a base, where in the reaction product is of

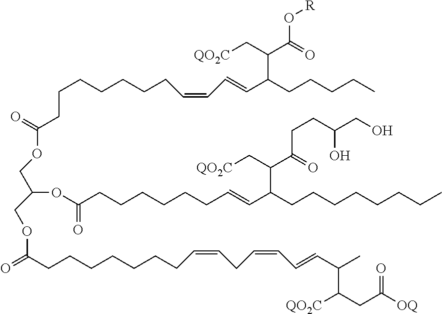

wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and wherein each Q is independently selected from the group consisting of Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

In one non-limiting embodiment, the invention provides an agricultural composition comprising a reaction product of: (A) a maleated natural oil, comprising a natural oil with maleated functionality; and (B) a functionalized or unfunctionalized moiety selected from the group consisting of hydrophobic moieties, hydrophilic moieties, and combinations thereof, wherein the maleated functionality has been partially reacted with the functionalized or unfunctionalized moiety; and (C) a base, where in the reaction product is a polymer of

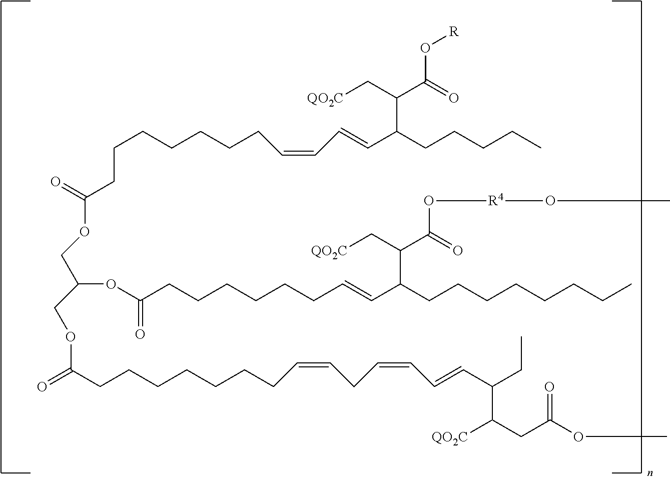

wherein n is greater than 1; wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

In one non-limiting embodiment, the invention provides an agricultural composition for topical application to an animal or a plant comprising a safe and effective amount of (A) from about 0.01 to about 20.0 wt. % of the reaction product of

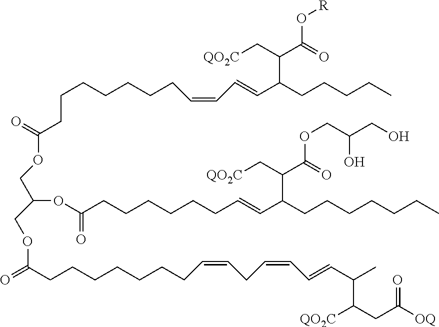

wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and wherein each Q is independently selected from the group consisting of Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium; (B) from about 1.0 to about 90.0 wt. % of one or more agriculturally active ingredient; and (C) from about 1.0 to about 99.0 wt. % of one or more additional ingredient.

In one non-limiting embodiment, the invention provides an agricultural composition for topical application to an animal or a plant comprising a safe and effective amount of (A) from about 0.01 to about 20.0 wt. % of the reaction product is of

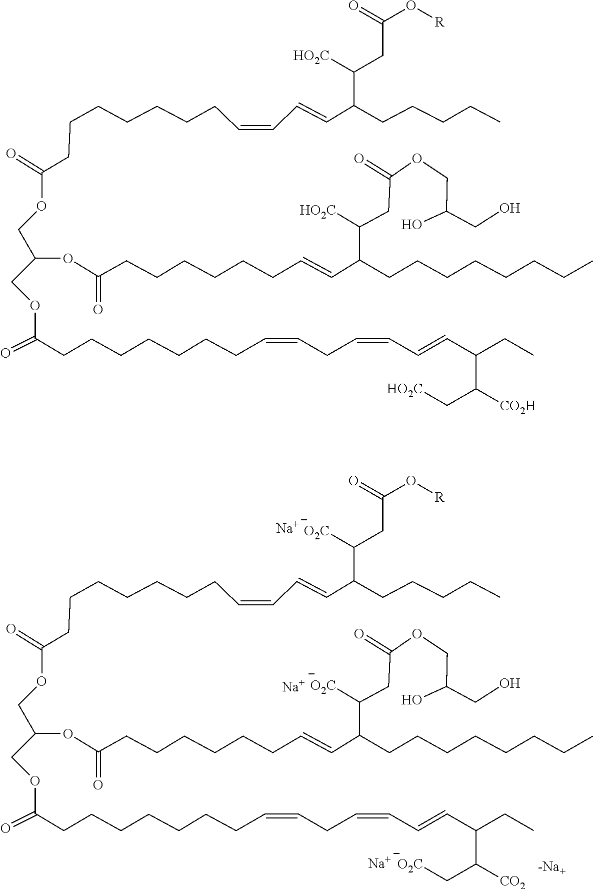

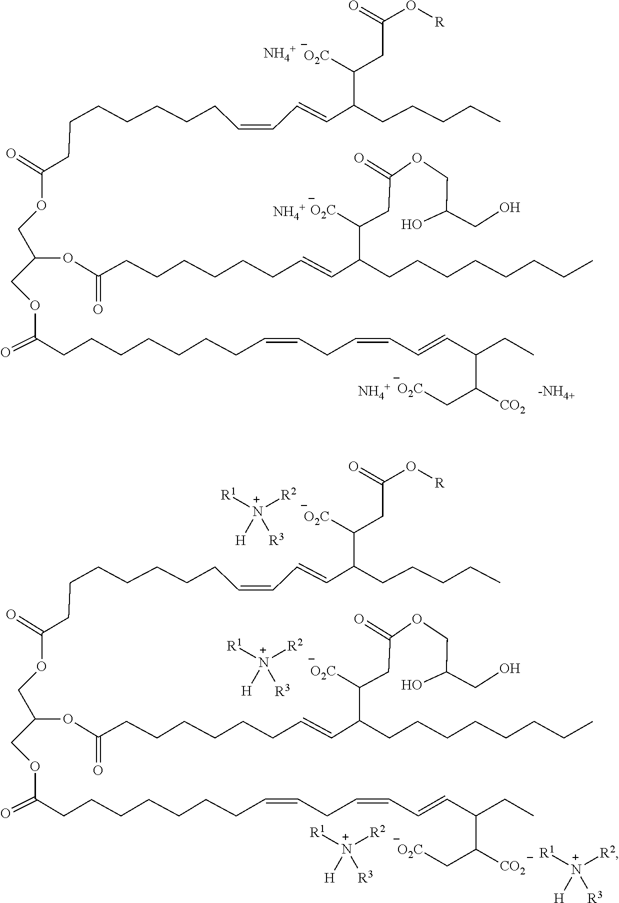

wherein R is ethyl, butyl, hexyl, octyl, 2-ethylhex-1-yl, 2-butyloct-1-yl, 2-hyxyldec-1-yl, 2-octyldodec-1-yl, or mixtures thereof; and wherein R¹, R² and R³ are each, independently, hydrogen, methyl, or an unsubstituted or substituted alkyl group containing 2 to 18 carbon atoms and, optionally, containing heteroatoms; (B) from about 1.0 to about 90.0 wt. % of one or more agriculturally active ingredient; and (C) from about 1.0 to about 99.0 wt. % of one or more additional ingredient.

In one non-limiting embodiment, the invention provides an agricultural composition for topical application to an animal or a plant comprising a safe and effective amount of (A) from about 0.01 to about 20.0 wt. % of the reaction product is a polymer of

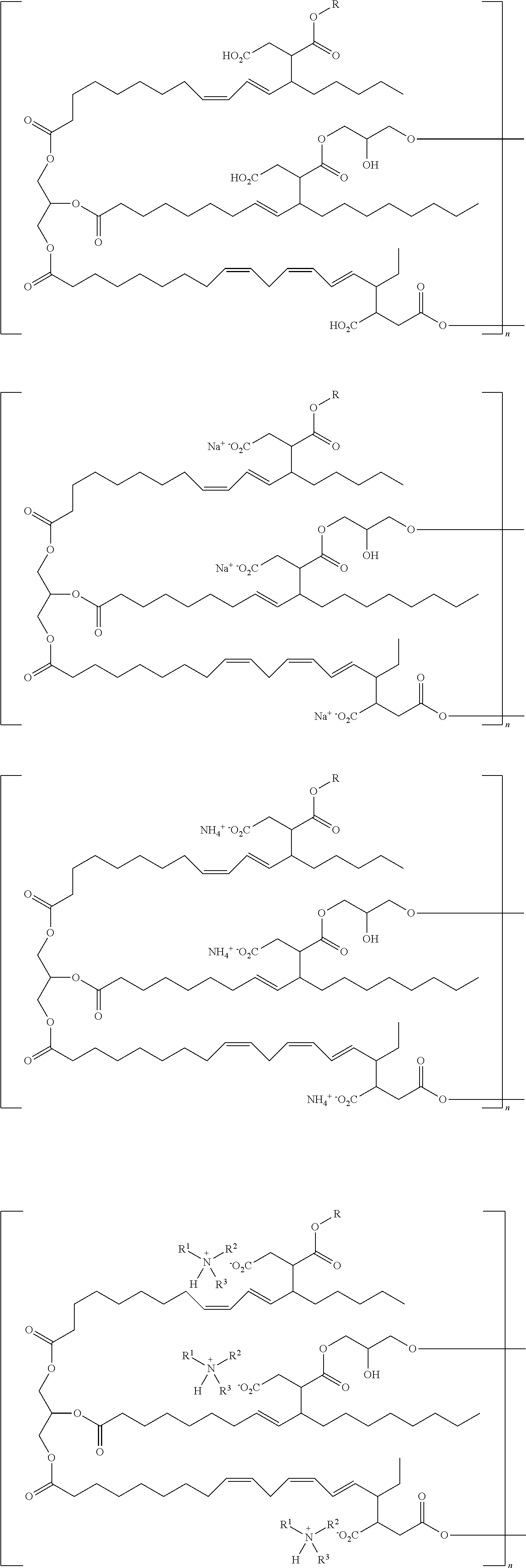

wherein 1<n<30; wherein R is ethyl, butyl, hexyl, octyl, 2-ethylhex-1-yl, 2-butyloct-1-yl, 2-hyxyldec-1-yl, 2-octyldodec-1-yl, or mixtures thereof; wherein R¹, R² and R³ are each, independently, hydrogen, methyl, or an unsubstituted or substituted alkyl group containing 2 to 18 carbon atoms and, optionally, containing heteroatoms; and wherein R⁴ is

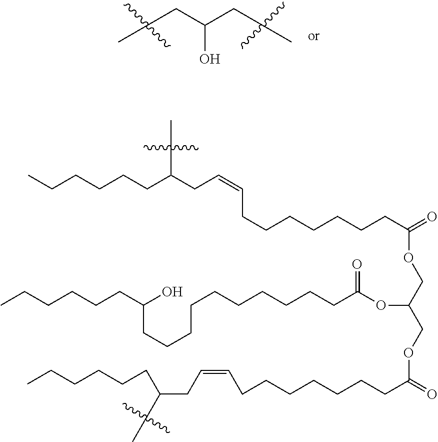 [SMILES: *CC(O)C* ; *C(C/C=C\CCCCCCCC(=O)OCC(COC(=O)CCCCCCC/C=C\CC(*)CCCCCC)OC(=O)CCCCCCCCCCC(O)CCCCCC)CCCCCC]

or a mixture thereof; (B) from about 1.0 to about 90.0 wt. % of one or more agriculturally active ingredient; and (C) from about 1.0 to about 99.0 wt. % of one or more additional ingredient.

In one non-limiting embodiment, the invention provides an agricultural composition selected from the group consisting of an adjuvant composition, a fertilizer composition, a nutrient composition, a plant strengthener composition, a seed coating composition, a soil conditioner composition, a livestock composition, a granular composition, a controlled release composition, a film coating composition, a pesticide composition selected from the group of rodenticide, insecticide, miticide, algicide, molluscicide, acaricide, avicide, fungicide and herbicide compositions, a germicide composition, an antibiotic composition, an antibacterial composition, an antiviral composition, an antifungal composition, an antiprotozoal composition, an anti-parasite composition, a wood preservation composition, an antimicrobial composition or a yield enhancer composition to soil for the plant.

In one non-limiting embodiment, the invention provides a seed coating composition comprising (A) from about 0.01% to about 20.0% a reaction product of a maleated natural oil; base and a functionalized or unfunctionalized moiety selected from the group consisting of hydrophobic moieties, hydrophilic moieties, and combinations thereof, (B) from about 1.0% to about 90.0% one or more agricultural active ingredient; and (C) from about 1.0% to about 99.0% one or more additional ingredient.

In one non-limiting embodiment, the invention provides a livestock composition comprising (A) from about 0.01% to about 20.0% a reaction product of a maleated natural oil and base; (B) from about 1.0% to about 90.0% one or more agricultural active ingredient(s); and (C) from about 1.0% to about 99.0% one or more additional ingredient.

In one non-limiting embodiment, the invention provides a granular composition comprising (A) from about 0.01% to about 20.0% a reaction product of a maleated natural oil and base; (B) from about 1.0% to about 90.0% one or more agriculturally active ingredient; and (C) from about 1.0% to about 99.0% one or more additional ingredient.

In one non-limiting embodiment, the invention provides a controlled release composition comprising (A) from about 0.01% to about 20.0% a reaction product of a maleated natural oil and base; (B) from about 1.0% to about 90.0% one or more agriculturally active ingredient(s); and (C) from about 1.0% to about 99.0% one or more additional ingredient.

In one non-limiting embodiment, the invention provides a film coating composition comprising (A) from about 0.01% to about 20.0% a reaction product of a maleated natural oil and base; (B) from about 1.0% to about 90.0% one or more agriculturalyl active ingredient(s); and (C) from about 1.0% to about 99.0% one or more additional ingredient.

In one non-limiting embodiment, the invention provides a seed coating composition comprising (A) from about 0.01% to about 20.0% a reaction product of a maleated natural oil; base and a functionalized or unfunctionalized moiety selected from the group consisting of hydrophobic moieties, hydrophilic moieties, and combinations thereof, (B) from about 1.0% to about 90.0% one or more agriculturally active ingredient; and (C) from about 1.0% to about 99.0% one or more additional ingredient.

In one non-limiting embodiment, the invention provides a livestock composition comprising (A) from about 0.01% to about 20.0% a reaction product of a maleated natural oil; base and a functionalized or unfunctionalized moiety selected from the group consisting of hydrophobic moieties, hydrophilic moieties, and combinations thereof, (B) from about 1.0% to about 90.0% one or more agriculturally active ingredient; and (C) from about 1.0% to about 99.0% one or more additional ingredient.

In one non-limiting embodiment, the invention provides a granular composition comprising (A) from about 0.01% to about 20.0% a reaction product of a maleated natural oil; base and a functionalized or unfunctionalized moiety selected from the group consisting of hydrophobic moieties, hydrophilic moieties, and combinations thereof, (B) from about 1.0% to about 90.0% one or more agriculturally active ingredient; and (C) from about 1.0% to about 99.0% one or more additional ingredient.

In one non-limiting embodiment, the invention provides a controlled release composition comprising (A) from about 0.01% to about 20.0% a reaction product of a maleated natural oil; base and a functionalized or unfunctionalized moiety selected from the group consisting of hydrophobic moieties, hydrophilic moieties, and combinations thereof, (B) from about 1.0% to about 90.0% one or more agriculturally active ingredient; and (C) from about 1.0% to about 99.0% one or more additional ingredient.

In one non-limiting embodiment, the invention provides a film coating composition comprising (A) from about 0.01% to about 20.0% a reaction product of a maleated natural oil; base and a functionalized or unfunctionalized moiety selected from the group consisting of hydrophobic moieties, hydrophilic moieties, and combinations thereof, (B) from about 1.0% to about 90.0% one or more agriculturally active ingredient; and (C) from about 1.0% to about 99.0% one or more additional ingredient.

In one non-limiting embodiment, the invention provides a seed coating composition comprising (A) from about 0.01% to about 20.0% a reaction product of

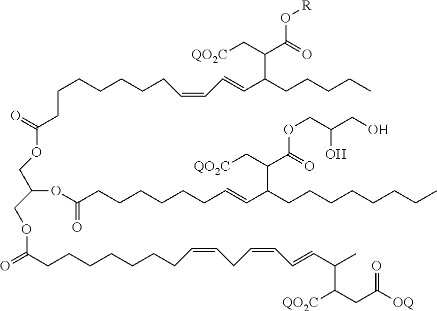

wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and wherein each Q is independently selected from the group consisting of Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium; (B) from about 1.0% to about 90.0% one or more agricultural active ingredient; and (C) from about 1.0% to about 99.0% one or more additional ingredient.

In one non-limiting embodiment, the invention provides a livestock composition comprising (A) from about 0.01% to about 20.0% a reaction product of

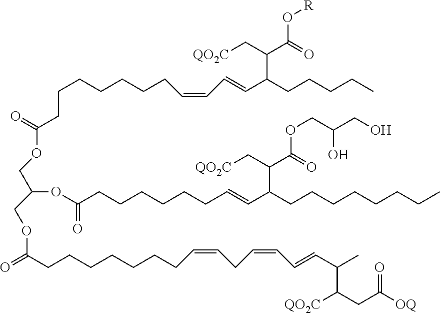

wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and wherein each Q is independently selected from the group consisting of Li, Na, K, Rb, Cs, Fr, 2 Mg, ½ Ca, ½ Co, ½ Cu, ½ Zn, ammonium, alkylammonium, dialkylammonium, trialkylammonium and tetraalkylammonium; (B) from about 1.0% to about 90.0% one or more agricultural active ingredient; and (C) from about 1.0% to about 99.0% one or more additional ingredient.

In one non-limiting embodiment, the invention provides a granular composition comprising (A) from about 0.01% to about 20.0% a reaction product of

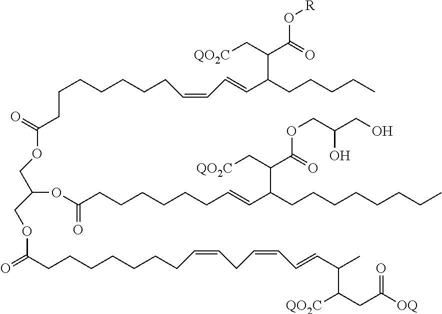

wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and wherein each Q is independently selected from the group consisting of Li, Na, K, Rb, Cs, Fr, 2 Mg, ½ Ca, ½ Co, ½ Cu, ½ Zn, ammonium, alkylammonium, dialkylammonium, trialkylammonium and tetraalkylammonium; (B) from about 1.0% to about 90.0% one or more agricultural active ingredient; and (C) from about 1.0% to about 99.0% one or more additional ingredient.

In one non-limiting embodiment, the invention provides a controlled release composition comprising (A) from about 0.01% to about 20.0% a reaction product of

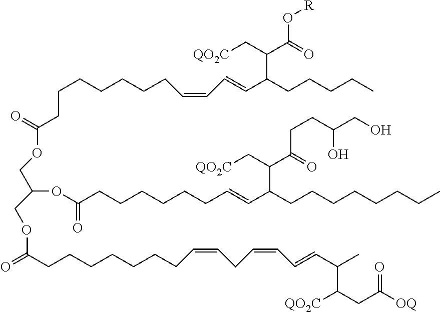

wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and wherein each Q is independently selected from the group consisting of Li, Na, K, Rb, Cs, Fr, 2 Mg, 1 Ca, ½ Co, ½ Cu, ½ Zn, ammonium, alkylammonium, dialkylammonium, trialkylammonium and tetraalkylammonium; (B) from about 1.0% to about 90.0% one or more agricultural active ingredient; and (C) from about 1.0% to about 99.0% one or more additional ingredient.

In one non-limiting embodiment, the invention provides a film coating composition comprising (A) from about 0.01% to about 20.0% a reaction product of

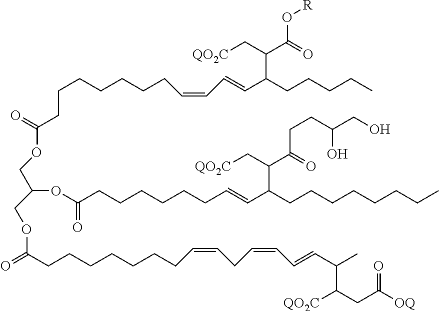

wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and wherein each Q is independently selected from the group consisting of Li, Na, K, Rb, Cs, Fr, 2 Mg, 2 Ca, ½ Co, ½ Cu, ½ Zn, ammonium, alkylammonium, dialkylammonium, trialkylammonium and tetraalkylammonium; (B) from about 1.0% to about 90.0% one or more agricultural active ingredient; and (C) from about 1.0% to about 99.0% one or more additional ingredient.

In one non-limiting embodiment, the invention provides a seed coating composition comprising (A) from about 0.01% to about 20.0% a reaction product is a polymer of

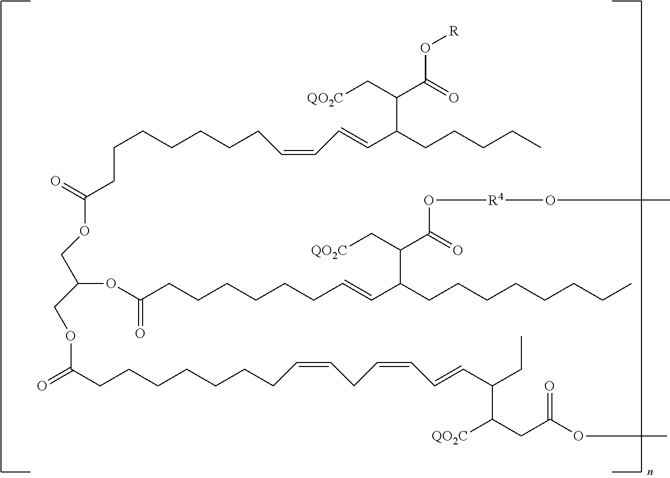

wherein n is greater than 1; wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium; (B) from about 1.0% to about 90.0% one or more agricultural active ingredient; and (C) from about 1.0% to about 99.0% one or more additional ingredient.

In one non-limiting embodiment, the invention provides a livestock composition comprising (A) from about 0.01% to about 20.0% a reaction product is a polymer of

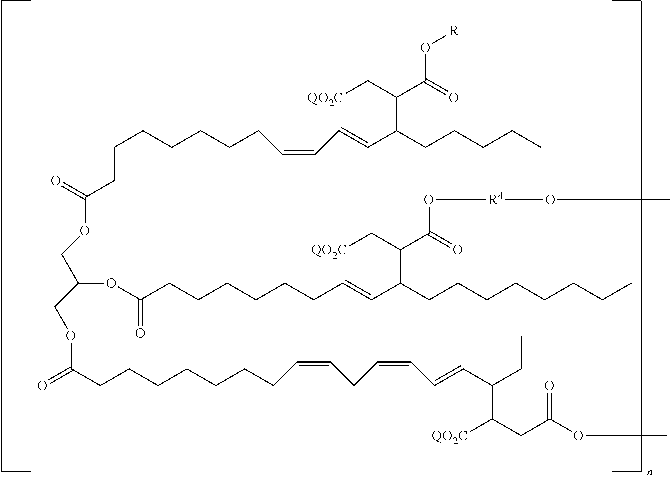

wherein n is greater than 1; wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium; (B) from about 1.0% to about 90.0% one or more agricultural active ingredient; and (C) from about 1.0% to about 99.0% one or more additional ingredient.

In one non-limiting embodiment, the invention provides a granular composition comprising (A) from about 0.01% to about 20.0% a reaction product is a polymer of

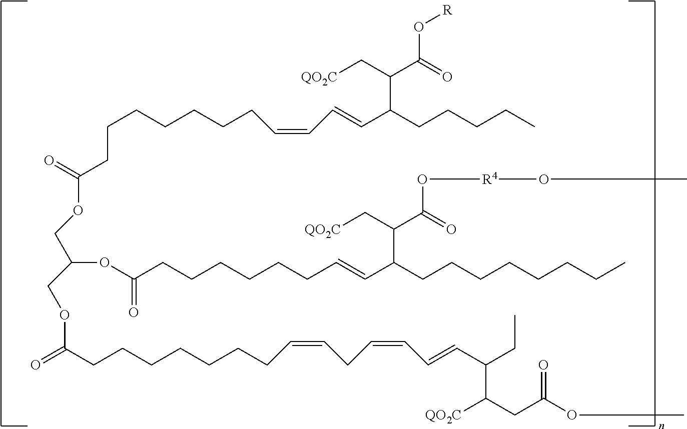

wherein n is greater than 1; wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium; (B) from about 1.0% to about 90.0% one or more agricultural active ingredient; and (C) from about 1.0% to about 99.0% one or more additional ingredient.

In one non-limiting embodiment, the invention provides a controlled release composition comprising (A) from about 0.01% to about 20.0% a reaction product is a polymer of

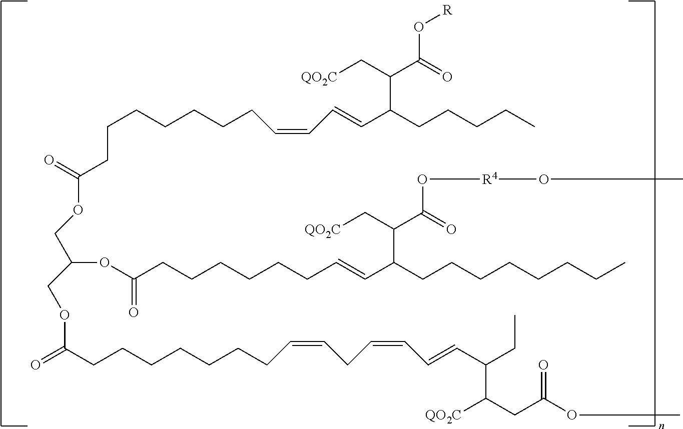

wherein n is greater than 1; wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium; (B) from about 1.0% to about 90.0% one or more agricultural active ingredient; and (C) from about 1.0% to about 99.0% one or more additional ingredient.

In one non-limiting embodiment, the invention provides a film coating composition comprising (A) from about 0.01% to about 20.0% a reaction product is a polymer of

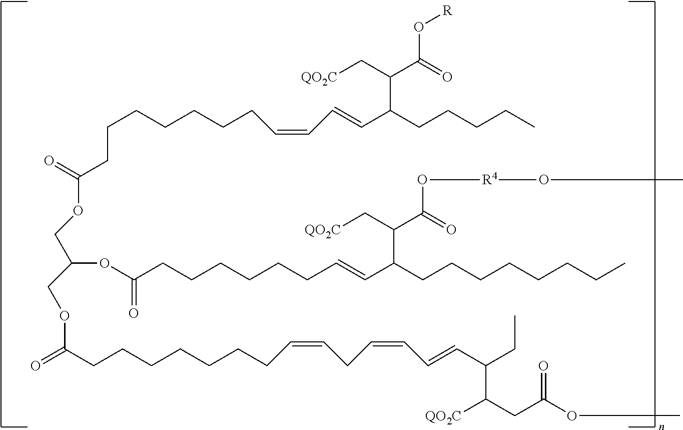

wherein n is greater than 1; wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium; (B) from about 1.0% to about 90.0% one or more agricultural active ingredient; and (C) from about 1.0% to about 99.0% one or more additional ingredient.

According to another embodiment of the present application, it is contemplated to employ at least one agricultural active ingredient selected from the group consisting of fertilizers, herbicides, ovicides, fungicides, pesticides selected from the group of rodenticides, miticides, algicides, molluscicides, acaricides, avicides, insecticides, germicides, antibiotics, antibacterial, antivirals, antifungals, antiprotozoals, anti-parasites and antimicrobials compounds.

According to another embodiment of the present application, pesticides contemplated for use in the present invention include, fungicides, herbicides, insecticides, miticides, nematicides, acaricides, and molluscicides.

The fertilizer contemplated for use in the present application can include an inorganic fertilizer, a nitrogen fertilizer, a potassium fertilizer, a phosphate fertilizer, an organic fertilizer, a manure, a compost, a rock phosphate, a bone meal, an alfalfa, a wood chip, a langbeinite, a cover crops, potassium sulfate, a rock powder, ash, a blood meal, a fish meal, a fish emulsion, an algae, a chitosan and a molasse.

Examples of herbicides contemplated for use in the present application can include phenoxy carboxylic acids (e.g. 2,4-D-acid, MCPA), benzoic acids (e.g. Dicamba-acid), ureas, (e.g. Diuron) sulfonylureas (e.g. methylsulfuron-methyl, rimsulfuron), triazines (e.g. atrazine, simazine, ameztryn), triazolinones (e.g. amicarbazone) and pyridine carboxylic acids (e.g. triclopyr).

Examples of fungicides contemplated for use in the present application include fungicides of the classes triazoles (e.g. tebuconazole, tetraconazole, cyproconazole, epoxiconazole, difenconazole, propiconazole, prothioconazole, myclobutanil), strobilurins (e.g. trifloxystrobin, azoxystrobin, fluoxastrobin, pyraclostrobin), alkylenebis(dithiocarbamate) compounds (e.g. mancozeb) and benzimidazoles (e.g carbendazim).

Examples of insecticides contemplated for use in the present invention include neonicotinoids (e.g. thiamethoxam, clothianidin, thiacloprid, dinotefuran, acetamiprid, nitenpyram, imidacloprid), amidines (e.g. amitraz), organophosphates (e.g. chlorpyrifos) and pyrethroids (e.g. permethrin, bifenthrin, deltamethrin).

Antimicrobials suitable for agrochemical compositions according to the present invention includes germicides, antibiotics, antibacterials, antivirals, antifungals, antiprotozoals and antiparasites. Bactericidal disinfectants mostly used are those applying active chlorine (i.e., hypochlorites, chloramines, dichloroisocyanurate and trichloroisocyanurate, wet chlorine, chlorine dioxide, etc.), active oxygen (peroxides such as peracetic acid, potassium persulfate, sodium perborate, sodium percarbonate and urea perhydrate), iodine (iodpovidone (povidone-iodine, Betadine), Lugol's solution, iodine tincture, iodinated nonionic surfactants), concentrated alcohols (mainly ethanol, 1-propanol, called also n-propanol and 2-propanol, called isopropanol and mixtures thereof, further, 2-phenoxyethanol and 1- and 2-phenoxypropanols are used), phenolic substances (such as phenol (also called “carbolic acid”), cresols (called “Lysole” in combination with liquid potassium soaps), halogenated (chlorinated, brominated) phenols, such as hexachlorophene, triclosan, trichlorophenol, tribromophenol, pentachlorophenol, Dibromol and salts thereof), cationic surfactants such as some quaternary ammonium cations (such as benzalkonium chloride, cetyl trimethylammonium bromide or chloride, didecyldimethylammonium chloride, cetylpyridinium chloride, benzethonium chloride) and others, non-quarternary compounds such as chlorhexidine, glucoprotamine, octenidine dihydrochloride, etc.), strong oxidizers such as ozone and permanganate solutions; heavy metals and their salts such as colloidal silver, silver nitrate, mercury chloride, phenylmercury salts, copper sulfate, copper oxide-chloride etc. Heavy metals and their salts are the most toxic and environmentally hazardous bactericides and, therefore, their use is strongly suppressed or forbidden; further, also properly concentrated strong acids (phosphoric, nitric, sulfuric, amidosulfuric, toluenesulfonic acids) and alcalis (sodium, potassium, calcium hydroxides).

Suitable examples of plant growth regulators contemplated for use in the present invention include, but are not limited to, phosphonic acids (e.g. ethephon), gibberellins cytokinins (e.g. 6-benzylaminopurine), auxins (e.g. 1-naphtylacetic acid, triazoles, strobilurins, alkylenebis(dithiocarbamate) compounds, benzimidazoles, phenoxy carboxylic acids, benzoic acids, ureas, sulfonylureas, triazines, pyridine carboxylic acids, neonicotinides, amidines, organophosphates, and pyrethroids.

According to another embodiment of the present application, it is contemplated to employ at least one ingredient selected from the group consisting of adjuvant selected from the group of acidifying agents, buffering agents, anti-foam agents, defoaming agents, anti-transpirants, dyes and brighteners, compatibility agents, crop oil concentrates, oil surfactants, deposition agents, drift reduction agents, foam markers, feeding stimulants, herbicide safeners, spreaders, extenders, adhesive agents, suspension agents, gelling agents, synergists, wetting agents, emulsifiers, dispersing agents, penetrants, tank and equipment cleaners, neutralizers, water absorbents, water softeners and mixtures thereof.

According to another embodiment of the present application, it is contemplated to employ at least one ingredient further selected from the group consisting of solvents, liquid carriers, solid carriers or fillers, surfactants, solubilizers, penetration enhancers, protective colloids, thickeners, humectants, repellents, attractants, compatibilizers, bactericides, anti-freezing agents, crystallization inhibitors, colorants, tackifiers, binders, preservatives, pH adjuster, clarifiers, stabilizers, UV stabilizers, and mixtures thereof.

According to another embodiment of the present invention, an agricultural composition can include an acidifying agent that improves the efficacy of the agricultural composition. The acidifying agent can be suitable organic and inorganic acids selected from the group consisting of hydrochloric acid, nitric acid, acetic acid, phosphoric acid, polyphosphoric acid, perchloric acid, and combinations thereof.

According to another embodiment of the present invention, an agricultural composition can include a buffering agent to reduce the pH. A buffering agent includes mineral acids such as sulfuric acid, sulfonic acid, sulfurous acid, nitric acid, or nitrous acid; an organic acid such as glutaric acid, gluconic acid, lactic acid, glycolic acid, acrylic acid, or combinations thereof.

According to another embodiment of the present invention, an agricultural composition can comprise effective anti-transparent compounds reduces moisture loss. Antitranspirant compounds includes sorbitol, mannitol, and sucrose, silicones, polyethylene, polyvinyl chloride, paraffin, turpentine diene (terpene resin).

According to another embodiment of the present invention, an agricultural composition comprising a suitable “compatibility agent” which allows easier mixing of two or more components in a solution, thereby allowing the use of two or more chemicals in a tank that would otherwise be incompatible. Preferred compatibility agents are alkali metal acetates, earth alkali metal acetates, and mixtures thereof.

According to another embodiment of the present invention, an agricultural composition comprising a suitable crop oil concentrate (COCs) can be used to enhance the weed control efficacy, reduced volatility, lower water solubility, low surface tension, improved surface coverage and penetration efficacy of an agricultural composition. Crop oil concentrate (COCs) can be aromatic or paraffinic solvent, a fatty acid, one or more lower alkyl fatty acid esters, polyoxyalkylene sulfates, phosphates, or carboxylates.

According to another embodiment of the present invention, agricultural compositions can be utilize drift reduction agents as a soil or foliage penetrant, which can reduce drift or spray drift, by means including but not limited to increasing the droplet size of a sprayed liquid selected from the group consisting of at least one phospholipid, vegetable colloids, non-derivatized guar gum, non-cationic derivatized guar gum, cationic guar gum, polyethylene oxides, poly(vinyl pyrrolidones), polyacrylamides, a non-ionic emulsifier, a cationic emulsifier and an anionic emulsifier.

According to another embodiment of the present invention, the enhancing deposition, “deposition agent” of the agricultural composition assist in keeping it from drifting from the target area as it is being applied sprayed, or from being blown away from the plant or animal once it has been deposited. The deposition agents include modified cellulose (e.g., carboxymethylcellulose), botanicals (e.g., grain flours, ground plant parts), natural earths (talc, vermiculite, diatomaceous earth), natural clays (e.g., attapulgite, bentonite, kaolinite, montmorillonite), or synthetic (e.g., Laponite) or proteins (soy protein, potato protein, soy flour, potato flour, fish meal, bone meal, yeast extract, blood meal)

According to another embodiment of the present invention, herbicide safeners reduce or eliminate the phytotoxic effects of the active ingredient against non-target plant species (e.g. crops), while maintaining acceptable levels of efficacy against target species (e.g. weeds). Examples of herbicide safeners include, but are not limited to benoxacor, (RS)-4-dichloroacetyl-3,4-dihydro-3-methyl-2H-1,4-benzoxazine; cloquintocet, (5-chloroquinolin-8-yloxy)acetic acid; cloquintocet-mexyl, (RS)-1-methylhexyl (5-chloroquinolin-8-yloxy)acetate; cyometrinil, (Z)-cyanomethoxyimino(phenyl)acetonitrile; cyprosulfamide, N-[4-(cyclopropylcarbamoyl)phenylsulfonyl]-o-anisamide or N-[4-(cyclopropylcarbamoyl)phenylsulfonyl]-2-methoxybenzamide; dichlormid, N,N-diallyl-2,2-dichloroacetamide; dicyclonon, (RS)-1-dichloroacetyl-3,3,8a-trimethylperhydropyrrolo[1,2-a]pyrimidin-6-one; dietholate, 0,0-diethyl O-phenyl phosphorothioate; fenchlorazole, 1-(2,4-dichlorophenyl)-5-trichloromethyl-1H-1,2,4-triazole-3-carboxylic acid; fenchlorazole-ethyl, ethyl 1-(2,4-dichlorophenyl)-5-trichloromethyl-1H-1,2,4-triazole-3-carboxylate; fenclorim, 1-(2,4-dichlorophenyl)-5-trichloromethyl-1H-1,2,4-triazole-3-carboxylic acid; flurazole, benzyl 2-chloro-4-trifluoromethyl-1,3-thiazole-5-carboxylate or benzyl 2-chloro-4-trifluoromethylthiazole-5-carboxylate; fluxofenim, 4′-chloro-2,2,2-trifluoroacetophenone (EZ)-O-1,3-dioxolan-2-ylmethyloxime; furilazole, (RS)-3-dichloroacetyl-5-(2-furanyl)-2,2-dimethyl-1,3-oxazolidine; isoxadifen, 4,5-dihydro-5,5-diphenyl-1,2-oxazole-3-carboxylic acid; isoxadifen-ethyl, ethyl 4,5-dihydro-5,5-diphenyl-1,2-oxazole-3-carboxylate; jiecaowan, 2-(dichloromethyl)-2-methyl-1,3-dioxolane; jiecaoxi, N-allyl-N-(allylcarbamoylmethyl)-2,2-dichloroacetamide; mephenate, 4-chlorophenyl methylcarbamate; naphthalic anhydride, naphthalene-1,8-dicarboxylic anhydride; oxabetrinil, (Z)-1,3-dioxolan-2-ylmethoxyimino(phenyl)acetonitrile; mefenpyr, (RS)-1-(2,4-dichlorophenyl)-5-methyl-2-pyrazoline-3,5-dicarboxylic acid; mefenpyr-diethyl, diethyl (RS)-1-(2,4-dichlorophenyl)-5-methyl-2-pyrazoline-3,5-dicarboxylate, MG-191, 2-(dichloromethyl)-2-methyl-1,3-dioxolane and combinations thereof;

According to another embodiment of the present application, suitable suspension agents selected from the group consisting of attapulgite clay, fumed silica and combinations thereof.

According to another embodiment of the present application, the adhesives used in agricultural composition can be, a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arables, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

According to another embodiment of the present application, the gelling agents can be selected from gelling clay or a polysaccharide gum, bentonite clays, xanthan gum, guar gum, locust bean gum, algal oligosaccharides, and combinations thereof,

According to another embodiment of the present application, suitable examples of water-absorbent agents includes a crosslinked polyacrylic acid polymer; a hydrolyzed starch-acrylonitrile graft polymer; a neutralized starch-acrylic acid graft polymer; a saponified vinyl acetate-acrylic ester copolymer; a crosslinked carboxymethyl cellulose; a hydrolyzed or crosslinked acrylnitryl copolymer or acrylamide copolymer; a crosslinked cationic monomer; a crosslinked isobutylene-maleic acid copolymer; a crosslinked polymer of 2-acrylamide-2-methylpropanesulfonic acid and acrylic acid; and the like.

According to another embodiment of the present application, the water softener agents can be selected from sodium tripolyphosphate and tetra potassium pyrophosphate.

According to another embodiment of the present application, it is contemplated to employ at least one solvent used in the agricultural composition exhibits desirable solvency relative to agricultural compositions. In addition, the solvents can include alkyl benzenes, alkyl naphthalenes such as isophorone, cyclohexanone, and methylcyclohexanone, methyl esters of fatty acids, aliphatic hydrocarbons, cycloaliphatic hydrocarbons, fuel oils, isophorone, methyl ethyl ketone, N-methylpyrolidone, butyl lactate, dimethyl sulfoxide, acetophenone, or petroleum-derived toluene, xylene, chlorotoluene, benzene, methyl isobutyl ketone, cyclohexanone, naphthalene, ketones such as, chlorinated hydrocarbons such as chlorobenzene and trichloroethane, alcohols such as benzyl alcohol, furfuryl alcohol, butanol and glycol ethers and combination thereof.

According to another embodiment of the present application, solid and liquid carriers can be utilized in present application, used to coat seeds with modified maleated soybean compositions or to distribute oil compositions into a field. Solid carriers such as diatomaceous earth, finely ground limestone or magnesium carbonate or calcium carbonate. Sugars such as sucrose, maltose, maltodextrin, or dextrose may also be used as solid carriers. Carriers can further include gases or vapors, such as steam, air, or inert gases such as diatomic nitrogen, which can be used to fluidize a solid composition. Other examples of suitable carrier include talc, bentonite, clay, kaoline, white carbon, vermiculite, slaked lime, silica sand, ammonium sulfate, urea, etc. and liquid carriers such as methylnaphthalene, isopropyl alcohol, xylene, cyclohexanone, water, methanol, ethanol, acetone, dimethylformaldehyde and ethyleneglycol.

According to an embodiment the “filler” can be selected from a group comprising bentonite, sub-bentonite, attapulgite, kaolinites, montmorillonite, bauxite, hydrated aluminas, calcined aluminas, diatomaceous earth, chalk, fuller's earth, dolomite, kiesulguhr, loess, prophyllites, talc, vermiculites, limestone, natural and synthetic silicates, silica and china clay.

According to an embodiment the “surfactant” is meant herein a compound that lowers the surface tension of a liquid, allowing easier spreading. Suitable auxiliary surfactants include, but are not limited to nonionic, cationic, amphoteric, polymeric surfactants.

The nonionic surfactants include, alkoxylates, mainly ethoxylates, and nonionic surfactants, in particular fatty alcohol polyoxyethylene esters, for example lauryl alcohol polyoxyethylene ether acetate, alkyl polyoxyethylene ethers and alkyl polyoxypropylene ethers, for example of linear fatty alcohols, alkylaryl alcohol polyoxyethylene ethers, for example octylphenol polyoxyethylene ether, alkoxylated animal and/or vegetable fats and/or oils, for example corn oil ethoxylates, castor oil ethoxylates, tallow fat ethoxylates, glycerol esters such as, for example, glycerol monostearate, fatty alcohol alkoxylates and oxo alcohol alkoxylates and oleyl alcohol polyoxyethylene ether, alkylphenol alkoxylates such as, for example, ethoxylated isooctylphenol, octylphenol or nonylphenol, tributylphenyl polyoxyethylene ethers, fatty amine alkoxylates, fatty acid amide alkoxylates and fatty acid diethanolamide alkoxylates, in particular their ethoxylates, sugar surfactants, sorbitol esters such as, for example, sorbitan fatty acid esters (sorbitan monooleate, sorbitan tristearate), polyoxyethylene sorbitan fatty acid esters, alkylpolyglycosides, N-alkylgluconamides, -alkylmethyl sulfoxides, alkyldimethylphosphine oxides such as, for example, tetradecyldimethylphosphine oxide.

The amphoteric surfactants include, for example, sulfobetaines, carboxybetaines and alkyldimethylamine oxides, for example tetradecyldimethylamine oxide.

The polymeric surfactants include, for example, di-, tri- and multi-block polymers of the type (AB), ABA and BAB, for example optionally end-capped ethylene oxide/propylene oxide block copolymers, for example ethylenediamine-EO/PO block copolymers, polystyrene block polyethylene oxide, and AB comb polymers, for example polymethacrylate comb polyethylene oxide.

Further surfactants are perfluoro surfactants, silicone surfactants, for example polyether-modified siloxanes, phospholipids such as, for example lecithin or chemically modified lecithins, amino acid surfactants, for example N-lauroylglutamate, and surface-active homo- and copolymers, for example polyvinylpyrrolidone, polyacrylic acids in the form of their salts, polyvinyl alcohol, polypropylene oxide, polyethylene oxide, maleic anhydride/isobutene copolymers and vinylpyrrolidone/vinyl acetate copolymers. Unless specified, the alkyl chains of the abovementioned surfactants are linear or branched radicals, usually having 8 to 25 carbon atoms.

The term “dispersing agent” as used herein refers to a “substance added to a suspension to improve the separation of particles and to prevent settling or clumping. According to an embodiment the dispersing agents which can be used in the agricultural composition can be chosen from a group comprising polyvinylpyrrolidone, polyvinylalcohol, lignosulphonates, phenyl naphthalene sulphonates, ethoxylated alkyl phenols, ethoxylated fatty acids, alkoxylated linear alcohols, polyaromatic sulfonates, sodium alkyl aryl sulfonates, glyceryl esters, maleic anhydride copolymers, phosphate esters, condensation products of aryl sulphonic acids and formaldehyde, condensation products of alkylaryl sulphonic acids and formaldehyde, addition products of ethylene oxide and fatty acid esters, salts of addition products. of ethylene oxide and fatty acid esters, sulfonates of condensed naphthalene, addition products of ethylene oxide and fatty acid esters, salts of addition products of ethylene oxide and fatty acid esters, lignin derivatives, naphthalene formaldehyde condensates, sodium salt of isodecylsulfosuccinic acid half ester, polycarboxylates, sodium alkylbenzenesulfonates, sodium salts of sulfonated naphthalene, ammonium salts of sulfonated naphthalene, salts of polyacrylic acids, salts of phenolsulfonic acids and salts of naphthalene sulfonic acids.

The term “emulsifier” as used herein refers to a substance that stabilizes an emulsion, i.e. a mixture of two or more liquids. The emulsifier can be any single emulsifier or combination of two or more emulsifiers that can produce an agricultural composition. The emulsifier can be anionic, cationic, or non-ionic, or can include a combination of anionic, cationic, and non-ionic emulsifiers.

Examples of non-ionic emulsifiers include alkoxylated castor oil, alkoxylated polyarylphenols, alkoxylated alkyl phenols, alkoxylated alkylarylphenols, and alkoxylated fatty alcohols, as well as mixtures thereof. More specific examples include alkoxylated acetylenic diols, polyoxyalkylene mono- and di(alkyl)phenylethers, polyoxyalkylene di- and tristyrylphenylethers, block copolymers of ethylene oxide and propylene oxide and C2-C6 alkyl adducts thereof, sorbitan C8-C20 mono-, di- and tri(C8-C20 fatty acid) esters, polyoxyalkylene sorbitan mono-, di- and tri(C8-C20 fatty acid) esters, sucrose esters and C8-C20 alkyl polyglycosides.

Commercially available examples of non-ionic emulsifiers include the following: those commercially available under the trade names POLYSORBATE 80 (aka, Tween 80; Polyoxyethylene (20) sorbitan monooleate) (HLB=15); Tween 20 (Polyoxyethylene sorbitan monolaurate), Tween 85 (aka emulsifier T-85) (HLB=11.0); TRITON X-100 (aka octylphenol ethoxylate) (HLB=13.4).

Examples of useful anionic emulsifiers include calcium-dodecylbenzene sulfonate, dioctyl sulfosuccinate, sulfated or phosphated alkoxylated fatty alcohols, sulfated or phosphated alkoxylated alkylaryl phenols, and combinations of these. Some more specific examples include alkyl carboxylates including fatty acids, alcohol sulfates, alcohol phosphate mono- and diesters, (alkyl)phenol polyoxyethylene ether carboxylates, sulfates and sulfonates, (alkyl)phenol polyoxyethylene phosphate mono- and diesters, alkylbenzene sulfonates, naphthalene sulfonates and formaldehyde condensates thereof, lignosulfonates, alkyl sulfosuccinates and sulfosuccinamates, alkyl polyoxyethylene sulfosuccinates and sulfosuccinamates, and C8-20 acyl glutamates, sarcosinates, isethionates and taurates. Cationic counterions accompanying an anionic emulsifier can preferably be monovalent, e.g., hydrogen, sodium, potassium, ammonium, and monovalent organic ammonium cations (isopropylamine).

Examples of emulsifiers which exhibit cationic properties in acidic conditions are fatty amines, amine oxides and amine ethoxylates. Amphoteric emulsifiers such as betaines may also exhibit such properties. Preferably, the cationic emulsifiers are selected from dimethylcocoamine, dimethyl-laurylamine oxide, alkyltrimethylammonium chloride, alkyl dimethylbenzylammonium chloride, alkylpyridium chloride, alkylimidazolium chloride, or mixtures thereof.

Preferably, the nonionic emulsifiers are alkyl polysaccharides, sorbate emulsifiers, alkyl bearing ethoxylates or fatty alkanolamides. Alkyl polysaccharides are sometimes called alkyl polyglucosides, alkyl glucosides or alkyl saccharides. The sorbate emulsifiers are sorbitan mono-(or sesqui-) esters of fatty acids and include sorbitan mono-oleate and sorbitan monolaurate.

According to an embodiment the wetting agent which can be used in the agricultural composition can be selected from a group comprising carbinol (hydroxyl) terminated polydimethylsiloxane (CAS 67674-67-3), Silwet 408 type wetting agents, phenyl naphthalene sulphonates, alkyl naphthalene sulfonate, sodium alkyl naphthalene sulfonate, sodium salt of sulfonated alkylcarboxylate, polyoxyalkylated ethyl phenols, polyoxyethoylated fatty alcohols, polyoxythoxylated fatty amines, lignin derivatives, alkane sulfonates, alkylbenzene sulfonates, salts of polycarboxylic acids, salts of esters of sulfosuccinic acid, alkylnaphthalenesulphonates, alkylbenzenesulfonates, alkylpolyglycol ether sulfonates, alkyl ether phosphates, alkyl ether sulphates and alkyl sulfosuccinic monoesters. Examples of a wetting agent also include polyoxyethylenealkyl phenyl ethers, sodium alkylbenzenesulfonates, dioctyl sulfosuccinate, sodium alkylnaphthalenesulfonates, sodium alkylsulfates, sodium alkylsulfosuccinates and polyoxyethylenealkyl aryl ether or Break-thru DA 675 an aqueous solution of an organic modified polymer with pigment binding groups, which can be used alone or in combination of two or more.

The agriculture compositions can include a solubilizer to ensure good solubilization and/or dissolution of the active ingredients such as the fungicide and/or lipopeptide. A solubilizer can also be added to increase the solubility of the fungicide and/or lipopeptide and/or other components, such as surfactants, or to maintain the composition as a stable or homogeneous solution or dispersion.

Examples of suitable solubilizers of agricultural composition include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, F-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof; and other solubilizers known in the art, such as dimethyl acetamide, dimethyl isosorbide, N-methylpyrrolidones, monooctanoin, diethylene glycol monoethyl ether, and water.

A solubilizer for use in the present invention can be any substance that increases the solubility of an agricultural composition in water. Preferred solubilizers include benzyl acetate; N-methyl pyrrolidone; propylene carbonate; alcohols, such as benzyl alcohol, ethanol, methanol; and mixtures thereof. In some embodiments, solubilizers include sorbitol, triacetin, ethyl alcohol, PEG-400, glycofurol and propylene glycol.

The term “penetration enhancer” used herein as a compound that accelerates the uptake of active ingredient through the cuticle of a plant into the plant, i.e., the rate of uptake, and/or increases the amount of active ingredient absorbed into the plant. Classes of substances known as penetration enhancers, include alkyl phosphates, such as tributyl phosphate and tripropyl phosphate, and naphthalenesulphonic acid salts.

Suitable stickers/adhesion agents include block copolymer EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers and copolymers derived from these polymers.

Thickener is a kind of auxiliary rheological agents; Rheological characteristic to the hierarchy of control plays important effect, and it can improve system viscosity, makes system keep stable suspended state or milkiness state uniformly; or the formation gel, can give the physical and chemical stability ability of product fine. The thickener kind is a lot, can be divided into water-based thickener, oiliness thickener, acid thickener, alkaline thickener through its purposes. When selecting these thickeners except flowability, transparency, denseness, gelation and the suspended particulate ability that will consider product. Thickners/stabilizers such as natural polymers such as Carboxymethyl cellulose (CMC), guar gum, locust bean gum, xanthan gum, natural gums, hydroxyethylcellulose (HEC), HPMC or synthetic polymers based on acrylates, polyacrylamide (PAM), PVP or combination of synthetic polymers and carbomers.

Examples of the thickener include organic thickeners such as xanthan gum, polyvinyl pyrrolidone, polyvinyl alcohol, polyethylene glycol, methylcellulose, and alginic acid, and inorganic thickeners such as bentonite. If desired, a thickening agent can also be added, such as for example a thickener known as Aqualon. Aqualon is a trademark of Hercules Inc., of Wilmington, Delaware, United States of America. Aqualon is a cellulose ether adhesive thickener. Other thickeners include hydroxy ethyl cellulose, carboxy methyl propyl cellulose, and the like.

According to another embodiment of the present invention, the humectant will act to absorb moisture during humid periods of time and the moisture so held by the humectant will then be extracted by the powerful osmosis action of root fibres during dry hot periods of time (such as during dry hot days). Humectants which have surprisingly been found to have substantial advantages in improving plant root watering, include sorbitol, glycerol, molasses, potassium lactate, sodium lactate and potassium acetate.

According to another embodiment of the present invention, the repellent substances are known to cause insects to be driven away from or to reject (otherwise insect-acceptable) food sources. Repellents may be in the form of gases (olfactory), liquids, or solids (gustatory). Insect repellents include, benzil; benzyl benzoate; 2,3,4,5-bis(butyl-2-ene) tetrahydrofurfural (MGK Repellent 11); butoxypolypropylene glycol; N-butylacetanilide; normal-butyl-6,6-dimethyl-5,6-dihydro-1,4-pyrone-2-carboxylate (Indalone); dibutyl adipate; dibutyl phthalate; di-normal-butyl succinate (Tabatrex); N,N-diethyl-meta-toluamide (also known as Delphone, Detamide, Autan, or, more simply, Deet); dimethyl carbate (cis-bicyclo-[2.2.1]-5-heptene-2,3-dicarboxylate); dimethyl phthalate; 2-ethyl-2-butyl-1,3-propanediol; 2-ethyl-1,3-hexanediol (Rutgers 612); di-normal-propyl isocinchomeronate (MGK Repellent 326); 2-phenylcyclohexanol; and normal-propyl N, N-diethylsuccinamate. Standard repellents for mosquitoes, ticks, and the like are citronella oil, dimethyl phthalate, normal-butylmesityl oxide oxalate and 2-ethyl hexanediol-1,3. A particular group of insect repellents includes the terpenoid compounds, Terpenoid-alcohol or terpene-ols are terpenoids which have at least one hydroxyl group. Examples of terpene-ols include: perillyl alcohol, carveol, myrtenol, and cis-verbenol; myrtanol, iso-pinocampheol, dihydrocarveol, isopulegol, terpineol, terpinen-4-ol, nerol, geraniol, and linalool, menthol, beta-citronellol, and dihydro-myrcenol.

The agricultural composition further comprises feeding stimulants or attractants include floral odor, light, pheromone, a plant extract, a sugar, honey, molasses, a protein, or combinations of amino acids in varying amounts; or other attractants adaptable to multiple insect species to attract insects to consume or otherwise be exposed to the active ingredients. The food substance includes a plant extract, a sugar, honey, molasses, a protein, or combinations of amino acids in varying amounts; or other fluid or material adaptable to be ingested by multiple insect species.

The agricultural compositions can include a feeding stimulant (also known as a gustatory stimulant) for the insect to be controlled, a 1-arylpyrazole and/or nicotinyl insecticide. Suitable feeding stimulants include: proteins, including animal proteins and plant proteins, e.g. in the form meat meal, fish meal, fish extracts, seafood, seafood extracts, or blood meal, insect parts, crickets powder, yeast extracts, egg yolk, protein hydrolysates, yeast autolysates, gluten hydrolysates, and the like; carbohydrates and hydrogenated carbohydrates, in particular mono- and disaccharides such glucose, arabinose, fructose, mannose, sucrose, lactose, galactose, maltose, maltotriose, maltotetraose, maltopentaose or mixtures thereof such as molasses, corn syrup, maple syrup, invert sugars, and honey; polysaccharides including starch such as potato starch, corn starch, and starch based materials such as cereal powders (e.g. wheat powder, maize powder, malt powder, rice powder, rice bran), pectines, and glycerol, hydrogenated mono- and oligosaccharides (sugar alcohols) such as xylitol, sorbitol, mannitol, isomaltolose, trehalose and maltitol as well as maltitol containing syrups; fats and oils, such as vegetable oils, e.g. corn oil, olive oil, caraway oil, linseed oil, canola oil, peanut oil, rape seed oil, sesame oil, soy bean oil, sunflower oil, fats and oils of animal origin such as fish based oil, and also fatty acids derived from the aforementioned fats and oils

Suitable compatibilizer can be either a non-reactive compatibilizer or a reactive compatibilizer. Specific examples of reactive compatibilizer include, but are not limited to: long chain fatty acids, such as stearic acid (octadecanoic acid); long chain fatty acid chlorides, such as stearoyl chloride (octadecanoyl chloride); long chain fatty acid anhydrides, such as stearic anhydride (octadecanoic anhydride); epoxidized oils and fatty esters; styrene maleic anhydride copolymers; maleic anhydride grafted polypropylene; copolymers of maleic anhydride with olefins and/or acrylic esters, such as terpolymers of ethylene, acrylic ester and maleic anhydride; and copolymers of glycidyl methacrylate with olefins and/or acrylic esters, such as terpolymers of ethylene, acrylic ester, and glycidyl methacrylate.

Examples of non-reactive compatibilizers include, but are not limited to, ethoxylated alcohols, ethoxylated alkylphenols, ethoxylated fatty acids, block polymers of propylene oxide and ethylene oxide, polyglycerol esters, polysaccharide esters, and sorbitan esters. Examples of ethoxylated alcohols are C11-C15 secondary alcohol ethoxylates, polyoxyethylene cetyl ether, polyoxyethylene stearyl ether, and C12-014 natural liner alcohol ethoxylated with ethylene oxide. C11-C15 secondary ethyoxylates can be obtained as Dow Tergitol® 15S from the Dow Chemical Company. Polyoxyethlene cetyl ether and polyoxyethylene stearyl ether can be obtained from ICI Surfactants under the Brij® series of products. C12-C14 natural linear alcohol ethoxylated with ethylene oxide can be obtained from Hoechst Celanese under the Genapol® series of products. Examples of ethoxylated alkylphenols include octylphenoxy poly(ethyleneoxy)ethanol and nonylphenoxy poly(ethyleneoxy)ethanol. Octylphenoxy poly(ethyleneoxy)ethanol can be obtained as Igepal® CA series of products from Rhodia, and nonylphenoxy poly(ethyleneoxy)ethanol can be obtained as Igepal CO series of products from Rhodia or as Tergitol® NP from Dow Chemical Company. Ethyoxylated fatty acids include polyethyleneglycol monostearate or monolaruate which can be obtained from Henkel under the Nopalcol® series of products. Block polymers of propylene oxide and ethylene oxide can be obtained under the Pluronic® series of products from BASF. Polyglycerol esters can be obtained from Stepan under the Drewpol® series of products. Polysaccharide esters can be obtained from Henkel under the Glucopon® series of products, which are alkyl polyglucosides. Sorbitan esters can be obtained from ICI under the Tween® series of products.

The fungicides or bactericides include organic sulfur fungicides or bactericides such as zineb agent, maneb agent, thiram agent, mancozeb agent, polycarbamate agent, propineb agent etc., benzimidazole fungicides or bactericides such as benomyl agent, thiophanate-methyl agent etc., dicarboxylic acid fungicides or bactericides such as iprodione agent, procymidone agent etc., other synthetic fungicides or bactericides such as triazine agent, iminoctadine triacetate agent, isoprothiolane agent, TPN agent, probenazole agent, captan agent, fluoromide agent, DPC agent, and iminoctadine albesilate agent, sterol biosynthesis inhibitors such as triflumizole agent, bitertanol agent, pyrifenox agent, fenarimol agent, triforine agent, triadimefon agent, myclobutanil agent, difenoconazole agent, and imibenconazole agent, acid amide fungicides or bactericides such as metalaxyl agent, and mepronil agent, copper fungicides or bactericides such as inorganic copper agent, and organic copper agent, antibiotic fungicides or bactericides such as streptomycin agent, polyoxin agent, blasticidin S agent, kasugamycin agent, validamycin agent, and oxytetracycline agent, soil fungicides or bactericides such as etridiazole agent, and hymexazol agent, melamine biosynthesis inhibitors such as phthalide agent, carpropamid agent, organic phosphorus fungicides or bactericides such as IBP agent, EDDP agent, and fosetyl agent, inorganic insecticides such as inorganic sulfur agent, and hydrogen carbonate agent, methoxyacrylate fungicides or bactericides such as azoxystrobin, and kresoxim-methyl agent, anilinopyrimidine fungicides or bactericides such as mepanipyrim agent, synthetic antibiotics such as oxolinic acid agent, naturally occurring fungicides or bactericides such as soybean lecithin, and bactericides derived from living things such as antagonistic bactericides.

In at least one embodiment, the agricultural compositions described herein can comprise one or more anti-freezing agents. Non-limiting examples of anti-freezing agents include ethylene glycol, propylene glycol, urea, glycerin, and combinations thereof.

Suitable examples of solvent crystallization inhibitors include, but are not limited to, NMP, DMA, DMSO, or PEG. Examples of crystallization preventive agents include ethylene glycol, propylene glycol, glycerin, cyclodextrins, alkylene oxide adduct of glycerin, and the like.

Defoamers and antifoaming agents are used to reduce the foaming while spraying. These include various defoamers based on silicone defoamers, organo-modified siloxanes, kerosene defoamers, wax defoamers, oil-based deformers such as turkey red oil and fatty acid based defoamers.

Suitable examples of anti-foaming agents include, but are not limited to, silicone based anti-foaming agents (e.g., aqueous emulsions of dimethyl polysiloxane, FG-10 from Dow-Corning®, Trans 10A from Trans-Chemo Inc.), and non-silicone based anti-foaming agents such as octanol, nonanol, and silica. Examples of the antifoaming agent can include silicone emulsion, silicone oil, acetylene diol, and PO/EO block polymer.

Suitable examples of colorants or dyes and brighteners include one or more azoic dye, azo dye, acridine, aniline dye, aniline black, indanthrene, eosin, congo red, dihydroindole, methylene blue, phenazine derivative dye, neutral red, phenolphthalein, fuchsin, fluorescein, para red, mauve, carotene; carotenoids such as xanthophylls, cryptoxanthin, zeaxanthin, fucoxanthin, lycopene, lutein; flavonoids such as flavones, flavanones, ants crawl, anthocyans, catechin; quinones such as melanin; porphyrin dye such as chlorophyll, chlorophyllide, bacteriochlorophyll, cytochrome, pheophorbide, Feo porphyrin, hemerythrin, hemoglobin, hemovanadin, hemocyanin, porphyrin, porphine, myoglobin; phycobilin-based pigments such as phycocyanin, phycobilin, phycoerythrin, phytochrome, biliverdin, bilirubin; alizarin, anthocyans, anthraquinone, indigo, urobilin, erythrocruorin, carthamin, hexane cretin Chin, curcumin, crocetin, chlorine, chlorocruorin, genistein, cochineal, Gosshiporu, commelinin, shikonin, Suterukopirin, tannin, Tsurashin, bixin, hypericin, Pin'nagurobin, brazilin, purpurin, betacyanin, berberine, Horubirin, mangosteen (mangostin), Morinjin, laminaran, leghemoglobin, litmus, rhodopsin, rhodoxanthin, Rodomachin, carbon black, and red iron oxide.

Suitable examples of the various classes of tackifiers include, but are not limited to, aliphatic resins, polyethylene resins, hydrogenated resins, mixed aliphatic-aromatic resins, styrene/α-methylene styrene resins, pure monomer hydrocarbon resin, hydrogenated pure monomer hydrocarbon resin, modified styrene copolymers.

Suitable examples of suitable binders includes gum arabic, glue, gelatin, casein, starch, cellulose esters, aliphatic polyester, polyalkanoate, aliphatic-aromatic polyesters, sulfonated aliphatic-aromatic polyester, polyesteramide, rosin/polycaprolactone triblock copolymer, rosin/polyadipate second diester triblock copolymer, rosin/poly-butanedioic acid second diester triblock copolymer, polyvinyl acetate, EVA ester, polyethylene co-acrylic acid ethyl ester, polyethylene co-acrylic acid methyl esters, polyethylene-co-propylene, polyethylene-co-1-butylene, polyethylene-co-1-amylenes, polystyrene, acrylic compounds, polyurethane, urethane-styrene polymer dispersion, nonionic polyesters urethane dispersion, acrylic acid dispersion, silanization (silanated) anionic acrylic ester-styrene polymer dispersion, anionic acrylic ester-styrene-acrylonitrile dispersion, Acrylic ester-acrylonitrile dispersion, vinyl pyrrolidone/styrene copolymer latexes are (for example 430, produced by ISP chemical companies), the vinylacetate-ethylene dispersion of carboxylated and non-carboxylated, vinyl acetate homopolymer dispersion, polyvinyl chloride emulsion, polyvinylidene fluoride dispersion, ethylene acrylic dispersion, polyamide dispersion, anionic carboxyl or non-carboxylated acrylonitrile nitrile-butadiene-styrene emulsion and acrylonitrile emulsion, by styrene derived resin dispersion, as aliphatic series and/or aromatic hydrocarbon derived from resin dispersion, styrene maleic anhydride and their mixtures thereof.

According to an embodiment the binding agent which can be used in the agricultural composition can be selected from a group comprising polyvinyl alcohols, phenyl naphthalene sulphonates, lignin derivatives, polyvinyl pyrrolidone, polyalkylpyrrolidone, carboxymethylcellulose, xanthan gum, polyethoxylated fatty acids, polyethoxylated fatty alcohols, ethylene oxide copolymer, propylene oxide copolymer, polyethylene glycols and polyethylene oxides.

According to an embodiment the preservative includes but not limited to, aqueous dipropylene glycol solution of 1,2-benzisothiazolin-3-one, propionic acid and salt thereof, salicylic acid and salt thereof, 2; 4-hexadienoic acid (sorbic acid) and salt thereof, formaldehyde and paraformaldehyde, 2-xenol ether and salt thereof, 2-zinc sulfenyl pyridine N-oxides, benzoic acid, its ester and salt, P-hydroxybenzoic acid (p-hydroxybenzoate), its ester and salt.

The pH adjuster used in this application is effective for providing an alkaline pH of above about 7 up to about 10 for the final spray solution that is applied to the plants. The pH adjuster may be organic and/or inorganic. Examples of pH adjusters include triethanolamine, primary amino alcohols, ammonium hydroxide and mixtures thereof.

The example of suitable stabilizers includes but not limited to, agar, alginic acid, alginate, calcium lactobionate, carrageenan, gellan gum and guar gum. Composition of the present invention also can comprise two kinds or more how different stabilizing agents.

In some embodiments, the suitable range of the agricultural composition having a reaction product of a polymer is having a weight-average molecular weight from about 3,000 Daltons to about 20,000 Daltons and a weight-average degree of polymerization, n, of about 3 to about 20.

In some embodiments, the suitable range of the agricultural composition having a reaction product of a polymer is having a viscosity measured at 25° C. with a Brookfield viscometer ranging from about 100 to 10,000 cps.

However, those skilled in the art that it is possible to utilize other agricultural ingredients known in the art without departing from the scope of the claims of the present invention.

In some embodiments, the suitable range of the agricultural composition comprising (A) for the present application can be varied from about 0.01 wt. % to about 1.0 wt. %; from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. % based on the total weight of the agricultural composition.

In some embodiments, the suitable range of the agricultural active ingredient (B) for the present application can be varied from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; .%; or from about 70 wt. % to about 75 wt. %; or from about 75 wt. % to about 80 wt. %; or from about 80 wt. % to about 85 wt. %; or from about 85 wt. % to about 90 wt. % based on the total weight of the agricultural composition.

In some embodiments, the suitable range of additional ingredient (C) for the present application can be varied from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. %; or from about 90 wt. % to about 95 wt. %; or from about 95 wt. % to about 99.9 wt. % based on the total weight of the agricultural composition.

In one non-limiting embodiment, the present invention provides an agricultural composition in the form of an aqueous or a non-aqueous composition comprising a capsule suspension, an emulsifiable concentrate, an emulsion for seed treatment, a concentrated aqueous emulsion, a microemulsion, a suspoemulsion, an oil-in-water emulsion, a flowable concentrate for seed treatment, an oil dispersion, a suspension concentrate, a water dispersible granule or a wettable powder.

In one non-limiting embodiment, the present invention provides a method for treating a vegetation comprising applying an agricultural composition to a plant, plant foliage, blossoms, leaves, stems, fruits, the area adjacent to the plant, soil, seeds, germinating seeds, roots, liquid and solid growth media, flower, bud, and hydroponic growth solutions.

In one non-limiting embodiment, the present invention provides a method of treating an animal comprising topically applying an agricultural composition to an area of skin of an animal.

The reactions and compositions according to the application may be analyzed by known techniques. Especially preferred are the techniques of 13C nuclear magnetic resonance (NMR) spectroscopy, gas chromatography (GC), Infra-red (IR), Liquid Chromatography (LC) and gel permeation chromatography (GPC) in order to decipher identity, residual monomer concentrations, molecular weight, and molecular weight distribution.

Further, certain aspects of the present application are illustrated in detail by way of the following examples. The examples are given herein for illustration of the application and are not intended to be limiting thereof.

### EXAMPLES

### Example A: Grafting of Maleic Anhydride onto Natural Oils

### Example A1: Grafting of Maleic Anhydride onto Soybean Oil

A 1-L, 4-neck kettle equipped with a thermocouple, a condenser, a nitrogen sparge adapter, and a mechanical stirrer, was charged with 600 g of soybean oil and 204 g (3 mole equivalents, relative to soybean oil) of maleic anhydride. The mixture was sparged with nitrogen for 15 minutes at room temperature. The mixture was heated slowly from room temperature to 210° C. and was held at 210° C. for 6-8 hours. Completion of the reaction was indicated by NMR and <0.05% residual maleic anhydride by LC.

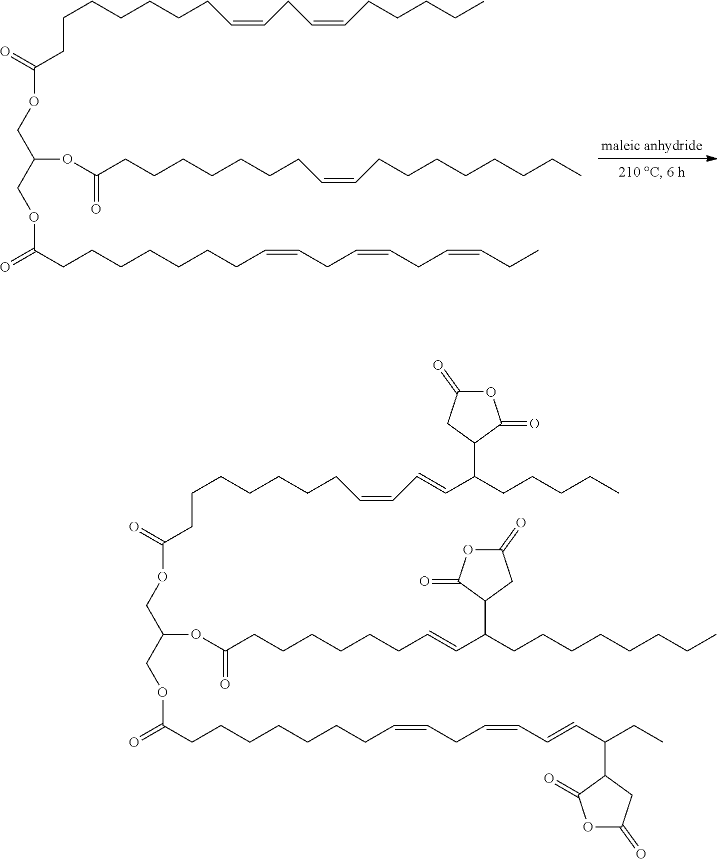 [SMILES: CC/C=C\C/C=C\C/C=C\CCCCCCCC(=O)OCC(COC(=O)CCCCCCC/C=C\C/C=C\CCCCC)OC(=O)CCCCCCC/C=C\CCCCCCCC ; CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C1CC(=O)OC1=O)C1CC(=O)OC1=O]

### Example A2: Grafting of Maleic Anhydride onto Palm Oil

In a 1-L, 4-neck kettle equipped with a thermocouple, a condenser, a nitrogen sparge adapter, and a mechanical stirrer, 100 g of Palm oil and 23 g (2 mole equivalents based on palm oil) of Maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber-colored, viscous product which was characterized by NMR and LC<1% residual maleic anhydride.

### Example A3: Grafting of Maleic Anhydride onto Canola Oil

In a 1-L, 4-neck kettle equipped with a thermocouple, a condenser, a nitrogen sparge adapter, and a mechanical stirrer, 100 g of Canola oil and 22.2 g (2 mole equivalents based on canola oil) of Maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber-colored, viscous product which was characterized by NMR and LC<1% residual maleic anhydride.

### Example A4: Grafting of Maleic Anhydride onto Sunflower Oil

In a 1-L, 4-neck kettle equipped with a thermocouple, a condenser, a nitrogen sparge adapter, and a mechanical stirrer, 100 g of Sunflower oil and 22.4 g (2 mole equivalents based on sunflower oil) of Maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber-colored, viscous product which was characterized by NMR and LC<1% residual maleic anhydride.

### Example A5: Grafting of Maleic Anhydride onto Castor Oil

In a 1-L, 4-neck kettle equipped with a thermocouple, a condenser, a nitrogen sparge adapter, and a mechanical stirrer, 100 g of yellow color Castor oil and 21 g (2 mole equivalents based on castor oil) of Maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber-colored, viscous product which was characterized by NMR and LC<1% residual maleic anhydride.

### Example B: Salts of Maleated Natural Oils

### Example B1: Sodium Salt of Maleated Soybean Oil from Aqueous Sodium Hydroxide

In a 1-L, 4-neck kettle equipped with a thermocouple, a condenser, a nitrogen purge adapter, and a mechanical stirrer, 100 g of amber-colored, viscous product from maleation reaction A1 was heated to 90° C. and added a mixture of 20.20 g (3 mole equivalents) of 50% aqueous sodium hydroxide and 70 g (46 mole equivalents) water over 1 hour controlling foaming and held for 3 hours. The reaction yields >96% flowable amber-colored 70% active water-soluble product which was characterized by IR, NMR and LC<0.05% residual maleic anhydride.

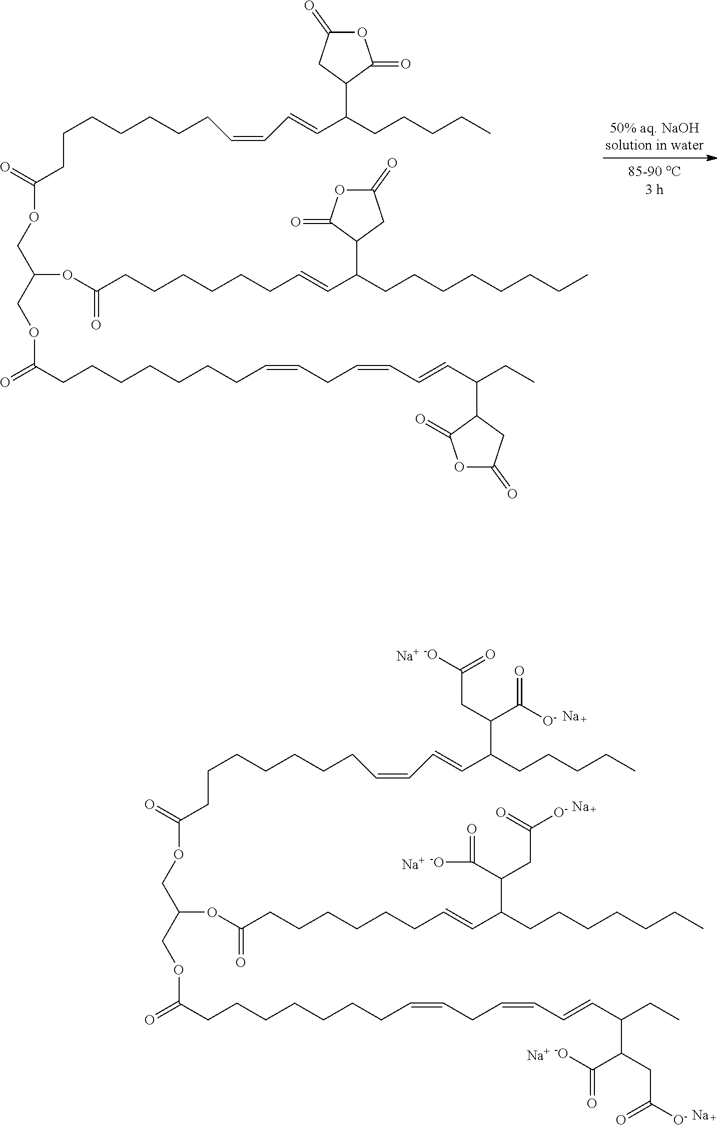

### Example B2: Sodium Salt of Maleated Palm Oil from Aqueous Sodium Hydroxide

In a 1-L, 4-neck kettle equipped with a thermocouple, a condenser, a nitrogen purge adapter, and a mechanical stirrer, 123 g of amber-colored, viscous product from maleation reaction A 2 was heated to 90° C. and add a mixture of 18.04 g (2 mole equivalents) of 50% aqueous sodium hydroxide and 45 g (22 mole equivalents) water over 1 hour controlling foaming and held for 3 hours. The reaction yields >96% flowable amber-colored 70% active water-soluble product which was characterized by IR, NMR and LC<0.05% residual maleic anhydride.

### Example B3: Sodium Salt of Maleated Canola Oil from Aqueous Sodium Hydroxide

In a 1-L, 4-neck kettle equipped with a thermocouple, a condenser, a nitrogen purge adapter, and a mechanical stirrer, 123 g of amber-colored, viscous product from maleation reaction A3 was heated to 90° C. and add a mixture of 18.25 g (2 mole equivalents) of 50% aqueous sodium hydroxide and 45 g (22 mole equivalents) water over 1 hour controlling foaming and held for 3 hours. The reaction yields >96% flowable amber-colored 70% active water-soluble product which was characterized by IR, NMR and LC<0.05% residual maleic anhydride.

### Example B4: Sodium Salt of Maleated Sunflower Oil from Aqueous Sodium Hydroxide

In a 1-L, 4-neck kettle equipped with a thermocouple, a condenser, a nitrogen purge adapter, and a mechanical stirrer, 123 g of amber-colored, viscous product from maleation reaction A4 heated to 90° C. and add a mixture of 18.26 g (2 mole equivalents) of 50% aqueous sodium hydroxide and 45 g (22 mole equivalents) water over 1 hour controlling foaming and was held for 3 hours. The reaction yields >96% flowable amber-colored 70% active water-soluble product which was characterized by IR, NMR and LC<0.05% residual maleic anhydride.

### Example B5: Sodium Salt of Maleated Castor Oil from Aqueous Sodium Hydroxide

In a 1-L, 4-neck kettle equipped with a thermocouple, a condenser, a nitrogen purge adapter, and a mechanical stirrer, 123 g of amber-colored, viscous product from maleation reaction A5 was heated to 90° C. and add a mixture of 17.15 g (2 mole equivalents) of 50% aqueous sodium hydroxide and 45 g (23 mole equivalents) water over 1 hour controlling foaming and was held for 3 hours. The reaction yields >96% flowable amber-colored 70% active water-soluble product which was characterized by IR, NMR and LC<0.05% residual maleic anhydride.

### Example B6: Maleated Soybean Oil, Free Acid

In a 500-mL, 4-neck kettle equipped with a thermocouple, a reflux condenser, a nitrogen purge adapter, and a mechanical stirrer, 100 g of amber-colored, viscous product from maleation reaction A1 was heated to 70° C. Water (140 g) was added, and the mixture was heated to 90° C. and held at 90° C. for 6 hours. IR analysis of the milky suspension confirmed complete hydrolysis of the anhydride groups. Water was removed by distillation to generate the diacid intermediate as a viscous, amber-colored material (>97% solids).

### Example B7: Ammonium Salt of Maleated Soybean Oil

In a 500-mL, 4-neck kettle equipped with a thermocouple, a reflux condenser, a nitrogen purge adapter, and a mechanical stirrer, 60 g of the maleated soybean oil, free acid, produced as in Example B6, was heated to 70° C., and 200 g of water was added. The white dispersion was cooled to 50° C., and 22.4 g of 28-30% aqueous ammonium hydroxide (3.5 mole equivalents) was added over 30 minutes to reach pH 8.6. The product was a homogenous, clear, yellow-colored water-soluble product (20-25% solids), which was characterized by IR, NMR and LC (<0.05% residual maleic anhydride).

### Example B8: 3-Aminopropanol Salt (1.0 Equivalent) of Maleated Soybean Oil

In a 1-L, 4-neck kettle equipped with a thermocouple, reflux condenser, nitrogen purge adapter, and mechanical stirrer, 100 g of maleated soybean oil, produced as in Example A1, and 100 g of water were heated to 90° C. for 6 hours. After hydrolysis was confirmed by IR, the yellow biphasic mixture was cooled to 25° C., and 6.57 g (1.0 mole equivalent, relative to soybean oil) of of 3-amino-1-propanol was added over 30-60 minutes, maintaining the internal temperature below 50° C. The mixture was held for 2 hours at room temperature. The amber, clear to hazy product was characterized by NMR and IR. The pH was 4.5.

### Example B9: 3-Aminopropanol Salt (1.3 Equivalents) of Maleated Soybean Oil

The reaction was run as in Example B8, except that 8.54 g (1.3 mole equivalents, relative to soybean oil) of 3-amino-1-propanol was used. The amber, clear to hazy product was characterized by NMR and IR.

### Example B10: 3-Aminopropanol Salt (2.0 Equivalents) of Maleated Soybean Oil

The reaction was run as in Example B8, except that 13.1 g (2.0 mole equivalents, relative to soybean oil) of 3-amino-1-propanol was used. The amber, clear to hazy product was characterized by NMR and IR.

### Example B11: 3-Aminopropanol Salt (3.0 Equivalents) of Maleated Soybean Oil

The reaction was run as in Example B8, except that 19.7 g (3.0 mole equivalents, relative to soybean oil) of 3-amino-1-propanol was used. The amber, clear to hazy product was characterized by NMR and IR.

### Example B12: 3-Aminopropanol Salt (4.0 Equivalents) of Maleated Soybean Oil

The reaction was run as in Example B8, except that 26.3 g (4.0 mole equivalents, relative to soybean oil) of 3-amino-1-propanol was used. The amber, clear to hazy product was characterized by NMR and IR.

### Example B13: 3-Aminopropanol Salt (5.0 Equivalents) of Maleated Soybean Oil

The reaction was run as in Example B8, except that 32.9 g (5.0 mole equivalents, relative to soybean oil) of 3-amino-1-propanol was used. The amber, clear to hazy product was characterized by NMR and IR. The pH was 8.0.

### Example B14: 3-Aminopropanol Salt of Maleated Soybean Oil, 50% Solids in Ethanol

In a 1-L, 4-neck kettle equipped with a thermocouple, reflux condenser, nitrogen purge adapter, and mechanical stirrer, 200 g of maleated soybean oil, produced as in Example A1, and 200 g of water were heated to 90° C. and held there for 6 hours. Complete hydrolysis of the anhydride groups in the yellow, biphasic mixture was confirmed by NMR and IR. Water was removed by distillation. The thick amber residue was dissolved in 200 g of ethanol at 50-60° C., and heating was stopped. At 50° C., 65.4 g (5 mole equivalents, relative to soybean oil) of 3-amino-1-propanol was added over 1 hour, maintaining the internal temperature below 50° C. The mixture was allowed to cool to room temperature and held for 2 hours at room temperature. The amber, jelly-like product was 50% solids and was characterized by NMR and IR.

### Example B15: 3-Aminopropanol Salt of Maleated Soybean Oil, 30% Solids in Ethanol

The procedure was run as in Example B14, except using 400 g of ethanol to dissolve the amber residue left after removing water by distillation.

### Example C1. Sodium Salt of Maleated Soybean Oil from Sodium Carbonate

In a 1-L, 4-neck kettle equipped with a thermocouple, a condenser, a nitrogen purge adapter, and a mechanical stirrer, 100 g of amber-colored, viscous product from maleation reaction A1 was heated to 90° C., and a mixture of 27.86 g (3 mole equivalents) of sodium carbonate dissolved in 85 g (55 mole equivalents) of water was added over 3 hours, controlling the foam formation. The reaction was held at 90° C. for 3 hours. The reaction yielded >50% flowable, light-yellow colored, 56% active, water-soluble product, which was characterized by IR and LC (<0.05% residual maleic anhydride).

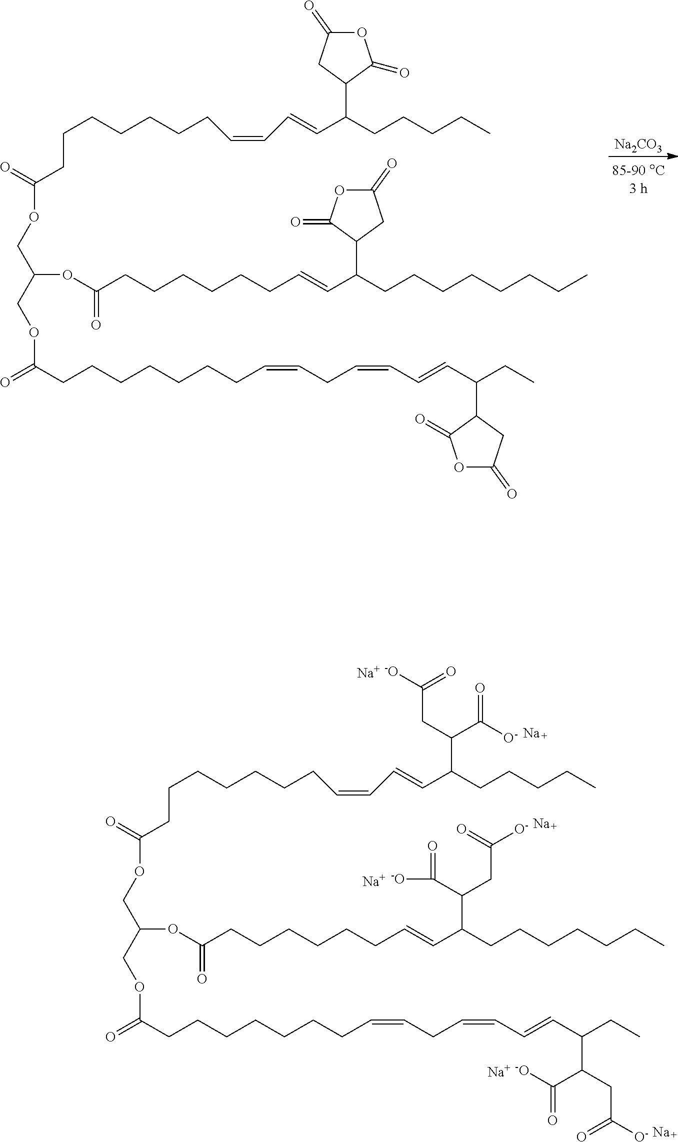

### Example D: Sodium Calcium Salt of Maleated Natural Oil

### Example D1: Sodium Calcium Salt of Maleated Soybean Oil from Calcium Carbonate and Sodium Carbonate (7:3 Ratio)

In a 1-L, 4-neck kettle equipped with a thermocouple, a condenser, a nitrogen purge adapter, and a mechanical stirrer, 100 g of amber-colored, viscous product from maleation reaction A1 was heated to 90° C., and a mixture of 8.36 g (0.9 mole equivalent) of sodium carbonate and 17.8 g (2.1 mole equivalents) of calcium carbonate dissolved in 85 g (55 mole equivalents) of water over 3 hours, controlling the foam formation. The reaction was held at 90° C. for 3 hours. The reaction yielded >90% flowable, light-yellow colored, 60% active, water-soluble product, which was characterized by IR and LC (<0.05% residual maleic anhydride).

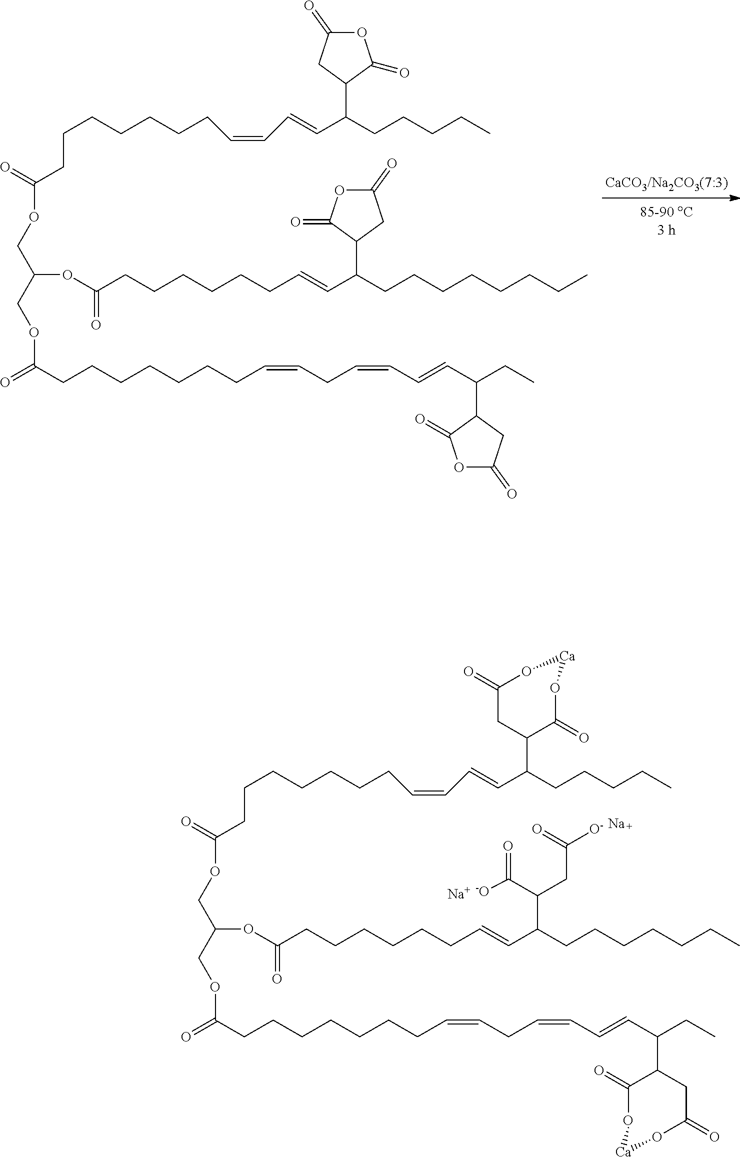

### Example D2: Sodium Calcium Salt of Maleated Palm Oil from Calcium Carbonate and Sodium Carbonate (7:3 Ratio)

In a 1-L, 4-neck kettle equipped with a thermocouple, a condenser, a nitrogen purge adapter, and a mechanical stirrer, 122 g of amber-colored, viscous product from maleation reaction A2 was heated to 90° C. and added a mixture of 11.55 g (0.9 mole equivalent) of sodium carbonate and 24.6 g (2.1 mole equivalents) of calcium carbonate dissolved in 122 g (58 mole equivalents) of water over 3 hours controlling the foam formation. The reaction was held at 90° C. for 3 hours. The reaction yielded >90% flowable, light-yellow colored, 60% active, water-soluble product, which was characterized by IR and LC (<0.05% residual maleic anhydride).

### Example D3: Sodium Calcium Salt of Maleated Canola Oil from Calcium Carbonate and Sodium Carbonate (7:3 Ratio)

In a 1-L, 4-neck kettle equipped with a thermocouple, a condenser, a nitrogen purge adapter, and a mechanical stirrer, 122 g of amber-colored, viscous product from maleation reaction A3 was heated to 90° C., and a mixture of 11.3 g (0.9 mole equivalent) of sodium carbonate and 24 g (2.1 mole equivalents) of calcium carbonate dissolved in 122 g (60 mole equivalents) of water was added over 3 hours, controlling the foam formation. The reaction was held at 90° C. for 3 hours. The reaction yielded >90% flowable, light-yellow colored, 60% active, water-soluble product, which was characterized by IR and LC (<0.05% residual maleic anhydride).

### Example D4: Sodium Calcium Salt of Maleated Sunflower Oil from Calcium Carbonate and Sodium Carbonate (7:3 Ratio)

In a 1-L, 4-neck kettle equipped with a thermocouple, a condenser, a nitrogen purge adapter, and a mechanical stirrer, 122 g of amber-colored, viscous product from maleation reaction A4 was heated to 90° C., and a mixture of 11.55 g (0.9 mole equivalent) of sodium carbonate and 24.6 g (2.1 mole equivalents) of calcium carbonate dissolved in 122 g (58 mole equivalents) of water was added over 3 hours, controlling the foam formation. The reaction was held at 90° C. for 3 hours. The reaction yielded >90% flowable, light-yellow colored, 60% active, water-soluble product, which was characterized by IR and LC (<0.05% residual maleic anhydride).

### Example D5: Sodium Calcium Salt of Maleated Castor Oil from Calcium Carbonate and Sodium Carbonate (7:3 Ratio)

In a 1-L, 4-neck kettle equipped with a thermocouple, a condenser, a nitrogen purge adapter, and a mechanical stirrer, 122 g of amber-colored, viscous product from maleation reaction A5 was heated to 90° C., and a mixture of 10.6 g (0.9 mole equivalent) of sodium carbonate and 22.5 g (2.1 mole equivalents) of calcium carbonate dissolved in 122 g (63 mole equivalents) of water was added over 3 hours, controlling the foam formation. The reaction was held at 90° C. for 3 hours. The reaction yielded >90% flowable, light-yellow colored, 60% active, water-soluble product, which was characterized by IR and LC (<0.05% residual maleic anhydride).

### Example E1: Calcium Salt of Maleated Soybean Oil from Calcium Carbonate

In a 1-L, 4-neck kettle equipped with a thermocouple, a condenser, a nitrogen purge adapter, and a mechanical stirrer, 100 g of amber-colored, viscous product from maleation reaction A1 was heated to 90° C., and a mixture of 25.5 g (3 mole equivalents) of calcium carbonate in 85 g (55 mole equivalents) of water over 3 hours controlling the foam formation. The reaction was held at 90° C. or 3 hours. The reaction yielded >80% flowable, light-yellow colored, 60% active, water-soluble product, which was characterized by IR and LC (<0.05% residual maleic anhydride).

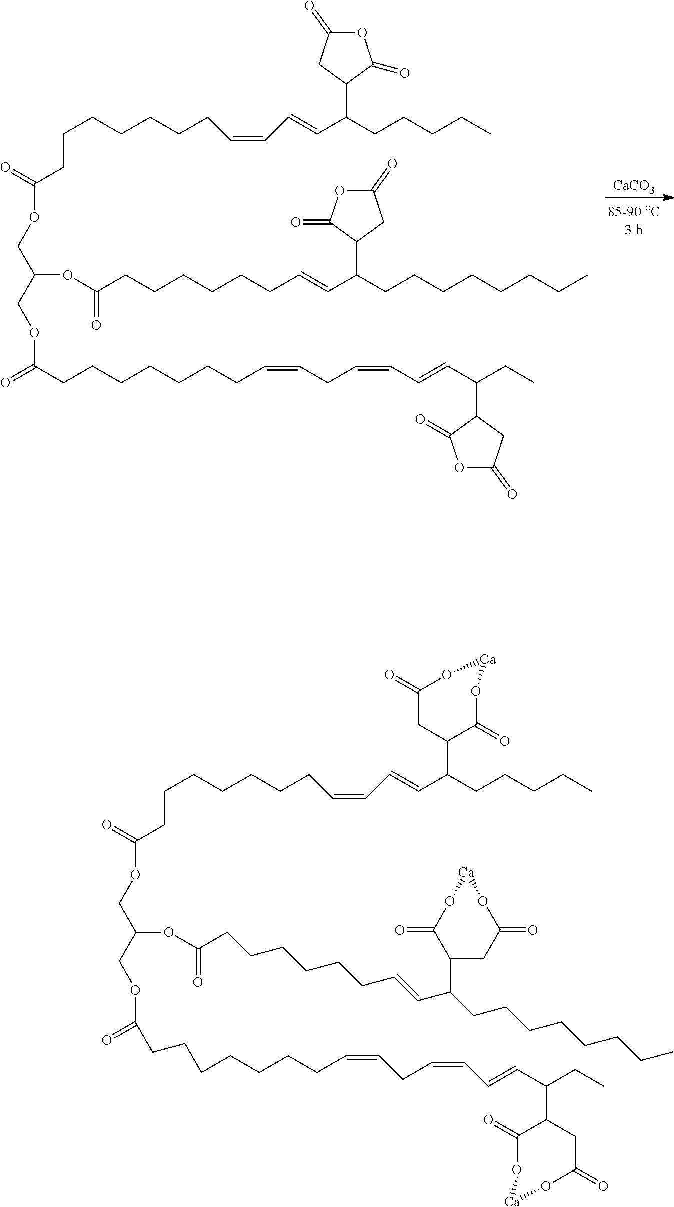

### Example F1: Sodium Salt of Maleated Soybean Oil from 2 Mole Equivalents of Aqueous Sodium Hydroxide

In a 1-L, 4-neck kettle equipped with a thermocouple, a condenser, a nitrogen purge adapter, and a mechanical stirrer, 123 g of amber-colored, viscous product from maleation reaction A1 was heated to 90° C., and a mixture of 9.04 g (2 mole equivalents) of 50% aqueous sodium hydroxide and 80 g (46 mole equivalents) of water was added over 1 hour, controlling foaming, and held at 90° C. for 3 hours. The reaction yielded >96% of a flowable, amber-colored, 70% active, water-soluble product, which was characterized by IR, NMR and LC (<0.05% residual maleic anhydride).

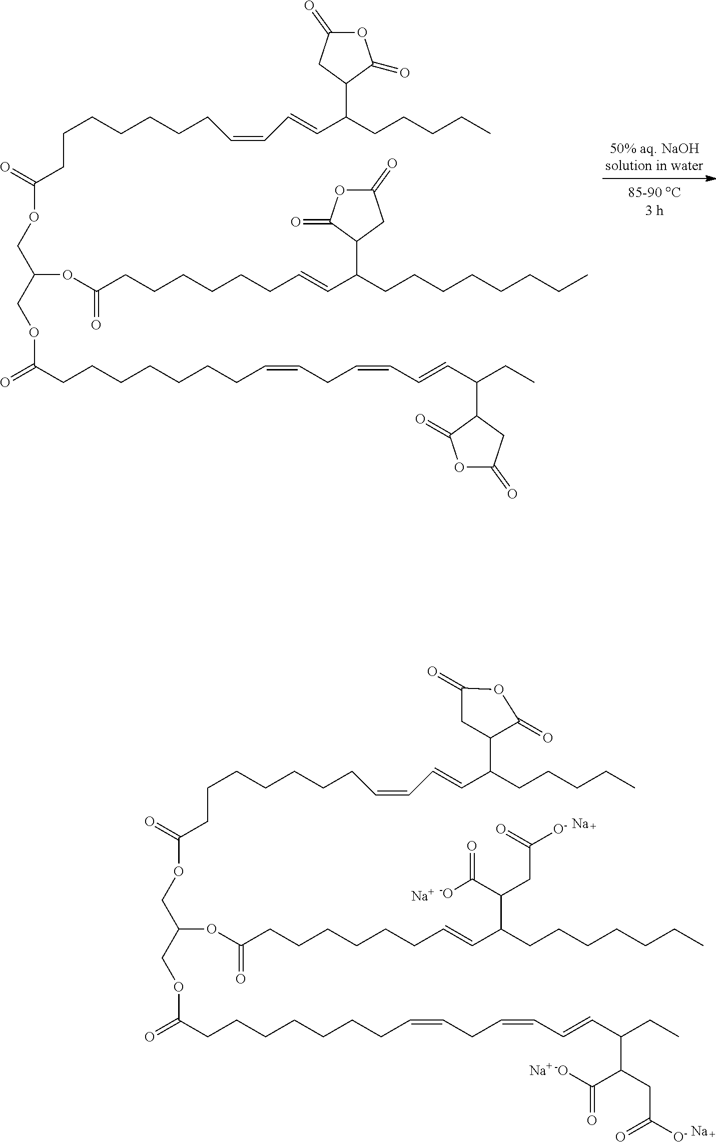

### Example G1: Sodium Salt of Maleated Soybean Oil Reacted with Hydrophobic 2-Octyl-1-Dodecanol

In a 1-L, 4-neck kettle equipped with a thermocouple, a condenser, a nitrogen purge adapter, and a mechanical stirrer, the amber-colored, viscous product from reaction F1 and 34 g (1 mole equivalent) of 2-octyl-1-dodecanol were mixed, heated to 90° C. and held there for 8 hours. The one-pot reaction without catalyst or solvent yielded >90% amber-colored, viscous product, which was characterized by NMR, IR, GC (<5% residual octyl dodecanol) and LC (<1% residual maleic anhydride).

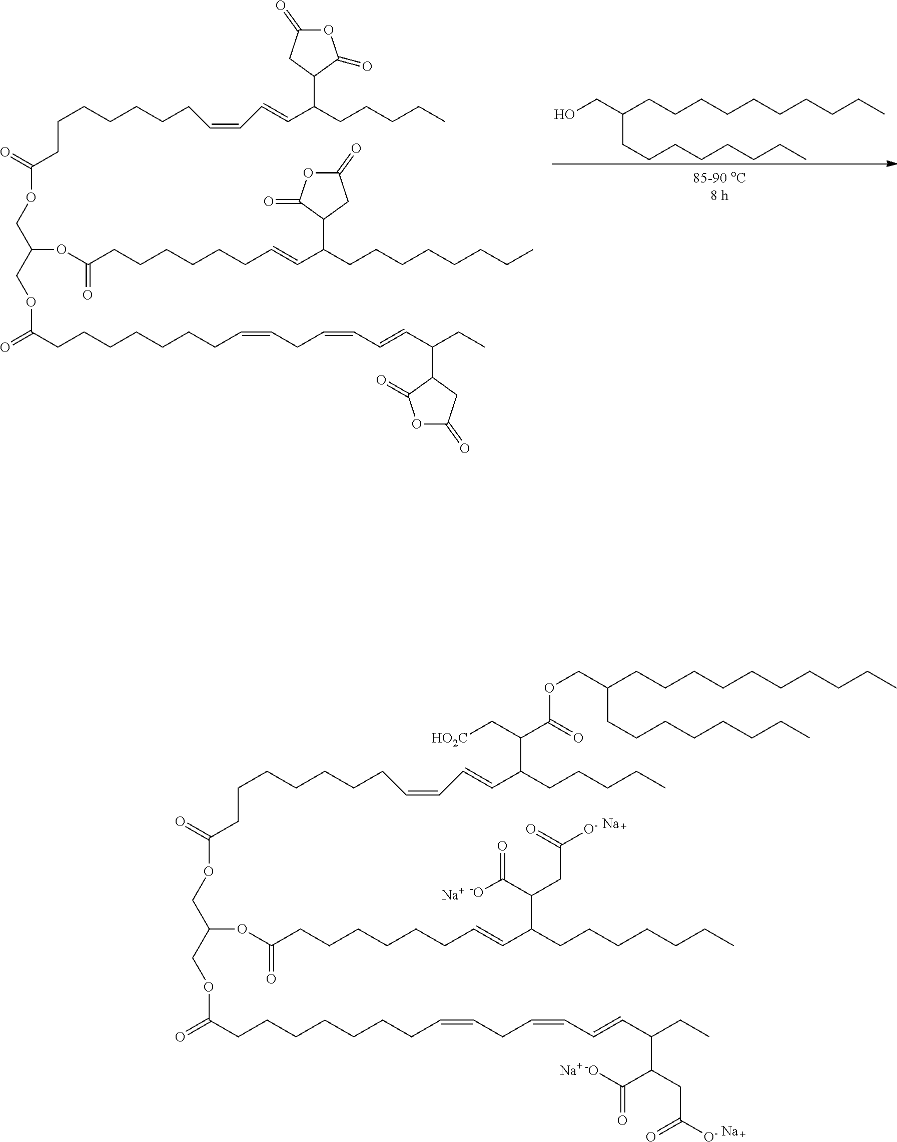

### Example H1: Sodium Salt of Maleated Soybean Oil Reacted with Hydrophilic Glycerin

In a 1-L, 4-neck kettle equipped with a thermocouple, a condenser, a nitrogen purge adapter, and a mechanical stirrer, the amber-colored, viscous product from reaction F1 and 10.5 g (1 mole equivalent) of glycerin were mixed, heated to 90° C. and held there for 4-6 hours. The one-pot reaction without catalyst or solvent yielded >90% amber-colored, viscous product, which was characterized by NMR and LC (<0.05% residual maleic anhydride).

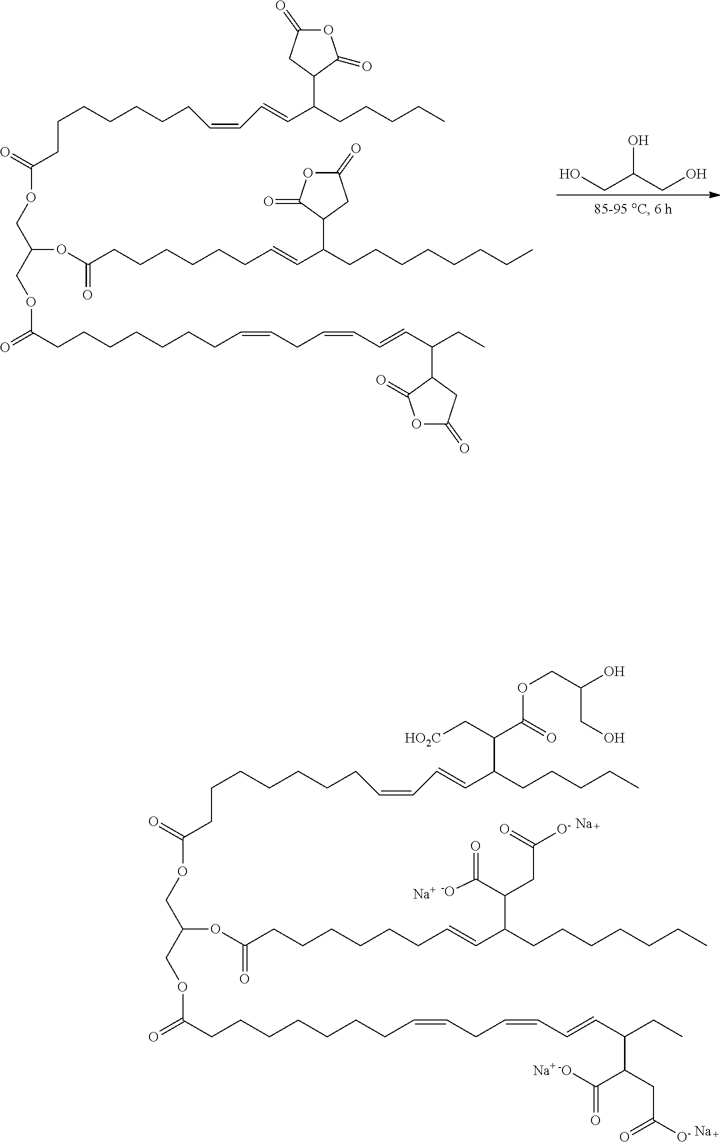

### Example I1: Sodium Salt of Maleated Soybean Oil Reacted with Hydrophobic 2-Octyl-1-Dodecanol and Hydrophilic Glycerin

A 1-L, 4-neck kettle equipped with a thermocouple, a condenser, a nitrogen purge adapter, and a mechanical stirrer was charged with 100 g of maleated soybean oil, produced as in Example A1, and 38.17 g (1.5 mole equivalents, relative to maleated soybean oil) of 2-octyl-1-dodecanol, and the mixture was heated at 90° C. for 6 hours. Glycerin (7.85 g, 1 mole equivalent, relative to maleated soybean oil) was added, and the mixture was heated at 90° C. for another 6 hours. The reaction mixture was cooled to 85° C., and 5.1 g (0.75 mole equivalent, relative to maleated soybean oil) of 50 wt % sodium hydroxide was added over 30 minutes, and the mixture was held at 85° C. for 4 hours. The amber, viscous product was characterized by NMR and IR. Yield was >96%.

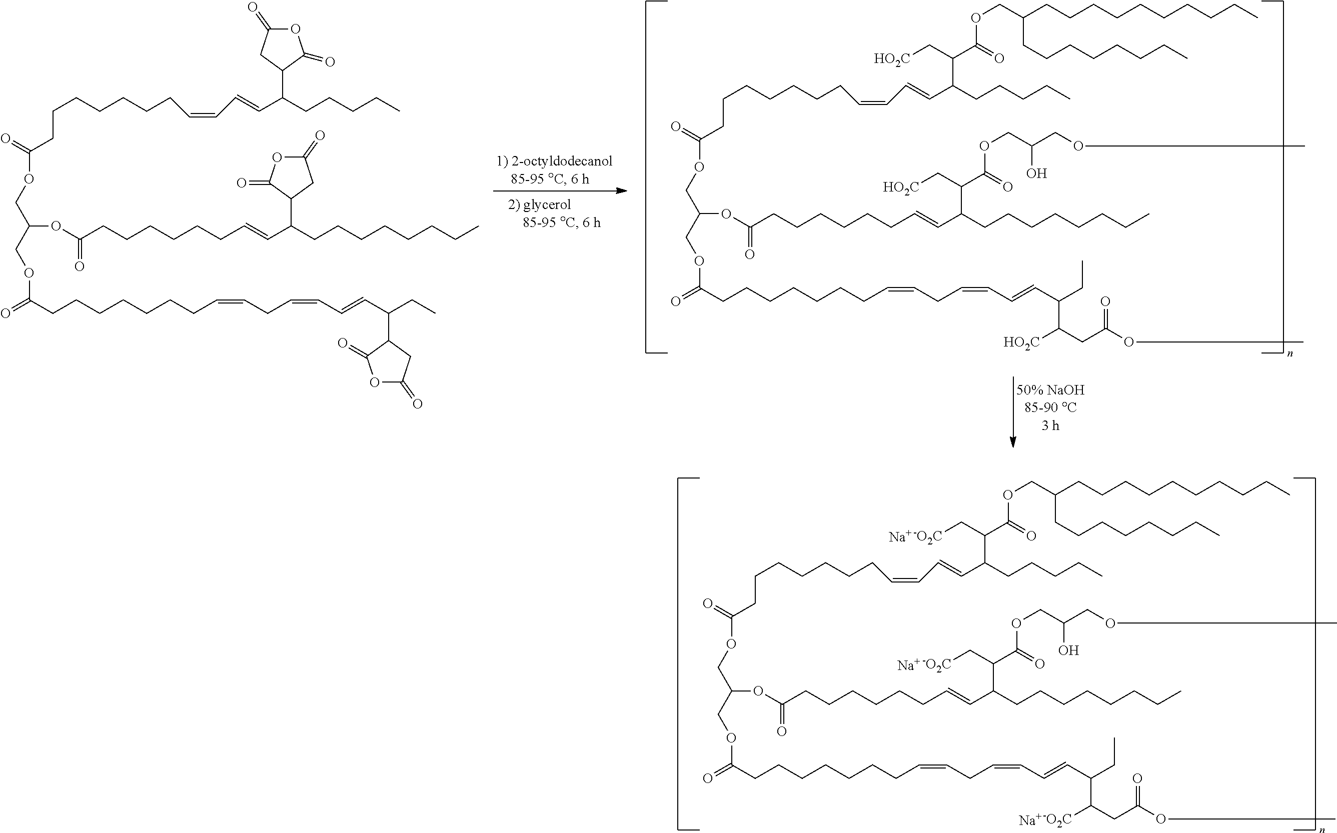

### Example J1: 3-Aminopropanol Salt (1 Equivalent) of Maleated Soybean Oil Reacted with Hydrophobic 2-Octyldodecanol and Hydrophilic Glycerin

A 1-L, 4-neck kettle equipped with a thermocouple, a reflux condenser, a nitrogen purge adapter, and a mechanical stirrer was charged with 100 g of maleated soybean oil, produced as in Example A1, and 38.17 g (1.5 mole equivalents, relative to maleated soybean oil) of 2-octyl-1-dodecanol, and the mixture was heated at 90° C. for 6 hours. Glycerin (7.85 g, 1 mole equivalent, relative to maleated soybean oil) was added, and the mixture was heated at 90° C. for another 6 hours. The reaction mixture was cooled to 60° C., and 1 mole equivalent of 3-amino-1-propanol was added over 30 minutes, and the mixture was held at 60° C. for 2 to 4 hours. The amber, viscous product was characterized by NMR and IR. Yield was >96%.

### Example J2: 3-Aminopropanol Salt (3 Equivalents) of Maleated Soybean Oil Reacted with Hydrophobic 2-Octyldodecanol and Hydrophilic Glycerin

A 1-L, 4-neck kettle equipped with a thermocouple, a reflux condenser, a nitrogen purge adapter, and a mechanical stirrer was charged with 100 g of maleated soybean oil, produced as in Example A1, and 38.17 g (1.5 mole equivalents, relative to maleated soybean oil) of 2-octyl-1-dodecanol, and the mixture was heated at 90° C. for 6 hours. Glycerin (7.85 g, 1 mole equivalent, relative to maleated soybean oil) was added, and the mixture was heated at 90° C. for another 6 hours. The reaction mixture was cooled to 60° C., and 3 mole equivalents of 3-amino-1-propanol was added over 30 minutes, and the mixture was held at 60° C. for 2 to 4 hours. The amber, viscous product was characterized by NMR and IR. Yield was >96%.

### Example J3: 3-Aminopropanol Salt of Maleated Soybean Oil Reacted with Hydrophobic 2-Octyldodecanol and Hydrophilic Glycerin, 50% Solids in Ethanol

In a 1-L, 4-neck kettle equipped with a thermocouple, reflux condenser, nitrogen purge adapter, and mechanical stirrer, 200 g of maleated soybean oil, produced as in Example A1, and 200 g of water were heated to 90° C. and held there for 6 hours. NMR and IR of the yellow, biphasic mixture confirmed hydrolysis. Water was removed by distillation. The thick amber mixture was dissolved in 200 g of ethanol at 50-60° C. Heating was discontinued, and, at 50° C. 65.4 g of 3-amino-1-propanol (5 mole equivalents, relative to soybean oil) was added over 1 hour, maintaining the internal temperature below 50° C. The mixture was held for 2 hours at room temperature. The amber, jelly-like product was 50% solids and was characterized by NMR and IR.

### Example J4: 3-Aminopropanol Salt of Maleated Soybean Oil Reacted with Hydrophobic 2-Octyldodecanol and Hydrophilic Glycerin, 30% Solids in Ethanol

The procedure of Example J3 was performed, except using 400 g of ethanol to dissolve the intermediate after removal of water by distillation. The clear amber product was 30% solids and was characterized by NMR and IR.

### Example K1: Maleated Soybean Oil Reacted with 0.5 Equivalent of 1-(2-Hydroxyethyl)-2-Pyrrolidone (HEP)

In a 1-L, 4-neck kettle equipped with a thermocouple, a reflux condenser, a nitrogen purge adapter, and a mechanical stirrer, 100 g of maleated soybean oil, produced as in Example A1, and 5.7 g (0.5 mole equivalent, relative to soybean oil) of HEP were mixed and heated to 120° C. and held there for 6 hours. The one-pot reaction without catalyst or solvent yielded >96% of amber, viscous product, which was characterized by NMR, IR, GC (<5% residual HEP) and LC (<0.5% residual maleic anhydride).

### Example K2: Maleated Soybean Oil Reacted with 1.0 Equivalent of HEP

The procedure of Example K1 was followed, except using 11.5 g (1.0 mole equivalent) of HEP.

### Example K3: Maleated Soybean Oil Reacted with 1.5 Equivalents of HEP

The procedure of Example K1 was followed, except using 17.2 g (1.5 mole equivalents) of HEP.

### Example K4: Maleated Soybean Oil Reacted with 2.0 Equivalents of HEP

The procedure of Example K1 was followed, except using 22.9 g (2.0 mole equivalents) of HEP.

### Example K5: Maleated Soybean Oil Reacted with 2.5 Equivalents of HEP

The procedure of Example K1 was followed, except using 28.7 g (2.5 mole equivalents) of HEP.

### Example K6: Maleated Soybean Oil Reacted with 3.0 Equivalents of HEP

The procedure of Example K1 was followed, except using 34.4 g (3.0 mole equivalents) of HEP.

### Example K7: Sodium Salt of Maleated Soybean Oil Reacted with 2.0 Equivalents of HEP

In a 1-L, 4-neck kettle equipped with a thermocouple, a reflux condenser, a nitrogen purge adapter, and a mechanical stirrer, 118 g of maleated soybean oil reacted with 2.0 equivalents of HEP, produced as in Example K4, was brought to 80° C., and a mixture of 20.5 g (3 mole equivalents, relative to soybean oil) of 50 wt % aqueous sodium hydroxide and 118 g of water was added over 30 minutes. The mixture was held at 85° C. for 2-4 hours. The amber, viscous crosslinked product was characterized by NMR and IR. Yield of the product was >96%.

### Example L1: Maleated Soybean Oil Reacted with 1.2 Equivalents of Hydrophobic 2-Ethylhexanol

In a 1-L, 4-neck kettle equipped with a thermocouple, reflux condenser, nitrogen purge adapter and mechanical stirrer, 100 g of maleated soybean oil, produced as in Example A1, was heated to 85° C., charged with 13.8 g (1.2 mole equivalents) of 2-ethyl hexanol and held at 85° C. for 6-8 hours. The amber liquid product was characterized by NMR and IR. Yield of the liquid product >98%.

### Example L2: Maleated Soybean Oil Reacted with 1.3 Equivalents of Hydrophobic 2-Ethylhexanol

The reaction was run as in Example L1, except using 14.9 g (1.3 mole equivalents) of 2-ethylhexanol.

### Example L3: Maleated Soybean Oil Reacted with 1.5 Equivalents of Hydrophobic 2-Ethylhexanol

The reaction was run as in Example L1, except using 17.2 g (1.5 mole equivalents) of 2-ethylhexanol.

### Example L4: Maleated Soybean Oil Reacted with 1.7 Equivalents of Hydrophobic 2-Ethylhexanol

The reaction was run as in Example L1, except using 19.5 g (1.7 mole equivalents) of 2-ethylhexanol.

### Example L5: Maleated Soybean Oil Reacted with 2.0 Equivalents of Hydrophobic 2-Ethylhexanol

The reaction was run as in Example L1, except using 22.9 g (2.0 mole equivalents) of 2-ethylhexanol.

### Example L6: Sodium Salt of Maleated Soybean Oil Reacted with 1.2 Equivalents of 2-Ethylhexanol and 1.3 Equivalents of Glycerin

In a 1-L, 4-neck kettle equipped with a thermocouple, reflux condenser, nitrogen purge adapter and mechanical stirrer, 100 g of maleated soybean oil reacted with 1.2 equivalents of hydrophobic 2-ethylhexanol, prepared as in Example L1, was heated to 85° C. and charged with 10.5 g (1.3 mole equivalents) of glycerin. The reaction mixture was held at 85° C. for 6-8 hours. The amber liquid intermediate was characterized by NMR and IR. Yield was >98%. The reaction mixture was cooled to 80° C., 4.5 g (0.75 mole equivalent, relative to soybean oil) of 50 wt % aqueous sodium hydroxide was added over 30 minutes, and the mixture was held at 85° C. for 2-4 hours. The amber, viscous crosslinked product was characterized by NMR and IR. Yield of the product was >96%.

### Example L7: Sodium Salt of Maleated Soybean Oil Reacted with 1.3 Equivalents of 2-Ethylhexanol and 1.2 Equivalents of Glycerin

In a 1-L, 4-neck kettle equipped with a thermocouple, reflux condenser, nitrogen purge adapter and mechanical stirrer, 100 g of maleated soybean oil reacted with 1.3 equivalents of hydrophobic 2-ethylhexanol, prepared as in Example L2, was heated to 85° C. and charged with 9.7 g (1.2 mole equivalents) of glycerin. The reaction mixture was held at 85° C. for 6-8 hours. The amber liquid intermediate was characterized by NMR and IR. Yield was >98%. The reaction mixture was cooled to 80° C., 4.5 g (0.75 mole equivalent, relative to soybean oil) of 50 wt % aqueous sodium hydroxide was added over 30 minutes, and the mixture was held at 85° C. for 2-4 hours. The amber, viscous crosslinked product was characterized by NMR and IR. Yield of the product was >96%.

### Example L8: Sodium Salt of Maleated Soybean Oil Reacted with 1.5 Equivalents of 2-Ethylhexanol and 1.0 Equivalent of Glycerin

In a 1-L, 4-neck kettle equipped with a thermocouple, reflux condenser, nitrogen purge adapter and mechanical stirrer, 100 g of maleated soybean oil reacted with 1.5 equivalents of hydrophobic 2-ethylhexanol, prepared as in Example L3, was heated to 85° C. and charged with 6.91 g (1.0 mole equivalents) of glycerin. The reaction mixture was held at 85° C. for 6-8 hours. The amber liquid intermediate was characterized by NMR and IR. Yield was >98%. The reaction mixture was cooled to 80° C., 4.5 g (0.75 mole equivalent, relative to soybean oil) of 50 wt % aqueous sodium hydroxide was added over 30 minutes, and the mixture was held at 85° C. for 2-4 hours. The amber, viscous crosslinked product was characterized by NMR and IR. Yield of the product was >96%.

### Example L9: Sodium Salt of Maleated Soybean Oil Reacted with 1.7 Equivalents of 2-Ethylhexanol and 1.0 Equivalent of Glycerin

In a 1-L, 4-neck kettle equipped with a thermocouple, reflux condenser, nitrogen purge adapter and mechanical stirrer, 100 g of maleated soybean oil reacted with 1.7 equivalents of hydrophobic 2-ethylhexanol, prepared as in Example L4, was heated to 85° C. and charged with 6.91 g (1.0 mole equivalents) of glycerin. The reaction mixture was held at 85° C. for 6-8 hours. The amber liquid intermediate was characterized by NMR and IR. Yield was >98%. The reaction mixture was cooled to 80° C., 4.5 g (0.75 mole equivalent, relative to soybean oil) of 50 wt % aqueous sodium hydroxide was added over 30 minutes, and the mixture was held at 85° C. for 2-4 hours. The amber, viscous crosslinked product was characterized by NMR and IR. Yield of the product was >96%.

### Example L10: Sodium Salt of Maleated Soybean Oil Reacted with 2.0 Equivalents of 2-Ethylhexanol and 0.5 Equivalent of Glycerin

In a 1-L, 4-neck kettle equipped with a thermocouple, reflux condenser, nitrogen purge adapter and mechanical stirrer, 100 g of maleated soybean oil reacted with 2.0 equivalents of hydrophobic 2-ethylhexanol, prepared as in Example L5, was heated to 85° C. and charged with 3.45 g (0.5 mole equivalents) of glycerin. The reaction mixture was held at 85° C. for 6-8 hours. The amber liquid intermediate was characterized by NMR and IR. Yield was >98%. The reaction mixture was cooled to 80° C., 4.5 g (0.75 mole equivalent, relative to soybean oil) of 50 wt % aqueous sodium hydroxide was added over 30 minutes, and the mixture was held at 85° C. for 2-4 hours. The amber, viscous crosslinked product was characterized by NMR and IR. Yield of the product was >96%.

### Example L11: Sodium Salt of Maleated Soybean Oil Reacted with 1.5 Equivalents of 2-Ethylhexanol, 1.25 Equivalents of Castor Oil and 0.5 Equivalent of Glycerin

In a 1-L, 4-neck kettle equipped with a thermocouple, reflux condenser, nitrogen purge adapter and mechanical stirrer, 100 g of maleated soybean oil reacted with 1.5 equivalents of hydrophobic 2-ethylhexanol, prepared as in Example L3, was heated to 85° C. and charged with 87.5 g (1.25 mole equivalents) of castor oil and 3.45 g (0.5 mole equivalents) of glycerin. The reaction mixture was held at 85° C. for 6-8 hours. The amber liquid intermediate was characterized by NMR and IR. Yield was >98%. The reaction mixture was cooled to 80° C., 4.5 g (0.75 mole equivalent, relative to soybean oil) of 50 wt % aqueous sodium hydroxide was added over 30 minutes, and the mixture was held at 85° C. for 2-4 hours. The amber, viscous crosslinked product was characterized by NMR and IR. Yield of the product was >96%.

### Example L12: Sodium Salt of Maleated Soybean Oil Reacted with 1.5 Equivalents of 2-Ethylhexanol, 0.5 Equivalent of Castor Oil and 0.5 Equivalent of Glycerin

In a 1-L, 4-neck kettle equipped with a thermocouple, reflux condenser, nitrogen purge adapter and mechanical stirrer, 100 g of maleated soybean oil reacted with 1.5 equivalents of hydrophobic 2-ethylhexanol, prepared as in Example L3, was heated to 85° C. and charged with 34.9 g (0.5 mole equivalent) of castor oil and 3.45 g (0.5 mole equivalents) of glycerin. The reaction mixture was held at 85° C. for 6-8 hours. The amber liquid intermediate was characterized by NMR and IR. Yield was >98%. The reaction mixture was cooled to 80° C., 4.5 g (0.75 mole equivalent, relative to soybean oil) of 50 wt % aqueous sodium hydroxide was added over 30 minutes, and the mixture was held at 85° C. for 2-4 hours. The amber, viscous crosslinked product was characterized by NMR and IR. Yield of the product was >96%.

### Example L13: Sodium Salt of Maleated Soybean Oil Reacted with 0.3 Equivalent of Castor Oil

In a 1-L, 4-neck kettle equipped with a thermocouple, reflux condenser, nitrogen purge adapter and mechanical stirrer, 152 g of maleated soybean oil, DS 2.9, produced as in Example A1, was heated to 85° C. and charged with 34.9 g (0.3 mole equivalent) of castor oil. The reaction mixture was held at 85° C. for 4-6 hours. The amber liquid intermediate was characterized by NMR. The reaction mixture was cooled to 80° C., a mixture of 30.3 g (2.9 mole equivalent, relative to soybean oil) of 50 wt % aqueous sodium hydroxide and 70 g of water was added over 30-45 minutes, and the mixture was held at 85° C. for 2-4 hours. The amber, viscous crosslinked product was characterized by NMR and IR. Yield of the product was >96%.

### Example L14: Sodium Salt of Maleated Soybean Oil Reacted with 0.3 Equivalent of Castor Oil and 1.5 equivalents of 2-ethylhexanol

In a 1-L, 4-neck kettle equipped with a thermocouple, reflux condenser, nitrogen purge adapter and mechanical stirrer, 100 g of maleated soybean oil, produced as in Example A1, was heated to 85° C. and charged with 24.8 g (0.3 mole equivalent) of castor oil. The reaction mixture was held at 85° C. for 4-6 hours. The amber liquid intermediate was characterized by NMR and IR. The reaction mixture was charged with 1.5 mole equivalent of 2-ethylhexanol and held at 85° C. for 6-8 hours. The reaction mixture was cooled to 80° C., 5.3 g (0.75 mole equivalent, relative to soybean oil) of 50 wt % aqueous sodium hydroxide was added over 30 minutes, and the mixture was held at 85° C. for 2-4 hours. The amber, viscous crosslinked product was characterized by NMR and IR. Yield of the product was >96%.

### Example M1: Sodium Salt of Maleated Soybean Oil Reacted with 1.5 Equivalents of 1-Hexanol and 1.0 Equivalent of Glycerin

In a 1-L, 4-neck kettle equipped with a thermocouple, reflux condenser, nitrogen purge adapter and mechanical stirrer, 200 g of maleated soybean oil, produced as in Example A1, was heated to 85° C. and charged with 26.8 g (1.5 mole equivalent) of 1-hexanol. The reaction mixture was held at 85° C. for 6-8 hours. The amber liquid intermediate was characterized by NMR and IR. The reaction mixture was charged with 16.1 g (1.0 mole equivalent) of glycerine and held at 85° C. for 6-8 hours. The reaction mixture was cooled to 80° C., 10.5 g (0.75 mole equivalent, relative to soybean oil) of 50 wt % aqueous sodium hydroxide was added over 30 minutes, and the mixture was held at 85° C. for 2-4 hours. The amber, viscous crosslinked product was characterized by NMR and IR. Yield of the product was >96%.

### Example M2: Sodium Salt of Maleated Soybean Oil Reacted with 1.5 Equivalents of 1-Dodecanol and 1.0 Equivalent of Glycerin

In a 1-L, 4-neck kettle equipped with a thermocouple, reflux condenser, nitrogen purge adapter and mechanical stirrer, 200 g of maleated soybean oil, produced as in Example A1, was heated to 85° C. and charged with 48.9 g (1.5 mole equivalent) of 1-dodecanol. The reaction mixture was held at 85° C. for 6-8 hours. The amber liquid intermediate was characterized by NMR and IR. The reaction mixture was charged with 16.1 g (1.0 mole equivalent) of glycerine and held at 85° C. for 6-8 hours. The reaction mixture was cooled to 80° C., 10.5 g (0.75 mole equivalent, relative to soybean oil) of 50 wt % aqueous sodium hydroxide was added over 30 minutes, and the mixture was held at 85° C. for 2-4 hours. The amber, viscous crosslinked product was characterized by NMR and IR. Yield of the product was >96%.

### Example M3: Sodium Salt of Maleated Soybean Oil Reacted with 1.5 Equivalents of 1-Eicosanol and 1.0 Equivalent of Glycerin

In a 1-L, 4-neck kettle equipped with a thermocouple, reflux condenser, nitrogen purge adapter and mechanical stirrer, 200 g of maleated soybean oil, produced as in Example A1, was heated to 85° C. and charged with 78.4 g (1.5 mole equivalent) of 1-eicosanol. The reaction mixture was held at 85° C. for 6-8 hours. The amber liquid intermediate was characterized by NMR and IR. The reaction mixture was charged with 16.1 g (1.0 mole equivalent) of glycerine and held at 85° C. for 6-8 hours. The reaction mixture was cooled to 80° C., 10.5 g (0.75 mole equivalent, relative to soybean oil) of 50 wt % aqueous sodium hydroxide was added over 30 minutes, and the mixture was held at 85° C. for 2-4 hours. The amber, viscous crosslinked product was characterized by NMR and IR. Yield of the product was >96%.

### Example N1: Sodium Salt of Maleated Soybean Oil Reacted with 1.0 Equivalent of Diethanolamine

In a 1-L, 4-neck kettle equipped with a thermocouple, reflux condenser, nitrogen purge adapter, and mechanical stirrer, 150 g of maleated soybean oil, produced as in Example A1, was heated to 60° C. To the maleated soybean oil was added, over 30-45 minutes, a solution of 15.1 g (1 mole equivalent) of diethanolamine in 300 g of water. The reaction mixture was held at 60° C. for 4-6 hours. The viscous, biphasic mixture was neutralized by adding 9.5 g (0.9 mole equivalent, relative to soybean oil) of 50 wt % aqueous sodium hydroxide over 15 minutes, and the mixture was held at 60° C. for 1-2 hours. The amber viscous product was characterized by NMR and IR. Yield was >96%.

### Example N2: Sodium Salt of Maleated Soybean Oil Reacted with 2.0 Equivalents of Diethanolamine

In a 1-L, 4-neck kettle equipped with a thermocouple, reflux condenser, nitrogen purge adapter, and mechanical stirrer, 150 g of maleated soybean oil, produced as in Example A1, was heated to 60° C. To the maleated soybean oil was added, over 30-45 minutes, a solution of 30.2 g (2 mole equivalents) of diethanolamine in 300 g of water. The reaction mixture was held at 60° C. for 4-6 hours. The viscous, biphasic mixture was neutralized by adding 9.5 g (0.9 mole equivalent, relative to soybean oil) of 50 wt % aqueous sodium hydroxide over 15 minutes, and the mixture was held at 60° C. for 1-2 hours. The amber viscous product was characterized by NMR and IR. Yield was >96%.

### Example O1: Maleated Soybean Oil Reacted with Poly(Ethylene Glycol) Monomethyl Ether (mPEG), MW 1000

A 1-L, 4-neck kettle equipped with a thermocouple, reflux condenser, nitrogen purge adapter and mechanical stirrer was charged with 271.59 g of maleated soybean oil, produced as in Example A1. The maleated soybean oil was heated to 60° C., stirring was commenced, and 235.23 g (1.0 mole equivalent) of poly(ethylene glycol) monomethyl ether, MW 1000, (mPEG 1000) was added, maintaining the internal temperature below 90° C. Once the addition of mPEG was complete, the reaction mixture was held at 90° C. for 15 minutes, heated to 120° C. and held at 120° C. for 6 hours.

### Example O2: Sodium Salt of Maleated Soybean Oil Reacted with mPEG 1000

In a 500-mL, 4-neck kettle equipped with a thermocouple, reflux condenser, nitrogen purge adapter and mechanical stirrer, 142 g of maleated soybean oil reacted with mPEG 1000, produced according to Example O1, was heated to 80° C., and 12.13 g of 50 wt % aqueous sodium hydroxide was added. The mixture was held at 80° C. for 3 hours. The product had a pH between 6.5 and 7.5 and was characterized by IR.

### Example O3: Sodium Salt of Maleated Soybean Oil Reacted with mPEG 1000 and 2-Butyl-1-Octanol

In a 500-mL, 4-neck kettle equipped with a thermocouple, reflux condenser, nitrogen purge adapter and mechanical stirrer, 140 g of maleated soybean oil reacted with mPEG 1000, produced according to Example O1, was heated to 60° C., and 12.07 g (1 mole equivalent) of 2-butyl-1-octanol was added slowly, maintaining the internal temperature below 90° C. Once the addition of 2-butyl-1-octanol was complete, the reaction mixture was held at 90° C. for 15 minutes, heated to 120° C. and held at 120° C. for 6 hours. The reaction mixture was cooled to 80° C., 9.33 g of 50 wt % aqueous sodium hydroxide was added, and the mixture was held at 80° C. for 3 hours. The product had a pH between 6.5 and 7.5 and was characterized by NMR and IR.

### Example O4: Sodium Salt of Maleated Soybean Oil Reacted with mPEG 1000 and 1-Dodecanol

In a 500-mL, 4-neck kettle equipped with a thermocouple, reflux condenser, nitrogen purge adapter and mechanical stirrer, 140 g of maleated soybean oil reacted with mPEG 1000, produced according to Example O1, was heated to 60° C., and 12.07 g (1 mole equivalent) of 1-dodecanol was added slowly, maintaining the internal temperature below 90° C. Once the addition of 1-dodecanol was complete, the reaction mixture was held at 90° C. for 15 minutes, heated to 120° C. and held at 120° C. for 6 hours. The reaction mixture was cooled to 80° C., 9.33 g of 50 wt % aqueous sodium hydroxide was added, and the mixture was held at 80° C. for 3 hours. The product had a pH between 6.5 and 7.5 and was characterized by NMR and IR.

### Example O5: Maleated Soybean Oil Reacted with Poly(Ethylene Glycol) Monomethyl Ether (mPEG), MW 400

A 1-L, 4-neck kettle equipped with a thermocouple, reflux condenser, nitrogen purge adapter and mechanical stirrer was charged with 262.4 g of maleated soybean oil, produced as in Example A1. The maleated soybean oil was heated to 60° C., stirring was commenced, and 90.91 g (1.0 mole equivalent) of poly(ethylene glycol) monomethyl ether, MW 400, (mPEG 400) was added, maintaining the internal temperature below 90° C. Once the addition of mPEG was complete, the reaction mixture was held at 90° C. for 15 minutes, heated to 120° C. and held at 120° C. for 6 hours.

### Example O6: Sodium Salt of Maleated Soybean Oil Reacted with mPEG 400

A 500-mL, 4-neck kettle equipped with a thermocouple, reflux condenser, nitrogen purge adapter and mechanical stirrer was charged with 206.8 g of maleated soybean oil, produced as in Example A1. The maleated soybean oil was heated to 60° C., stirring was commenced, and 105.7 g (1.5 mole equivalent) of mPEG 400 was added, maintaining the internal temperature below 80° C. Once the addition of mPEG was complete, the reaction mixture was held at 80° C. for 15 minutes, heated to 90° C. and held at 90° C. for 6 hours. The reaction mixture was cooled to 80° C., and 31.7 g of 50 wt % aqueous sodium hydroxide was added. The mixture was held at 80° C. for 3 hours. The product had a pH between 6.5 and 7.5 and was characterized by IR.

### Example O7: Sodium Salt of Maleated Soybean Oil Reacted with mPEG 400 and 2-Butyl-1-Octanol

In a 500-mL, 4-neck kettle equipped with a thermocouple, reflux condenser, nitrogen purge adapter and mechanical stirrer, 132.5 g of maleated soybean oil reacted with mPEG 400, produced according to Example O5, was heated to 60° C., and 15.88 g (1 mole equivalent) of 2-butyl-1-octanol was added slowly, maintaining the internal temperature below 90° C. Once the addition of 2-butyl-1-octanol was complete, the reaction mixture was held at 90° C. for 15 minutes, heated to 120° C. and held at 120° C. for 6 hours. The reaction mixture was cooled to 80° C., 12.27 g of 50 wt % aqueous sodium hydroxide was added, and the mixture was held at 80° C. for 3 hours. The product had a pH between 6.5 and 7.5 and was characterized by NMR and IR.

### Example O8: Sodium Salt of Maleated Soybean Oil Reacted with mPEG 400 and 1-Dodecanol

In a 500-mL, 4-neck kettle equipped with a thermocouple, reflux condenser, nitrogen purge adapter and mechanical stirrer, 118.4 g of maleated soybean oil reacted with mPEG 400, produced according to Example O5, was heated to 60° C., and 14.2 g (1 mole equivalent) of 1-dodecanol was added slowly, maintaining the internal temperature below 90° C. Once the addition of 1-dodecanol was complete, the reaction mixture was held at 90° C. for 15 minutes, heated to 120° C. and held at 120° C. for 6 hours. The reaction mixture was cooled to 80° C., 10.96 g of 50 wt % aqueous sodium hydroxide was added, and the mixture was held at 80° C. for 3 hours. The product had a pH between 6.5 and 7.5 and was characterized by NMR and IR.

### Example O9: Sodium Salt of Maleated Soybean Oil Reacted with 2-Butyl-1-Octanol

A 1-L, 4-neck kettle equipped with a thermocouple, reflux condenser, nitrogen purge adapter and mechanical stirrer was charged with 242.7 g of maleated soybean oil, produced as in Example A1. The maleated soybean oil was heated to 60° C., stirring was commenced, and 57.7 g (1.5 mole equivalent) of 2-butyl-1-octanol was added, maintaining the internal temperature below 80° C. Once the addition of 2-butyl-1-octanol was complete, the reaction mixture was held at 80° C. for 15 minutes, heated to 90° C. and held at 90° C. for 6 hours. The reaction mixture was cooled to 80° C., 37.1 g of 50 wt % aqueous sodium hydroxide was added, and the mixture was held at 80° C. for 3 hours. The product had a pH between 6.5 and 7.5 and was characterized by NMR and IR.

### Example O10: Sodium Salt of Maleated Soybean Oil Reacted with 1-Dodecanol

A 1-L, 4-neck kettle equipped with a thermocouple, reflux condenser, nitrogen purge adapter and mechanical stirrer was charged with 205.5 g of maleated soybean oil, produced as in Example A1. The maleated soybean oil was heated to 60° C., stirring was commenced, and 48.91 g (1.5 mole equivalent) of 1-dodecanol was added, maintaining the internal temperature below 80° C. Once the addition of 2-butyl-1-octanol was complete, the reaction mixture was held at 80° C. for 15 minutes, heated to 90° C. and held at 90° C. for 6 hours. The reaction mixture was cooled to 80° C., 31.5 g of 50 wt % aqueous sodium hydroxide was added, and the mixture was held at 80° C. for 3 hours. The product had a pH between 6.5 and 7.5 and was characterized by NMR and IR.

### Examples for Modified Soyabean Oil in Agrochemical Formulations

### Example 1: Seed Coating

The compositions as shown in below tables were prepared by weighing in appropriate quantities of the ingredients to make 10 g samples in a 2-ounce stoppered bottle. The contents were dispersed using a magnetic stir bar over a period of 1 hour.

TABLE 1
Seed treatment composition

| Ingredient | % (w/w) | Function |
|---|---|---|
| Tebuconazole | 10.0 | Active Ingredient |
| Composite of PVP (polyvinyl | 1.55 | Dispersant |
| pyrrolidone) and MVE-MAHE | | |
| (Methyl Vinyl Ether/Maleic | | |
| Acid Half Ester | | |
| Xanthan Gum | 0.075 | Rheology Modifier |
| Propylene Glycol | 1.75 | Anti-Freeze |
| paraffin oil based with | 0.1 | Defoamer |
| silica | | Film |
| Polymer | 5.0 | former/Binder |
| aqueous pigment dispersion | 2.5 | Colorant |
| (60% solids) | | |
| Water | 79.025 | Solvent |

TABLE 2
Seed treatment composition including film former/binder polymer

| | Samp 1 | Samp 2 | Samp 3 | Samp 4 | Samp 5 |
|---|---|---|---|---|---|
| Ingredient | Wt | Wt | Wt | Wt | Wt |
| Water | 4.75 | 3.75 | 4.25 | 4.25 | 2.25 |
| Tebuconazole Seed | 5 | 5 | 5 | 5 | 5 |
| Treatment (20%) | | | | | |
| Styrene acrylic | — | 1 | — | — | — |
| copolymer | | | | | |
| Na salt Hydrophobically | — | — | 0.5 | — | — |
| modified maleated | | | | | |
| soybean oil 1 | | | | | |
| Na salt Hydrophobically | — | — | — | 0.5 | — |
| modified maleated | | | | | |
| soybean oil 2 | | | | | |
| PVOH 87-90% | — | — | — | — | 2.5 |
| 30-70K (20%) | | | | | |
| aqueous pigment | 0.25 | 0.25 | 0.25 | 0.25 | 0.25 |
| dispersion | | | | | |
| Total | 10 | 10 | 10 | 10 | 10 |

Corn seeds were coated with 1 g of the seed treatment formulation per 100 g seed using a Winterstieger Hege 11 liquid seed treater. The treatment was applied over 30 seconds and mixed for an additional 20 seconds to dry. Seeds were left in an open container at ambient temperature for 24 hours before dust-off was performed.

A Huebach Dustmeter operating for 300 seconds with 20 L/min air low rate and 30 rotations/minute was used to evaluate dust-off values.

TABLE 3
Corn seed Dust-off Data:

| | Dust-off |
|---|---|
| | (g per 250 g |
| Polymer | seeds) |
| Control (No Polymer) | 0.0079 |
| Styrene acrylic | 0.0004 |
| PVOH | 0.0020 |
| Na salt Hydrophobically modified | 0.0007 |
| maleated soybean oil #1 | |
| Na salt Hydrophobically modified | 0.0005 |
| maleated soybean oil #2 | |

### Example 2: Seed Treatment on Corn and Soybean

Soybean seeds were coating with a commercial seed treatment with a secondary addition of various binders and film formers to evaluate the performance of this preparation. Soybeans were treated with Acceleron DC309 at 0.375 fl oz/100 lbs, Acceleron DX 109 at 0.600 fl oz/100 lbs, Acceleron DX-612 at 0.240 fl oz/100 lbs, and Acceleron IX409 at 3.000 fl oz/100 lbs. Corn seeds were coated with Acceleron DC-309 at 0.100 fl oz/100 lbs, Accleron DC-509 at 0.085 fl oz/100 lbs, Acceleron DX-709 at 0.320 fl oz/100 lbs, and Acceleron IC-609 at 5.309 fl oz/100 lbs.

Dustoff performance was recorded using a Huebach Dustmeter on 100 g samples of seeds.

TABLE 4
Corn and Soybean Seed Treatment Dust off Data

| | | | Dust | g of Dust/ | Dust |
|---|---|---|---|---|---|
| | | | Amt per | 100,000 | Amt per |
| | | | 100 g - | Seed - | 100 Kg - |
| Sample | Description | Crop | Mean | Mean | mean |
| 1 | Untreated Control | Corn | 0.00077 | 0.17544 | 0.77000 |
| 2 | Standard Base Treated | Corn | 0.00164 | 0.37078 | 1.64333 |
| | (Std) | | | | |
| 3 | Std + Hydrophobically | Corn | 0.00031 | 0.07164 | 0.31333 |
| | modified maleated | | | | |
| | soybean oil #1 | | | | |
| 4 | Std + Hydrophobically | Corn | 0.00039 | 0.08935 | 0.39333 |
| | modified maleated | | | | |
| | soybean oil #2 | | | | |
| 5 | Std + Polyvinyl Alcohol | Corn | 0.00109 | 0.25135 | 1.09000 |
| 6 | Std + PVP/VA | Corn | 0.00186 | 0.42833 | 1.86333 |
| 7 | Std + Styrene acrylic | Corn | 0.00055 | 0.12501 | 0.54667 |
| 8 | Untreated Control | Soybean | 0.00052 | 0.08226 | 0.52000 |
| 9 | Standard Base Treated (Std) | Soybean | 0.00057 | 0.09020 | 0.57000 |
| 10 | Std + Hydrophobically | Soybean | 0.00041 | 0.06477 | 0.41000 |
| | modified maleated | | | | |
| | soybean oil #1 | | | | |
| 11 | Std + Hydrophobically | Soybean | 0.00038 | 0.05948 | 0.37667 |
| | modified maleated | | | | |
| | soybean oil #2 | | | | |
| 12 | Std + Polyvinyl Alcohol | Soybean | 0.00044 | 0.06860 | 0.44000 |
| 13 | Std + PVP/VA | Soybean | 0.00041 | 0.06382 | 0.41000 |
| 14 | Std + Styrene acrylic | Soybean | 0.00046 | 0.07113 | 0.46333 |

Plantability was measured on treated corn and soybean seeds. Corn plantability was tested using a 33,000 seed/acre planting rate at 77.4 C and 4400 relative humidity. Soybean plantability was tested using 150,000 seed/acre planting rate at 77.4 C and 44% relative humidity.

TABLE 5
Corn and Soybean seed treatment plantability data

| Treat- | | | | | | | | | |
|---|---|---|---|---|---|---|---|---|---|
| ment | | | | % | % | % | % | % | % |
| # | Description | Crop | Rep # | Singulation | Skips | Multiples | Singulation | Skips | Multiples |
| 1 | Untreated | Corn | Run 1 | 95.3 | 4.3 | 0.4 | 95.47 | 4.17 | 0.37 |
| | Control | | Run 2 | 95.1 | 4.6 | 0.3 | | | |
| | | | Run 3 | 96 | 3.6 | 0.4 | | | |
| 2 | Standard | Corn | Run 1 | 92.1 | 7.8 | 0.1 | 91.67 | 8.07 | 0.27 |
| | Base | | Run 2 | 91.5 | 8.2 | 0.3 | | | |
| | Treated | | Run 3 | 91.4 | 8.2 | 0.4 | | | |
| | (Std) | | | | | | | | |
| 3 | Std + | Corn | Run 1 | 93.2 | 6.3 | 0.5 | 92.23 | 7.2 | 0.57 |
| | Hydrophobically | | Run 2 | 91.5 | 8 | 0.5 | | | |
| | modified | | Run 3 | 92 | 7.3 | 0.7 | | | |
| | maleated | | | | | | | | |
| | soybean oil #1 | | | | | | | | |
| 4 | Std + | Corn | Run 1 | 98 | 1.9 | 0.1 | 98.83 | 1.07 | 0.1 |
| | Hydrophobically | | Run 2 | 99.2 | 0.8 | 0 | | | |
| | modified | | Run 3 | 99.3 | 0.5 | 0.2 | | | |
| | maleated | | | | | | | | |
| | soybean oil #2 | | | | | | | | |
| 5 | Std + | Corn | Run 1 | 95.7 | 2.7 | 1.6 | 95.8 | 2.83 | 1.37 |
| | polyvinyl | | Run 2 | 96.2 | 2.8 | 1 | | | |
| | alcohol | | Run 3 | 95.5 | 3 | 1.5 | | | |
| 6 | Std + PVP/VA | Corn | Run 1 | 93.5 | 6.3 | 0.2 | 93.7 | 5.93 | 0.37 |
| | copolymer | | Run 2 | 92.7 | 6.7 | 0.6 | | | |
| | | | Run 3 | 94.9 | 4.8 | 0.3 | | | |
| 7 | Std + Styrene | Corn | Run 1 | 93.9 | 5.9 | 0.2 | 93.43 | 6.27 | 0.3 |
| | Acrylic | | Run 2 | 93.4 | 6.1 | 0.5 | | | |
| | copolymer | | Run 3 | 93 | 6.8 | 0.2 | | | |
| 8 | Untreated | Soybean | Run 1 | 93.7 | 5.9 | 0.4 | 93.17 | 6.43 | 0.4 |
| | Control | | Run 2 | 93.7 | 6 | 0.3 | | | |
| | | | Run 3 | 92.1 | 7.4 | 0.5 | | | |
| 9 | Standard Base | Soybean | Run 1 | 54.8 | 42.8 | 2.4 | 51.17 | 46.9 | 1.93 |
| | Treated (Std) | | Run 2 | 51.3 | 46.7 | 2 | | | |
| | | | Run 3 | 47.4 | 51.2 | 1.4 | | | |
| 10 | Std + | Soybean | Run 1 | 95.9 | 3.8 | 0.3 | 95.13 | 4.4 | 0.47 |
| | Hydrophobically | | Run 2 | 94.4 | 5.1 | 0.5 | | | |
| | modified | | Run 3 | 95.1 | 4.3 | 0.6 | | | |
| | maleated | | | | | | | | |
| | soybean oil #1 | | | | | | | | |
| 11 | Std + | Soybean | Run 1 | 97 | 3 | 0 | 97.53 | 2.47 | 0 |
| | Hydrophobically | | Run 2 | 97.7 | 2.3 | 0 | | | |
| | modified | | Run 3 | 97.9 | 2.1 | 0 | | | |
| | maleated | | | | | | | | |
| | soybean oil #2 | | | | | | | | |
| 12 | Std + polyvinyl | Soybean | Run 1 | 91.9 | 7.8 | 0.3 | 89.3 | 9.4 | 1.3 |
| | alcohol | | Run 2 | 89.5 | 9 | 1.5 | | | |
| | | | Run 3 | 86.5 | 11.4 | 2.1 | | | |
| 13 | Std + PVP/VA | Soybean | Run 1 | 76.7 | 22.4 | 0.9 | 71.8 | 26.33 | 1.87 |
| | copolymer | | Run 2 | 69.8 | 27 | 3.2 | | | |
| | | | Run 3 | 68.9 | 29.6 | 1.5 | | | |
| 14 | Std + Styrene | Soybean | Run 1 | 99 | 0.8 | 0.2 | 99.17 | 0.5 | 0.33 |
| | Acrylic | | Run 2 | 99.2 | 0.6 | 0.2 | | | |
| | copolymer | | Run 3 | 99.3 | 0.1 | 0.6 | | | |

### Example 3: Preparation of Matrix

The compositions as shown below tables were prepared by weighing in appropriate quantities of the ingredients to make 100 g samples in a 4-ounce stoppered bottle. The contents were dispersed using an overhead mixer over a period of 1 hour.

100 g matrix composition was prepared by dispersing the following ingredients in a 4-ounce stoppered bottle. 3.75 g of Na Salt of Hydrophobically modified soybean oil, 2.5 g of Composite of PVP (polyvinyl pyrrolidone) and MVE-MAHE (Methyl Vinyl Ether/Maleic Acid Half Ester Neutralized) copolymer, 12.5 g of Phosphate ester, 5.0 g of Methylated soybean oil, and 73.75 g of Paraffinic oil. 2.5 g of bentonite was added last to adjust the final liquid system viscosity. The composition was held at 54 C for 14 days and remained visually stable, no separation of phases was observed.

TABLE 6
Matrix composition

| Ingredients | Wt. % |
|---|---|
| Na Salt of Hydrophobically | |
| Modified Maleated | |
| Soybean Oil | 3.75 |
| Composite of PVP and MVE-MAHE | 2.50 |
| Phosphate ester | 12.50 |
| Methylated soybean oil | 5.00 |
| Bentonite | 2.50 |
| Paraffinic oil | 73.75 |

### Example 4: Addition of Active Ingredient Atrazine

20.0 g of Atrazine was added to 78.0 g of the matrix (without Bentone SD-1) in example 1. 2.0 g of bentonite was slowly added to adjust the final liquid system viscosity.

TABLE 7
Atrazine composition

| Ingredients | Wt. grams |
|---|---|
| Atrazine | 20.0 |
| Hydrophobically modified maleated | 0.75 |
| soybean oil | |
| Na Salt of maleated soybean oil | 2.25 |
| Composite of PVP and MVE-MAHE | 2.0 |
| Phosphate ester | 10.0 |
| Methylated soybean oil | 5.0 |
| Bentonite | 2.0 |
| Paraffinic oil | 59.0 |

20.0 g of Atrazine was added to 78.0 g of the matrix (without Bentone SD-1) in example 1. 2.0 g of bentonite was slowly added to adjust the final liquid system viscosity.

TABLE 8
Atrazine composition

| Ingredients | Wt. grams |
|---|---|
| Atrazine | 20.0 |
| Na Salt of Hydrophobically modified | 3.0 |
| maleated soybean oil | |
| Composite of PVP and MVE-MAHE | 2.0 |
| Phosphate ester | 10.0 |
| Methylated soybean oil | 5.0 |
| Bentonite | 2.0 |
| Paraffinic oil | 59.0 |

### Example 5: Seed Coating

The compositions as shown in below tables is prepared by weighing in appropriate quantities of the ingredients to make 100 g samples in a 4-ounce stoppered bottle. The contents are dispersed using a magnetic stir bar over a period of 1 hour.

TABLE 9
Seed Coating

| Ingredients | Wt. grams |
|---|---|
| Tebuconazole SC (20%) | 10 |
| Composite of PVP (polyvinyl | 1.55 |
| pyrrolidone) and MVE-MAHE | |
| (Methyl Vinyl Ether/Maleic | |
| Acid Half Ester | |
| Xanthan Gum | 0.075 |
| Propylene Glycol | 1.75 |
| Paraffin oil based with silica | 0.1 |
| agent | |
| Polyvinyl Alcohol | 4 |
| Na salt Hydrophobically modified | 1 |
| maleated soybean oil | |
| aqueous pigment dispersion | 2.5 |
| (60% solids) | |
| Water | 79.025 |

### Example 6: Seed Coating

TABLE 10
Seed Coating

| Ingredients | Wt. grams |
|---|---|
| Tebuconazole SC (20%) | 10 |
| Composite of PVP (polyvinyl | 1.55 |
| pyrrolidone) and MVE-MAHE | |
| (Methyl Vinyl Ether/Maleic | |
| Acid Half Ester | |
| Xanthan Gum | 0.075 |
| Propylene Glycol | 1.75 |
| Paraffin oil based with silica | 0.1 |
| agent | |
| Polyethylene Glycol 400 | 3 |
| Na salt Hydrophobically modified | 2 |
| maleated soybean oil | |
| aqueous pigment dispersion | 2.5 |
| (60% solids) | |
| Water | 79.025 |

### Example 7: Seed Coating

TABLE 11
Seed Coating

| Ingredients | Wt. grams |
|---|---|
| Tebuconazole SC (20%) | 10 |
| Composite of PVP (polyvinyl | 1.55 |
| pyrrolidone) and MVE-MAHE | |
| (Methyl Vinyl Ether/Maleic | |
| Acid Half Ester | |
| Xanthan Gum | 0.075 |
| Propylene Glycol | 1.75 |
| Paraffin oil based with silica | 0.1 |
| agent | |
| Styrene acrylic copolymer | 2.5 |
| Na salt Hydrophobically modified | 2.5 |
| maleated soybean oil | |
| aqueous pigment dispersion | 2.5 |
| (60% solids) | |
| Water | 79.025 |

### Example 8: Seed Coating

TABLE 12
Seed Coating

| Ingredients | Wt. grams |
|---|---|
| Tebuconazole SC (20%) | 10 |
| Composite of PVP (polyvinyl pyrrolidone) | 1.55 |
| and MVE-MAHE (Methyl Vinyl Ether/ | |
| Maleic Acid Half Ester | |
| Xanthan Gum | 0.075 |
| Propylene Glycol | 1.75 |
| Paraffin oil based with silica agent | 0.1 |
| Vinyl pyrrolidone/vinyl acetate copolymer | 3 |
| Na salt Hydrophobically modified maleated | 2 |
| soybean oil | |
| aqueous pigment dispersion (60% solids) | 2.5 |
| Water | 79.025 |

### Example 9: Seed Coating

100 g seed coating composition is prepared in a 250 ml glass beaker. 10.0 g of Sodium Salt of maleated soybean oil in 45.56 g water, 12.00 g propylene glycol is added to beaker then combined via overhead mixer with a propellor mixing blade at 400 rpm for 10 minutes. 0.25 g Xanthan Gum added slowly and mixed for 15 minutes or until fully hydrated Composite of PVP (polyvinyl pyrrolidone) and MVE-MAHE (Methyl Vinyl Ether/Maleic Acid Half Ester Neutralized) copolymer, Silicone Defoamer and 20% aqueous dipropylene glycol solution of 1,2-benzisothiazolin-3-oneGXL added with mixing. Mixing speed increased to 600 rpm, then Zinc oxide, Zinc EDTA, and Sodium Molybdate and Red Pigment added slowly and mixed for 15 minutes.

TABLE 13
Seed Coating

| Ingredients | Wt. grams |
|---|---|
| Zinc Oxide | 25.0 |
| Sodium Molybdate | 0.26 |
| Zinc EDTA | 3.33 |
| Xanthan Gum | 0.25 |
| Propylene Glycol | 12.0 |
| Composite of PVP (polyvinyl pyrrolidone) | 0.83 |
| and MVE-MAHE (Methyl Vinyl Ether/ | |
| Maleic Acid Half Ester | |
| 20% aqueous dipropylene glycol solution | 0.24 |
| of 1,2-benzisothiazolin-3-one | |
| Silicone Defoamer | 0.30 |
| Sodium Salt of maleated soybean oil | 10.0 |
| Red Pigment | 2.50 |
| Water in Litres | q.s.p. 100 mL |

### Example 10: Seed Coating

100 g seed coating composition is prepared in a 250 ml glass beaker.

2.50 g of Hydrophobically modified maleated soybean oil and 7.5 g of Na Salt of maleated soybean oil in 45.56 g water, 12.00 g propylene glycol is added to beaker then combined via overhead mixer with a propellor mixing blade at 400 rpm for 10 minutes. 0.25 g Xanthan Gum added slowly and mixed for 15 minutes or until fully hydrated Composite of PVP (polyvinyl pyrrolidone) and MVE-MAHE (Methyl Vinyl Ether/Maleic Acid Half Ester Neutralized) copolymer, Silicone Defoamer and 20% aqueous dipropylene glycol solution of 1,2-benzisothiazolin-3-oneGXL added with mixing. Mixing speed increased to 600 rpm, then Zinc oxide, Zinc EDTA, and Sodium Molybdate and Red Pigment added slowly and mixed for 15 minutes.

TABLE 14
Seed Coating

| Ingredients | Wt. grams |
|---|---|
| Zinc Oxide | 25 |
| Sodium Molybdate | 0.26 |
| Zinc EDTA | 3.33 |
| Xanthan Gum | 0.25 |
| Propylene Glycol | 12 |
| Composite of PVP (polyvinyl pyrrolidone) | 0.83 |
| and MVE-MAHE (Methyl Vinyl Ether/ | |
| Maleic Acid Half Ester | |
| 20% aqueous dipropylene glycol solution | 0.24 |
| of 1,2-benzisothiazolin-3-one | |
| Silicone Defoamer | 0.3 |
| Hydrophobically modified maleated | 2.5 |
| soybean oil | |
| Na Salt of maleated soybean oil | 7.5 |
| Red Pigment | 2.5 |
| Water | q.s.p. 100 mL |

100 g seed coating composition is prepared in a 250 ml glass beaker. 10.0 g of Na Salt of hydrophobically modified maleated soybean oil in 45.56 g water, 12.00 g propylene glycol is added to beaker then combined via overhead mixer with a propellor mixing blade at 400 rpm for 10 minutes. 0.25 g Xanthan Gum added slowly and mixed for 15 minutes or until fully hydrated Composite of PVP (polyvinyl pyrrolidone) and MVE-MAHE (Methyl Vinyl Ether/Maleic Acid Half Ester Neutralized) copolymer, Silicone Defoamer and 20% aqueous dipropylene glycol solution of 1,2-benzisothiazolin-3-oneGXL added with mixing. Mixing speed increased to 600 rpm, then Zinc oxide, Zinc EDTA, and Sodium Molybdate and Red Pigment added slowly and mixed for 15 minutes.

TABLE 15
Seed Coating

| Ingredients | Wt. grams |
|---|---|
| Zinc Oxide | 25.0 |
| Sodium Molybdate | 0.26 |
| Zinc EDTA | 3.33 |
| Xanthan Gum | 0.25 |
| Propylene Glycol | 12.0 |
| Composite of PVP (polyvinyl pyrrolidone) | 0.83 |
| and MVE-MAHE (Methyl Vinyl Ether/ | |
| Maleic Acid Half Ester | |
| 20% aqueous dipropylene glycol solution | 0.24 |
| of 1,2-benzisothiazolin-3-one | |
| Silicone Defoamer | 0.30 |
| Na Salt of Maleated soybean oil | 10.0 |
| Red Pigment | 2.50 |
| Water | q.s.p. 100 mL |

### Example 11

Agrochemical oil dispersion formulation comprising the insecticidal active ingredient imidacloprid is suspended in soy methyl ester, dispersants, attapulgite, hydroxy ethylcellulose and emulsifiers, further comprising the insecticidal active ingredient bifenthrin solubilized in the oil phase.

TABLE 16
Agrochemical oil dispersion formulation

| Ingredients | Wt. grams |
|---|---|
| Imidacloprid | 270 |
| Hydrophobically modified maleated | 26.5 g |
| soybean oil | |
| Na Salt of maleated soybean oil | 79.5 g |
| Attapulgite | 67.0 |
| Hydroxy ethyl cellulose | 6.5 |
| Calcium dodecyl benzene sulfonate | 17.0 |
| Ethoxylated castor oil | 88.0 |
| EO/PO butanol | 6.0 |
| Bifenthrin | 100 |
| Soy methyl ester in Liters | q.s.p 1 L |

The process for preparing the formulation of Example 13 is performed by dispersing the imidacloprid in soy methyl ester in the presence of a mixture of NA Salt of maleated soybean oil, hydrophobically modified maleated soyabean oil and attapulgite followed by milling this mixture until the particles reach 1 to 5 μm in size. Finally, under stirring, bifenthrin, calcium dodecyl benzene sulfonate, ethoxylated castor oil, EO/PO butanol, hydroxy ethylcellulose and, once again, attapulgite are added.

### Example 12

Agrochemical oil dispersion formulation comprising the insecticidal active ingredient imidacloprid is suspended in soy methyl ester, dispersants, attapulgite, hydroxy ethylcellulose and emulsifiers, further comprising the insecticidal active ingredient bifenthrin solubilized in the oil phase.

TABLE 17
Agrochemical oil dispersion

| Ingredients | Wt. grams |
|---|---|
| Imidacloprid | 270 |
| Na Salt of hydrophobically modified | 106 |
| Maleated soybean oil | |
| Attapulgite | 67.0 |
| Hydroxy ethyl cellulose | 6.5 |
| Calcium dodecyl benzene sulfonate | 17.0 |
| Ethoxylated castor oil | 88.0 |
| EO/PO butanol | 6.0 |
| Bifenthrin | 100 |
| Soy methyl ester in Liters | q.s.p 1 L |

The process for preparing the formulation of Example 14 is performed by dispersing the imidacloprid in soy methyl ester in the presence of Na salt of hydrophobically maleated soybean oil and attapulgite followed by milling this mixture until the particles reach 1 to 5 μm in size. Finally, under stirring, bifenthrin, calcium dodecyl benzene sulfonate, ethoxylated castor oil, EO/PO butanol, hydroxy ethylcellulose and, once again, attapulgite are added.

### Example 13—Herbicides

A sodium salt of hydrophobically modified maleated soybean oil could be used to disperse herbicides in an oil-based solution, allowing for better and more consistent coverage of weeds in agricultural fields. This could improve the efficacy of the herbicide and reduce the amount of herbicide required.

For example, a formulation for an oil-based herbicide dispersion could include 20-50% by weight of the herbicide active ingredient, 5-50% by weight of an oil carrier such as mineral oil or vegetable oil, and 1-5% by weight of the non-aqueous dispersant. The specific concentrations will depend on the properties of the herbicide and oil, as well as the target application.

TABLE 18
Herbicide composition

| Ingredients | Wt. % |
|---|---|
| Halosulfuron-methyl | 20-50 |
| Mineral Oil | 5-50 |
| Na Salt of Hydrophobically modified | 1-5 |
| maleated soybean oil | |

### Example 14—Insecticides

A sodium salt of hydrophobically modified maleated soybean oil could also be used to disperse insecticides in oil-based solutions. This could be particularly useful in greenhouse applications, where water-based sprays may be less effective or more prone to causing damage to plants.

For example, a formulation for an oil-based insecticide dispersion could include 1-10% by weight of the insecticide active ingredient, 90-99% by weight of an oil carrier such as canola oil or soybean oil, and 1-5% by weight of the non-aqueous dispersant. The specific concentrations will depend on the properties of the insecticide and oil, as well as the target application.

TABLE 19
Insecticide Composition

| Ingredients | Wt. % |
|---|---|
| Imidacloprid | 1-10 |
| Canola oil | 90-99 |
| Na Salt of Hydrophobically modified | 1-5 |
| maleated soybean oil | |

### Example 15—Fungicides

A sodium salt of hydrophobically modified maleated soybean oil could also be used to disperse fungicides in oil-based solutions, which could be particularly useful for treating fungal infections on crops or in soil.

For example, a formulation for an oil-based fungicide dispersion could include 10-30% by weight of the fungicide active ingredient, 70-90% by weight of an oil carrier such as vegetable oil or mineral oil, and 1-5% by weight of the non-aqueous dispersant. The specific concentrations will depend on the properties of the fungicide and oil, as well as the target application.

TABLE 20
Fungicide Composition

| Ingredients | Wt. % |
|---|---|
| Mycobutanil | 10-30 |
| Soybean Oil | 70-90 |
| Na Salt of Hydrophobically modified | 1-5 |
| maleated soybean oil | |

### Example 16—Seed Coatings

A sodium salt of hydrophobically modified maleated soybean oil could also be used as a component of seed coatings, allowing for better adhesion and coverage of pesticides on the seed surface. This could improve the efficiency of the pesticides and reduce the amount of pesticide required per seed.

For example, a formulation for a seed coating could include 1-20% by weight of the pesticide, 5-30% by weight of a binder such as starch or gum, 50-90% by weight of an oil carrier such as vegetable oil or mineral oil, and 1-5% by weight of the non-aqueous dispersant. The specific concentrations will depend on the properties of the pesticide, binder, oil, and dispersant, as well as the target seed and application.

TABLE 21
Seed coating Pesticide Composition

| Ingredients | Wt. % |
|---|---|
| Thiamethoxam | 1-20 |
| Gum arabic | 5-30 |
| Vegetable Oil | 50-90 |
| Na Salt of Hydrophobically modified | 1-5 |
| maleated soybean oil | |

### Example 17—Livestock Applications

A sodium salt of hydrophobically modified maleated soybean oil could also be used in livestock applications, such as to disperse insecticides for treating pests on livestock or to disperse medications in oil-based solutions for treating livestock illnesses.

For example, a formulation for an oil-based insecticide for livestock could include 1-10% by weight of the insecticide active ingredient, 90-99% by weight of an oil carrier such as mineral oil or vegetable oil, and 1-5% by weight of the non-aqueous dispersant. The specific concentrations will depend on the properties of the insecticide and oil, as well as the target application and type of livestock.

TABLE 22
Livestock Composition

| Ingredients | Wt. % |
|---|---|
| Cypermethrin | 1-10 |
| Mineral Oil | 90-99 |
| Na Salt of Hydrophobically modified | 1-5 |
| maleated soybean oil | |

### Example 18

A sodium salt of hydrophobically modified maleated soybean oil could be used in seed coatings to help adhere active ingredients to seeds, ensuring that the pesticides or other treatments are delivered directly to the plant during germination. The non-aqueous binder could be mixed with the active ingredients and any other necessary additives, then applied to the seed using a seed treater or other appropriate equipment.

TABLE 23
Seed coatings

| Ingredients | Wt. % |
|---|---|
| Thiamethoxam | 1-20 |
| Polyvinyl alcohol | 1-10 |
| Na Salt of Hydrophobically modified | 1-10 |
| maleated soybean oil | |
| Talc | 10-60 |

### Example 19

Granular formulations are commonly used in agriculture to apply pesticides or other treatments to soil. A sodium salt of hydrophobically modified maleated soybean oil be used to bind the active ingredient and other necessary additives into a granular form, which could be applied using a broadcast spreader or other appropriate equipment.

TABLE 24
Granular composition

| Ingredients | Wt. % |
|---|---|
| Imidacloprid | 10-50 |
| Clay | 10-60 |
| Na Salt of Hydrophobically modified | 1-10 |
| maleated soybean oil | |
| alkylphenol ethoxylate | 1-5 |

### Example 20

A sodium salt of hydrophobically modified maleated soybean oil could be used in controlled-release formulations to ensure that active ingredients are released over a specific period of time, providing long-lasting protection to crops. The non-aqueous binder could be used to coat the active ingredients and any necessary carriers, then applied to the soil or directly to the plant using appropriate equipment.

TABLE 25
Controlled release composition

| Ingredients | Wt. % |
|---|---|
| Chlorpyrifos | 10-50 |
| Clay | 1-10 |
| Na Salt of Hydrophobically modified | 1-10 |
| maleated soybean oil | |
| Hydroxypropyl methylcellulose | 1-10 |

### Example 21

A sodium salt of hydrophobically modified maleated soybean oil could be used in film coatings to provide a protective barrier around seeds or other plant material. The non-aqueous binder could be used to coat the surface of the seed or plant material, providing protection against pests and diseases while allowing the seed to germinate or the plant to grow.

TABLE 26
Film coating composition

| Ingredients | Wt. % |
|---|---|
| Tebuconazole | 10-40 |
| Calcium carbonate | 10-50 |
| Na Salt of Hydrophobically modified | 1-10 |
| maleated soybean oil | |
| Magnesium stearate | 1-10 |

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed and/or claimed inventive concept(s).

We claim:

1. An agricultural composition comprising a reaction product of
(a) a maleated natural oil, comprising a natural oil with maleated functionality; and
(b) a base.

2. The agricultural composition according to claim 1, wherein the reaction product comprises a maleated functionality fully or partially reacted with the base.

3. The agricultural composition according to claim 1, wherein the base is selected from the group consisting of inorganic bases, organic bases, and mixtures thereof.

4. The agricultural composition according to claim 3, wherein the inorganic base is selected from the group consisting of oxides of alkali metals and alkaline earth metals, hydroxides of alkali metals and alkaline earth metals, carbonates of alkali metals and alkaline earth metals, bicarbonates of alkali metals and alkaline earth metals, oxides of transition metals, hydroxides of transition metals, carbonates of transition metals, bicarbonates of transition metals, and combinations thereof.

5. The agricultural composition according to claim 3, wherein the organic base is selected from the group consisting of ammonia, primary amines, secondary amines, pyridine, imidazole, benzimidazole, histidine, guanidine, and mixtures thereof.

6. The agricultural composition according to claim 4, wherein the alkali metal is selected from the group consisting of lithium, sodium, potassium, rubidium, cesium, francium, and mixtures thereof.

7. The agricultural composition according to claim 4, wherein the alkaline earth metal is selected from the group consisting of beryllium, magnesium, calcium, strontium, barium, radium, and mixtures thereof.

8. The agricultural composition according to claim 4, wherein the transition metal is selected from the group consisting of scandium, titanium, vanadium, chromium, manganese, iron, cobalt, nickel, copper, zinc, yttrium zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, darmstadtium, roentgenium, and mixtures thereof.

9. The agricultural composition according to claim 4, wherein the inorganic base is selected from the group consisting of oxides, hydroxides, carbonates and bicarbonates of sodium, calcium, and combinations thereof.

10. The agricultural composition according to claim 1, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, maleated chickpea oils, maleated clary sage oils, and mixture thereof.

11. The agricultural composition according to claim 1, wherein the maleated natural oil is a maleated soybean oil.

12. The agricultural composition according to claim 1, wherein the reaction product comprises one or more structure selected from the group of structures, comprising maleated functionality fully or partially reacted with the base, consisting of the following structures: 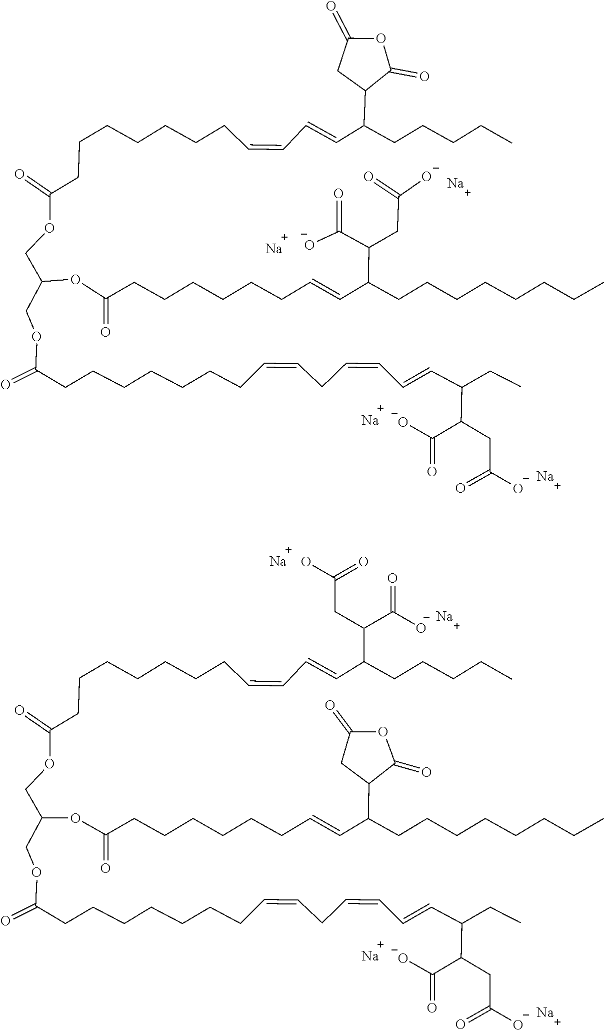 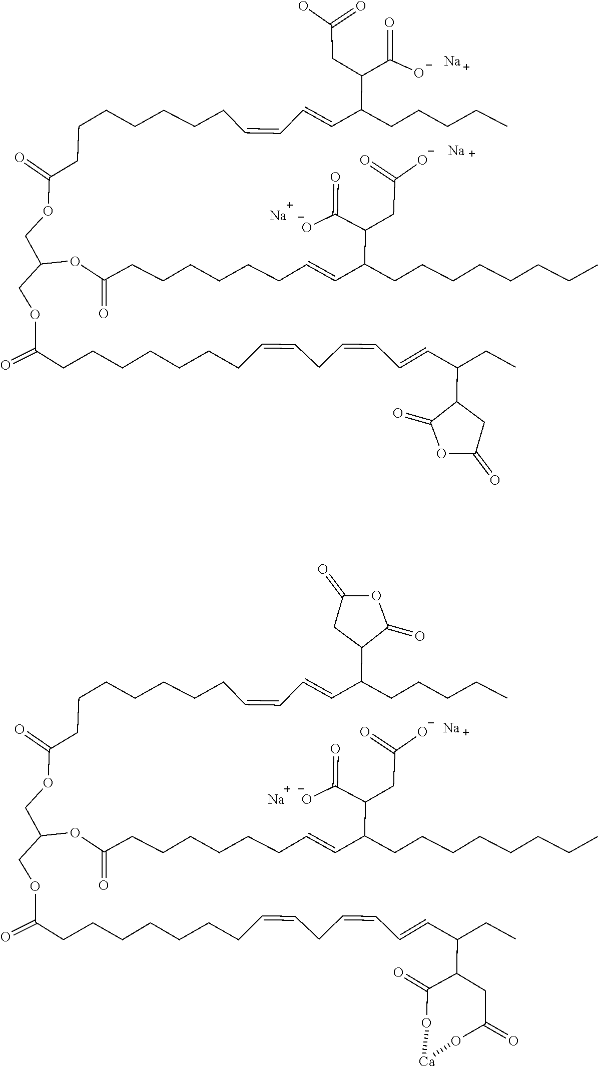 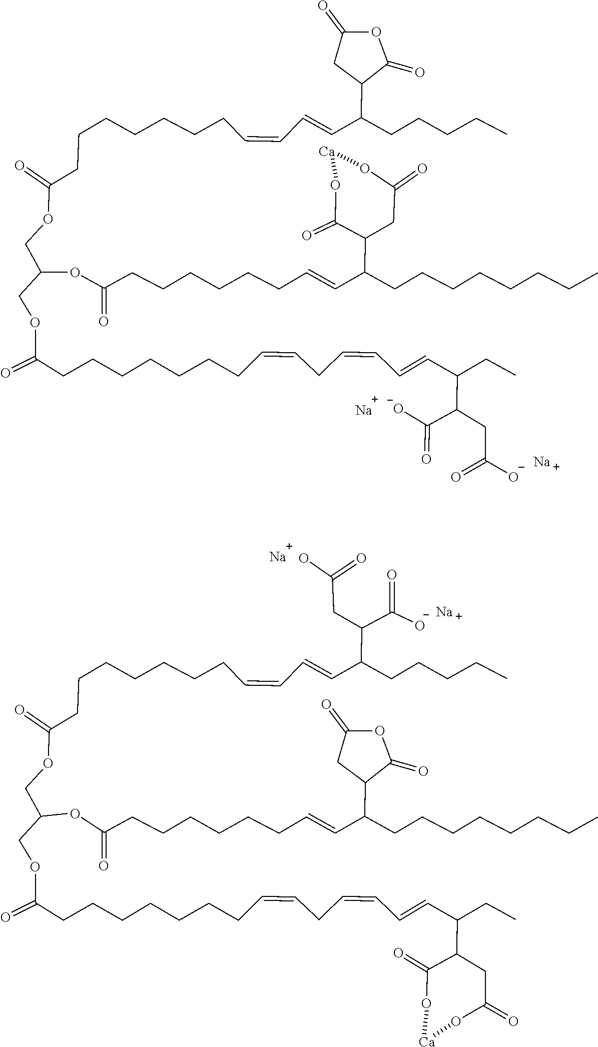 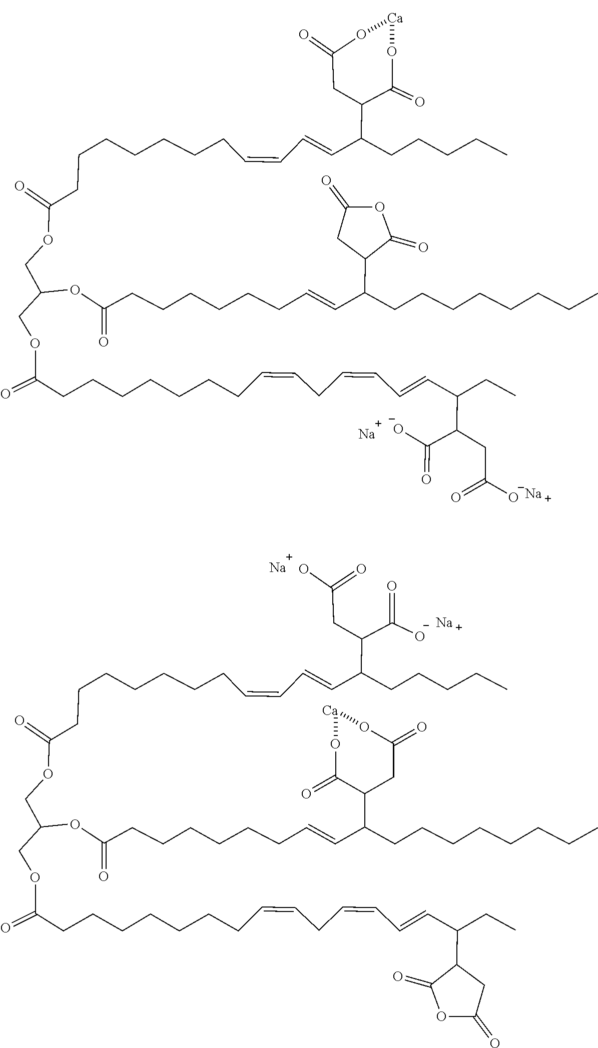 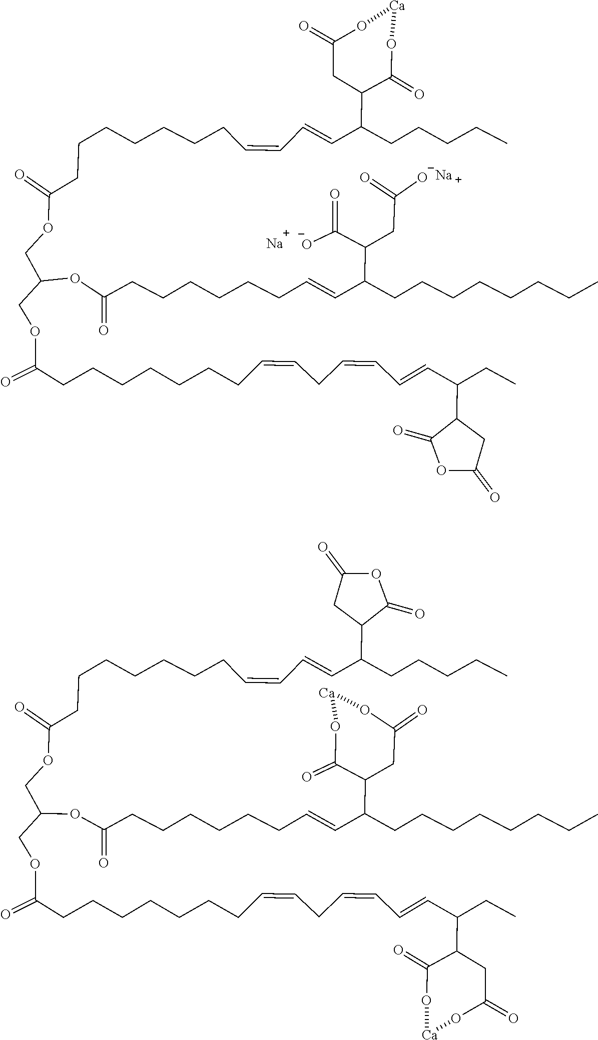 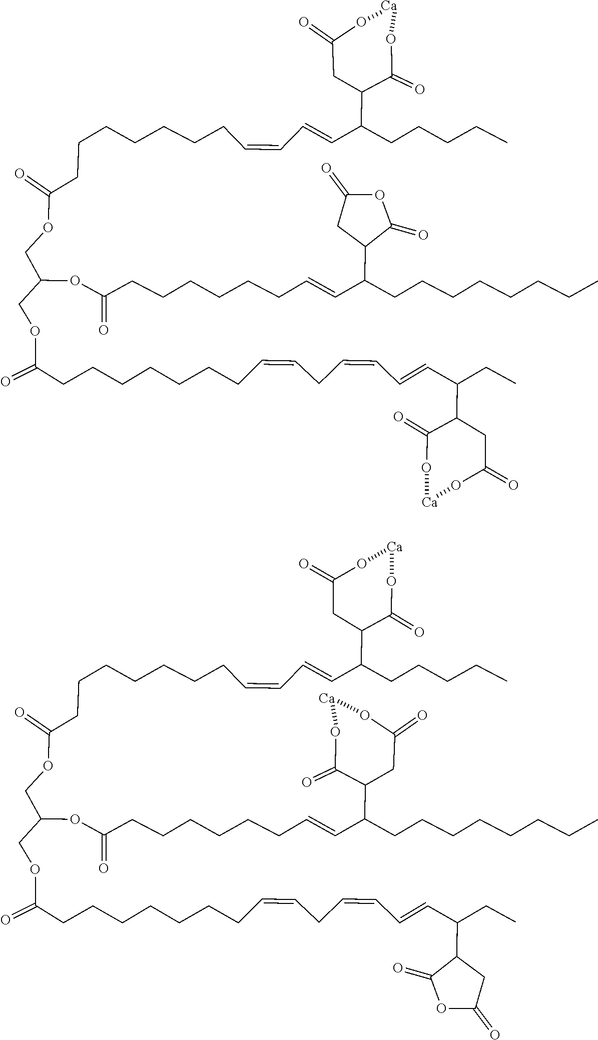 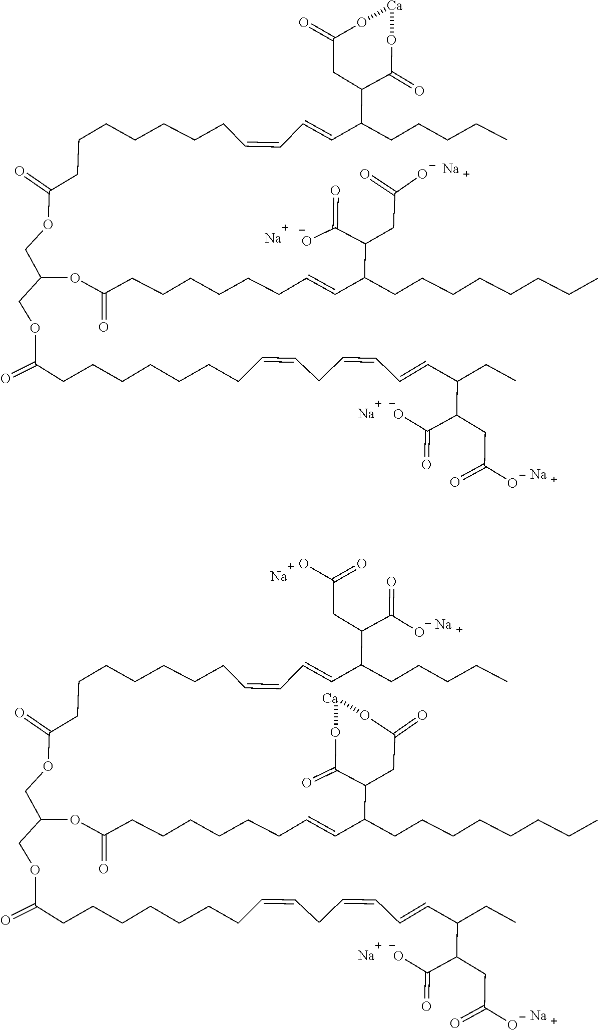 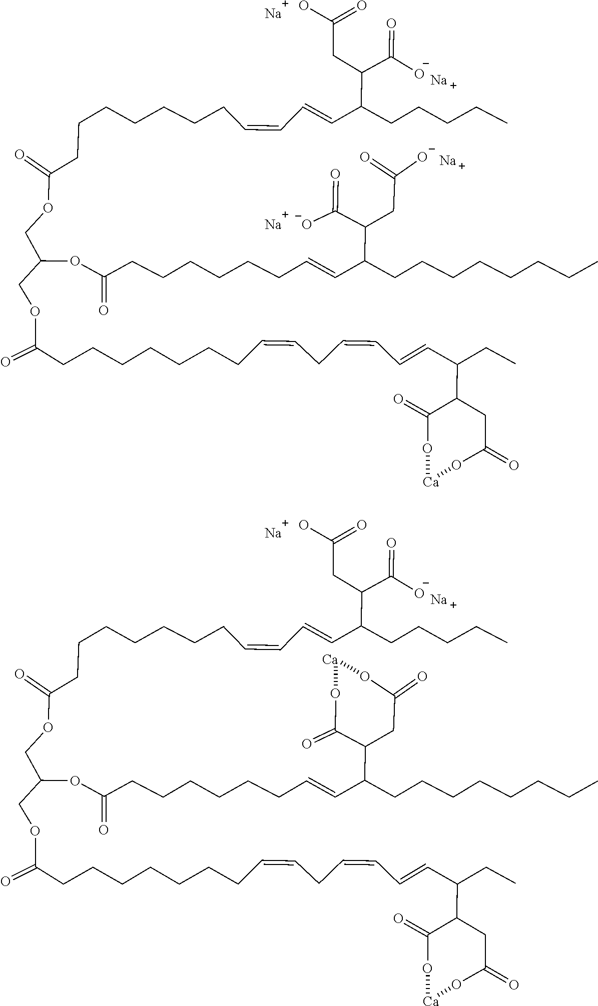 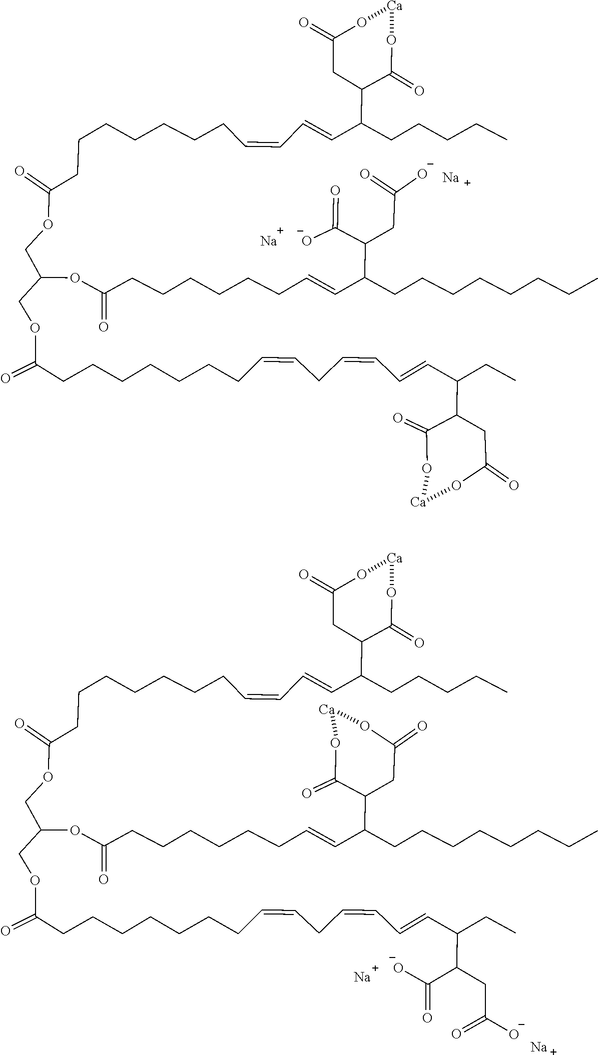 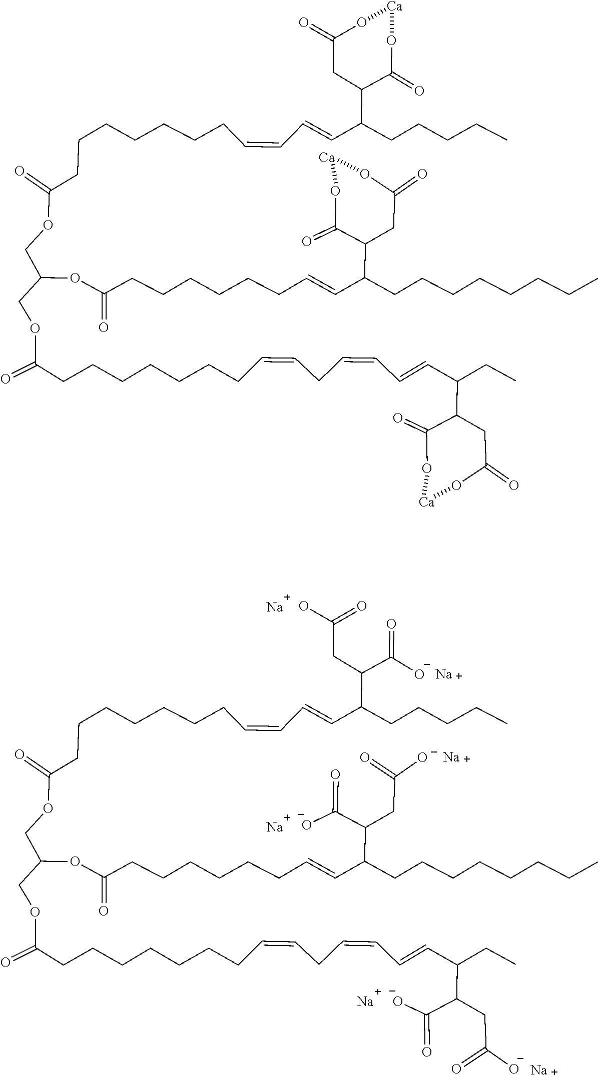
and combinations thereof.

13. The agricultural composition according to claim 1, wherein the agricultural composition further comprises one or more agriculturally active ingredient and one or more additional ingredient.

14. The agricultural composition according to claim 13, wherein the agriculturally active ingredient is selected from the group consisting of fertilizers and pesticides selected from the group consisting of rodenticides, miticides, algicides, molluscicides, acaricides, avicides, insecticides, herbicides, ovicides, fungicides, germicides, antibiotics, antibacterials, antivirals, antifungals, antiprotozoals, anti-parasites and antimicrobials.

15. The agricultural composition according to claim 13, wherein the additional ingredient is an adjuvant or an inert ingredient.

16. The agricultural composition according to claim 15, wherein the adjuvant is selected from the group consisting of acidifying agents, buffering agents, anti-foam agents, defoaming agents, anti-transpirants, dyes and brighteners, compatibility agents, crop oil concentrates, oil surfactants, deposition agents, drift reduction agents, foam markers, feeding stimulants, herbicide safeners, spreaders, extenders, adhesive agents, suspension agents, gelling agents, synergists, wetting agents, emulsifiers, dispersing agents, penetrants, tank and equipment cleaners, neutralizers, water absorbents, water softeners and mixtures thereof.

17. The agricultural composition according to claim 13, wherein the additional ingredient is selected from the group consisting of solvents, liquid carriers, solid carriers or fillers, surfactants, solubilizers, penetration enhancers, protective colloids, thickeners, humectants, repellents, attractants, compatibilizers, anti-freezing agents, crystallization inhibitors, colorants, tackifiers, binders, preservatives, pH adjuster, clarifiers, stabilizers, UV stabilizers, and mixtures thereof.

18. The agricultural composition according to claim 1, where in the agricultural composition is an adjuvant composition, a fertilizer composition, a nutrient composition, a plant strengthener composition, a seed coating composition, a soil conditioner composition, a livestock composition, a granular composition, a controlled release composition, a film coating composition, a pesticide composition selected from the group consisting of ovicide, rodenticide, insecticide, miticide, algicide, molluscicide, acaricide, avicide, fungicide and herbicide compositions, a germicide composition, an antibiotic composition, an antibacterial composition, an antiviral composition, an antifungal composition, an antiprotozoal composition, an anti-parasite composition, a wood preservation composition, an antimicrobial composition or a yield enhancer composition to soil for the plant.

19. The agricultural composition according to claim 1, wherein the agricultural composition is in the form of an aqueous or a non-aqueous composition comprising a capsule suspension, an emulsifiable concentrate, an emulsion for seed treatment, a concentrated aqueous emulsion, a microemulsion, a suspoemulsion, an oil-in-water emulsion, a flowable concentrate for seed treatment, an oil dispersion, a suspension concentrate, a water dispersible granule or a wettable powder.

20. The agricultural composition according to claim 1, wherein the agricultural composition is formulated as a liquid, a gel, a paste, a powder, a granule, a tablet, an emulsion, or a pellet.

21. The agricultural composition according to claim 1, wherein the agricultural composition is for topical application to an animal or a plant and comprises a safe and effective amount of
(A) from about 0.01 to about 20.0 wt. % of a reaction product of (a) a maleated natural oil, comprising a natural oil with maleated functionality; and (b) a base; and
(B) from about 0.1 to about 90.0 wt. % of one or more agriculturally active ingredient; and
(C) from about 1.0 to about 99.0 wt. % of one or more additional ingredient.

22. The agricultural composition according to claim 1, wherein the agricultural composition comprises:
(A) from about 0.01 to about 20.0 wt. % of a reaction product of (a) a maleated soybean natural oil, comprising a soybean oil with maleated functionality; and (b) a base selected from the group consisting of sodium hydroxide, potassium hydroxide, calcium hydroxide; and mixtures thereof; and
(B) from about 0.1 to about 90.0 wt. % of one or more agriculturally active ingredient; and
(C) from about 1.0 to about 99.0 wt. % of one or more additional ingredient.

23. A method for treating vegetation comprising applying an agricultural composition of claim 1 to a plant, plant foliage, blossoms, leaves, stems, fruits, the area adjacent to a plant, soil, seeds, germinating seeds, roots, liquid and solid growth media, flower, bud, or hydroponic growth solutions.

24. A method of treating an animal comprising topically applying an agricultural composition of claim 1 to an area of skin of an animal.

25. An agricultural composition comprising a reaction product of:
(A) a maleated natural oil, comprising a natural oil with maleated functionality;
(B) a base; wherein the maleated functionality has been partially reacted with the base; and
(C) a functionalized or unfunctionalized moiety selected from the group consisting of hydrophobic moieties, hydrophilic moieties, and combinations thereof; wherein the maleated functionality has been partially reacted with the functionalized or unfunctionalized moiety.

26. The agricultural composition according to claim 25, wherein the hydrophobic moiety is a moiety selected from those of the group consisting of unsubstituted or substituted alkyl, cycloalkyl, alkenyl, and aryl alcohols and amines, wherein the alcohols and amines contain from about 6 to about 36 carbon atoms and may contain heteroatoms; unsubstituted or substituted polyols, with or without heteroatoms, containing about 37 to about 60 carbon atoms; silicon-based compounds; and combinations thereof.

27. The agricultural composition according to claim 25, wherein the hydrophilic moiety is a moiety selected from those of the group consisting of unsubstituted or substituted alkyl, cycloalkyl, alkenyl, and aryl alcohols and amines, wherein the alcohols and amines contain from about 1 to about 5 carbon atoms and may contain heteroatoms; unsubstituted or substituted polyols, wherein the unsubstituted or substituted polyols contain from about 2 to about 36 carbon atoms and may contain heteroatoms; poly(ethylene glycol) monomethyl ethers (mPEGs) containing from 5 to 45 carbon atoms; silanes; and combinations thereof.

28. The agricultural composition according to claim 27, wherein the silane is functionalized with an alcohol, an amine, or combinations thereof.

29. The agricultural composition according to claim 26, wherein the hydrophobic moiety is that of an alcohol selected from the group consisting of hexanol, heptanol, nonanol, decanol, dodecanol, phenol, ethylbenzyl alcohol, 2-ethyl-1-hexanol, 1-octanol, 2-octanol, 2-butyl-1-octanol, 2-hexyl-1-decanol, 2-octyl-1-dodecyl alcohol, 2-tetradecanol, 2-hexadecanol, 3,7-dimethyl-1-octanol, 2-propyl-1-pentanol, 4-methyl-1-pentanol, and mixtures thereof.

30. The agricultural composition according to claim 27, wherein the hydrophilic moiety is that of an alcohol selected from the group consisting of methanol, ethanol, propanol, isopropanol, butanol, and mixtures thereof.

31. The agricultural composition according to claim 27, wherein the hydrophilic moiety is that of a polyol selected from the group consisting of ethylene glycol, propylene glycol, diethylene glycol, dipropylene glycol, dibutylene glycol, polyethylene glycol, polypropylene glycol, hexylene glycol, sorbitol, neopentylglycol, eythritol, mannitol, xylitol, threitol, pentaerythritol, beta-cyclodextrin, ribose, 2-deoxy-D-galactose, D-glucosamine, D-mannosamine, D-galactosamine, N-methyl-D-glucosamine, and mixtures thereof.

32. The agricultural composition according to claim 26, wherein the hydrophobic moiety is that of an amine selected from the group consisting of benzylamine, cyclohexylamine, hexylamine, methylhexylamine, phenethylamine, octylamine, oleylamine, decylamine, dodecylamine, hexadecylamine, octadecylamine, undecylamine, pentadecylamine, and mixtures thereof.

33. The agricultural composition according to claim 27, wherein the hydrophilic moiety is that of an amine selected from the group consisting of 2-methylpentane-1,5-diamine, diethanolamine, serinol, 2-amino-2-ethyl-1,3-propanediol, dimethylamine, 2-methylbutylamine, 3-amino-1-propanol, their hydrochloride salts, their ammonium salts, and mixtures thereof.

34. The agricultural composition according to claim 26, wherein the silicon based compound is a hydrophobic compound selected from the group consisting of aminopropylmethylsiloxane-dimethylsiloxane, N-ethylaminoisobutyl terminated polydimethylsiloxane, poly(1,1-dimethylsilazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine]terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

35. The agricultural composition according to claim 27, wherein the silane is a hydrophilic compound selected from the group consisting of 3-aminopropylsilanetriol, N-(2-aminoethyl)-3-aminopropylsilanetriol, and mixtures thereof.

36. The agricultural composition according to claim 25, wherein the base is selected from the group consisting of inorganic bases, organic bases, and mixtures thereof.

37. The agricultural composition according to claim 36, wherein the inorganic base is selected from the group consisting of oxides of alkali metals and alkaline earth metals, hydroxides of alkali metals and alkaline earth metals, carbonates of alkali metals and alkaline earth metals, bicarbonates of alkali metals and alkaline earth metals, oxides of transition metals, hydroxides of transition metals, carbonates of transition metals, bicarbonates of transition metals, and combinations thereof.

38. The agricultural composition according to claim 36, wherein the organic base is selected from the group consisting of ammonia, primary amines, secondary amines, pyridine, imidazole, benzimidazole, histidine, guanidine, and mixtures thereof.

39. The agricultural composition according to claim 37, wherein the alkali metal is selected from the group consisting of lithium, sodium, potassium, rubidium, cesium, francium, and mixtures thereof.

40. The agricultural composition according to claim 37, wherein the alkaline earth metal is selected from the group consisting of beryllium, magnesium, calcium, strontium, barium, radium, and mixtures thereof.

41. The agricultural composition according to claim 37, wherein the transition metal is selected from the group consisting of scandium, titanium, vanadium, chromium, manganese, iron, cobalt, nickel, copper, zinc, yttrium zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, darmstadtium, roentgenium, and mixtures thereof.

42. The agricultural composition according to claim 37, wherein the inorganic base is selected from the group consisting of oxides, hydroxides, carbonates and bicarbonates of sodium, calcium, and combinations thereof.

43. The agricultural composition according to claim 25, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, maleated chickpea oils, maleated clary sage oils, and mixtures thereof.

44. The agricultural composition according to claim 25, wherein the maleated natural oil is a maleated soybean oil.

45. The agricultural composition according to claim 25, wherein the reaction product comprises one or more structure selected from the following group of structures: 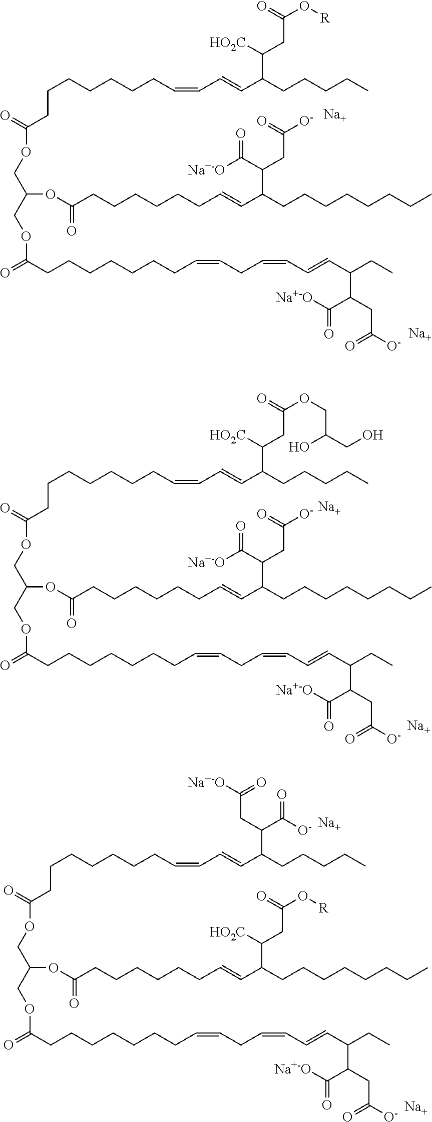 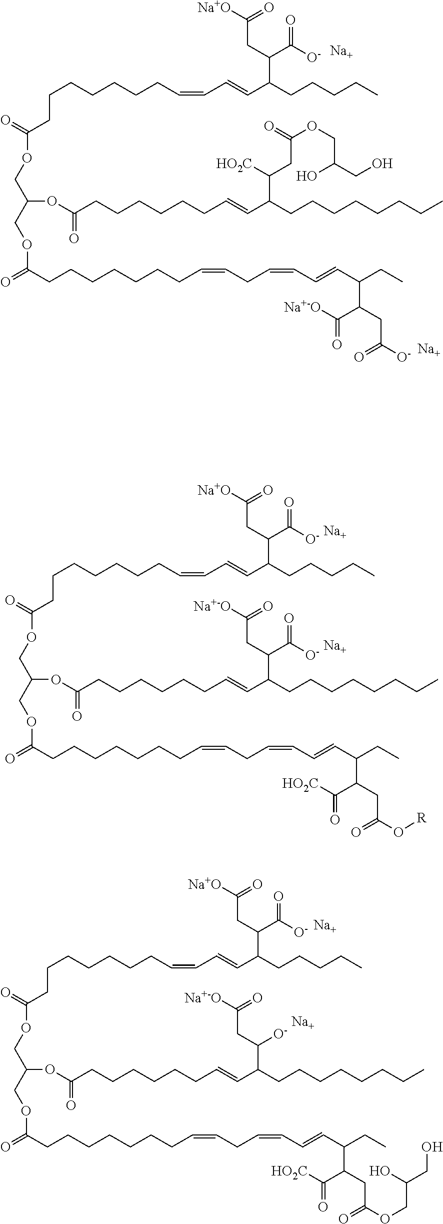 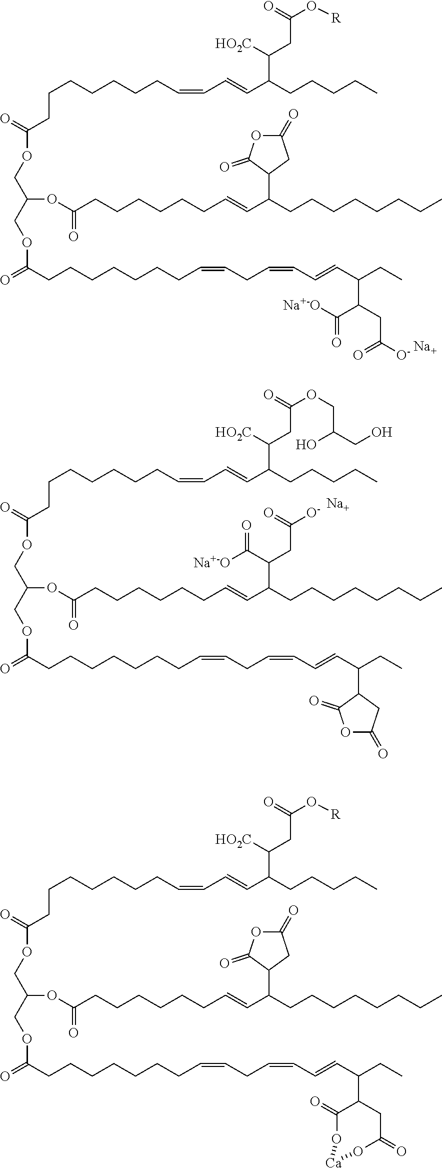 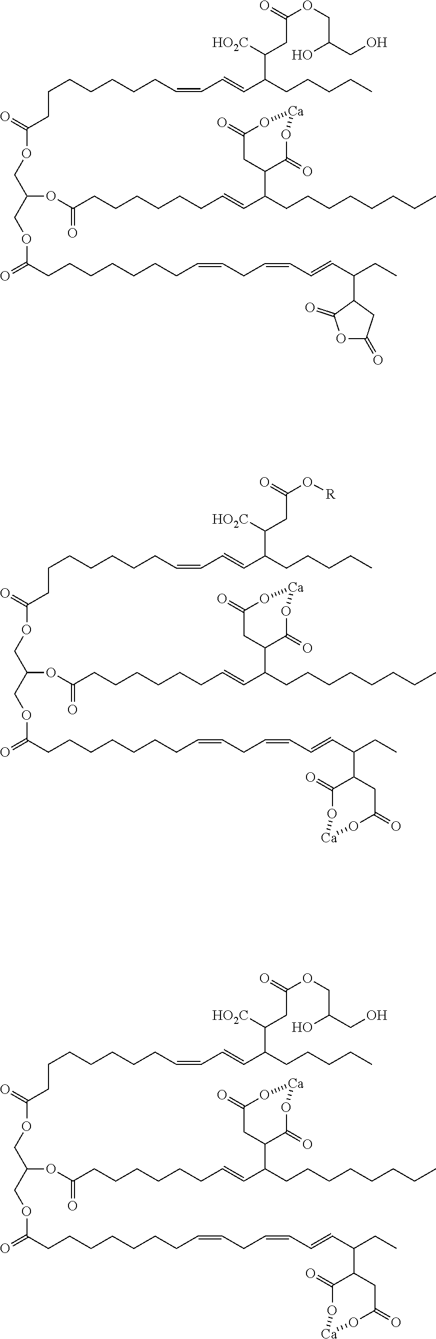 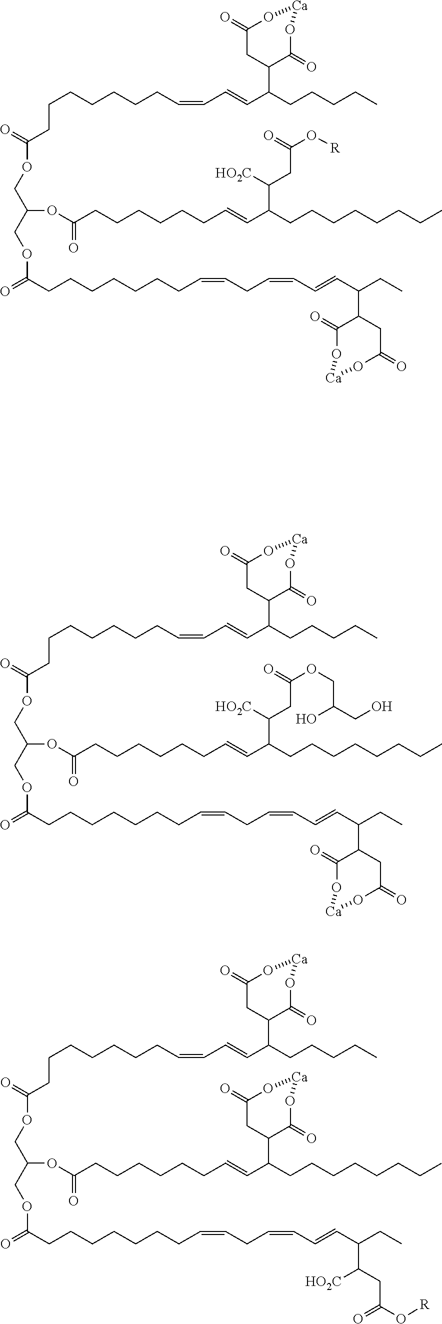 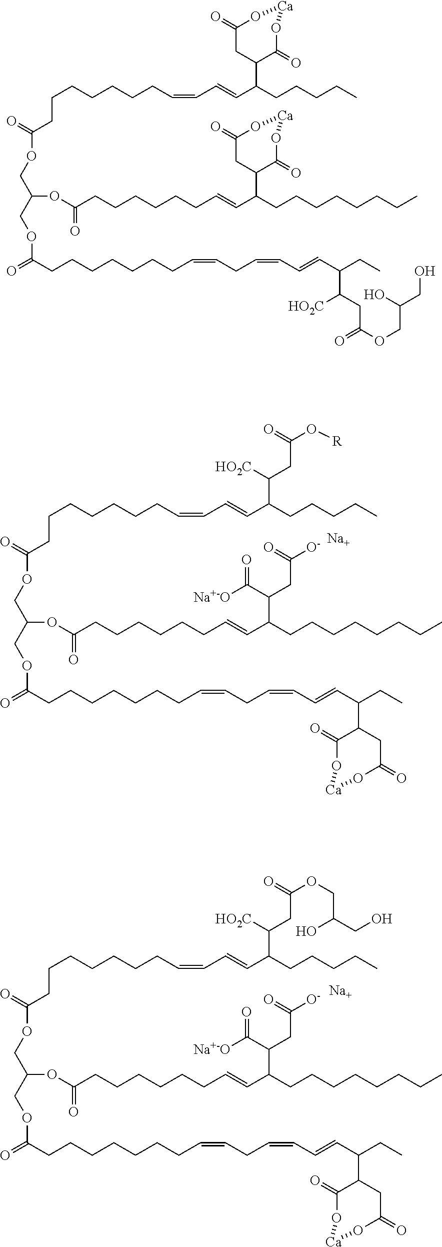 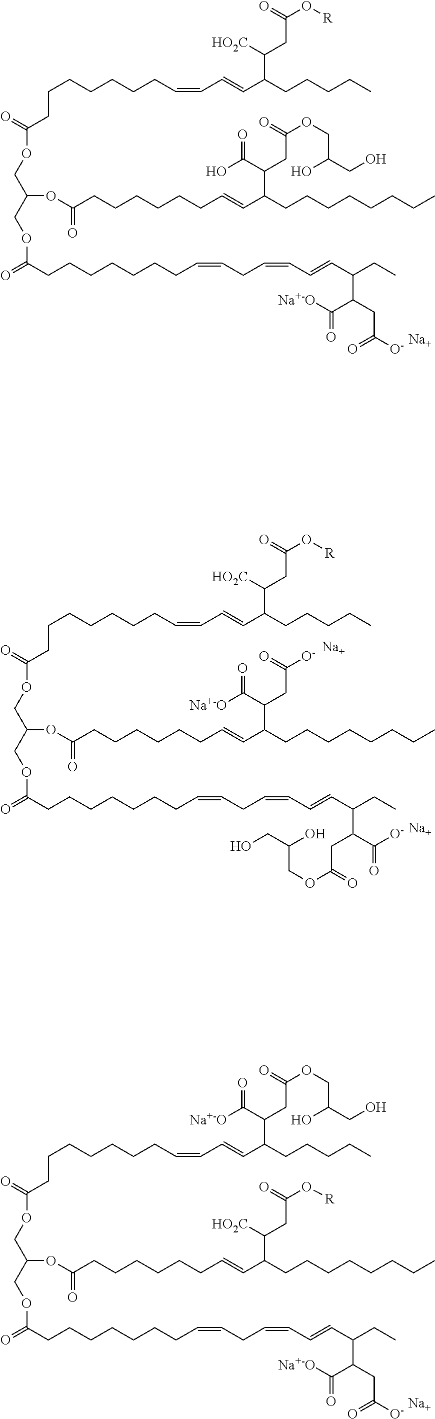 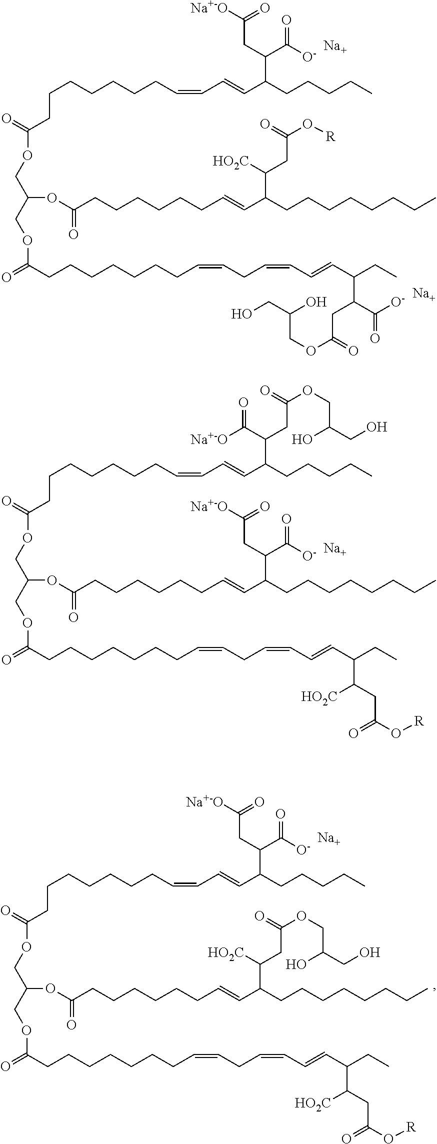
and combinations thereof, wherein R is ethyl, butyl, hexyl, octyl, 2-ethylhex-1-yl, 2-butyloct-1-yl, 2-hexyldec-1-yl, 2-octyldodec-1-yl or mixtures thereof.

46. The agricultural composition according to claim 25, wherein the agricultural composition further comprises one or more agriculturally active ingredient and one or more additional ingredient.

47. The agricultural composition according to claim 46, wherein the agriculturally active ingredient is selected from the group consisting of fertilizers and pesticides selected from the group consisting of rodenticides, miticides, algicides, molluscicides, acaricides, avicides, insecticides, herbicides, ovicides, fungicides, germicides, antibiotics, antibacterials, antivirals, antifungals, antiprotozoals, anti-parasites and antimicrobials.

48. The agricultural composition according to claim 46, wherein the additional ingredient is an adjuvant or an inert ingredient.

49. The agricultural composition according to claim 48, wherein the adjuvant is selected from the group consisting of acidifying agents, buffering agents, anti-foam agents, defoaming agents, anti-transpirants, dyes and brighteners, compatibility agents, crop oil concentrates, oil surfactants, deposition agents, drift reduction agents, foam markers, feeding stimulants, herbicide safeners, spreaders, extenders, adhesive agents, suspension agents, gelling agents, synergists, wetting agents, emulsifiers, dispersing agents, penetrants, tank and equipment cleaners, neutralizers, water absorbents, water softeners and mixtures thereof.

50. The agricultural composition according to claim 48, wherein the additional ingredient is selected from the group consisting of solvents, liquid carriers, solid carriers or fillers, surfactants, solubilizers, penetration enhancers, protective colloids, thickeners, humectants, repellents, attractants, compatibilizers, bactericides, anti-freezing agents, crystallization inhibitors, colorants, tackifiers, binders, preservatives, pH adjuster, clarifiers, stabilizers, UV stabilizers, and mixtures thereof.

51. The agricultural composition according to claim 25, where in the agricultural composition is an adjuvant composition, a fertilizer composition, a nutrient composition, a plant strengthener composition, a seed coating composition, a soil conditioner composition, a livestock composition, a granular composition, a controlled release composition, a film coating composition, a pesticide composition selected from the group consisting of ovicide, rodenticide, insecticide, miticide, algicide, molluscicide, acaricide, avicide, fungicide and herbicide compositions, a germicide composition, an antibiotic composition, an antibacterial composition, an antiviral composition, an antifungal composition, an antiprotozoal composition, an anti-parasite composition, a wood preservation composition, an antimicrobial composition or a yield enhancer composition to soil for the plant.

52. The agricultural composition according to claim 25, wherein the agricultural composition is in the form of an aqueous or a non-aqueous composition comprising a capsule suspension, an emulsifiable concentrate, an emulsion for seed treatment, a concentrated aqueous emulsion, a microemulsion, a suspoemulsion, an oil-in-water emulsion, a flowable concentrate for seed treatment, an oil dispersion, a suspension concentrate, a water dispersible granule or a wettable powder.

53. The agricultural composition according to claim 25, wherein the agricultural composition is formulated as a liquid, a gel, a paste, a powder, a granule, a tablet, an emulsion, or a pellet.

54. An agricultural composition according to claim 25, wherein the agricultural composition is for topical application to an animal or a plant and comprises a safe and effective amount of
(A) from about 0.01 to about 20.0 wt. % of a reaction product of
(a) a maleated natural oil; and
(b) a base; wherein the maleated functionality has been partially reacted with the base; and
(c) a functionalized or unfunctionalized moiety selected from the group consisting of hydrophobic moieties, hydrophilic moieties, and combinations thereof wherein the maleated functionality has been partially reacted with the functionalized or unfunctionalized moiety;
(B) from about 1.0 to about 90.0 wt. % of one or more agriculturally active ingredient; and
(C) from about 1.0 to about 99.0 wt. % of one or more additional ingredient.

55. An agricultural composition according to claim 25, wherein the agricultural composition comprises
(A) from about 0.01 to about 20.0 wt. % of a reaction product of
(a) a maleated natural oil, comprising a natural oil with maleated functionality;
(b) a base selected from the group consisting of sodium hydroxide, potassium hydroxide, calcium hydroxide and mixtures thereof; wherein the maleated functionality has been partially reacted with the base; and
(c) a functionalized or unfunctionalized moiety which is that of an alcohol or polyol selected from the group consisting of ethanol, butanol, hexanol, octanol, 2-ethyl-1-hexanol, 2-butyl-1-octanol, 2-hexyl-1-decanol, 2-octyl-1-dodecyl alcohol, glycerol, castor oil and combinations thereof; wherein the maleated functionality has been partially reacted with the functionalized or unfunctionalized moiety;
(B) from about 1.0 to about 90.0 wt. % of one or more agricultural active ingredient; and
(C) from about 1.0 to about 99.0% of one or more additional ingredient.

56. A method for treating vegetation comprising applying an agricultural composition of claim 25 to a plant, plant foliage, blossoms, leaves, stems, fruits, the area adjacent to a plant, soil, seeds, germinating seeds, roots, liquid and solid growth media, flower, bud, or hydroponic growth solutions.

57. A method of treating an animal comprising topically applying an agricultural composition of claim 25 to an area of skin of an animal.

58. A composition comprising a reaction product of:
(A) a maleated natural oil, comprising a natural oil with maleated functionality; and
(B) a functionalized or unfunctionalized moiety selected from the group consisting of hydrophobic moieties, hydrophilic moieties, and combinations thereof, wherein the maleated functionality has been partially reacted with the functionalized or unfunctionalized moiety; and
(C) a base.

59. The composition according to claim 58, wherein the reaction product comprises a compound of the following structure: 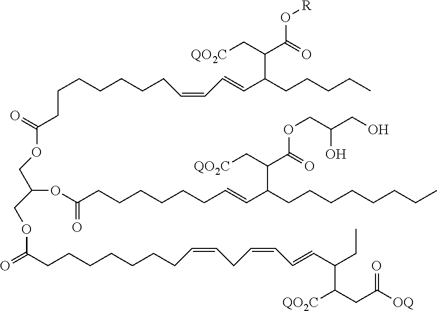
wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and
wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

60. The composition according to claim 58, wherein the reaction product is a polymer comprising the following structure: 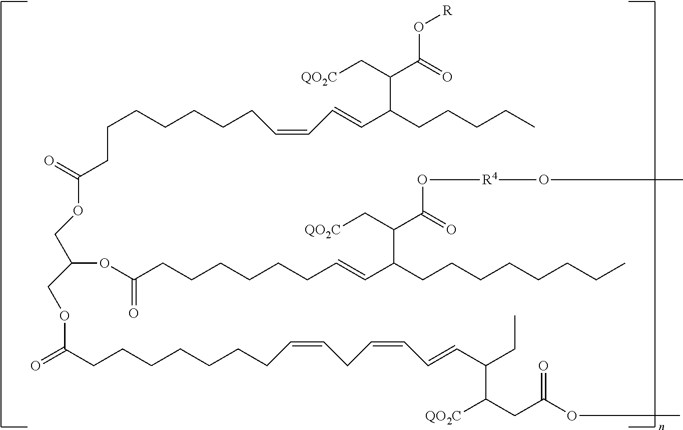
wherein n is greater than 1;
wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and
wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

61. The composition according to claim 59, wherein the reaction product comprises one or more structure selected from the group of the following structures: 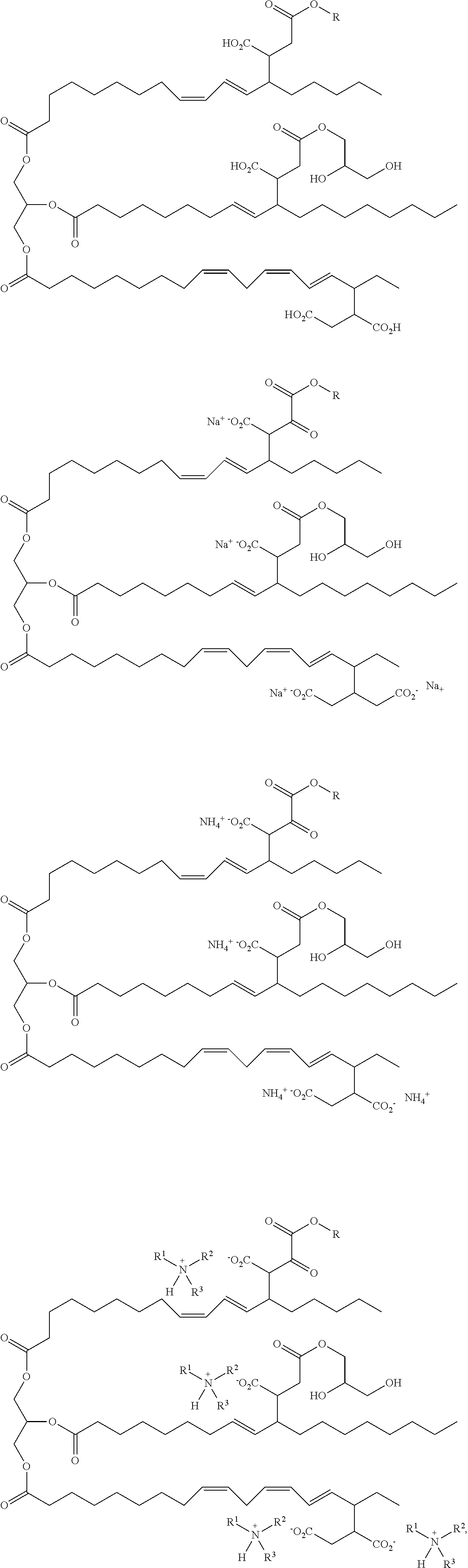
wherein R is ethyl, butyl, hexyl, octyl, 2-ethylhex-1-yl, 2-butyloct-1-yl, 2-hyxyldec-1-yl, 2-octyldodec-1-yl, or mixtures thereof; and wherein R¹, R² and R³ are each, independently, hydrogen, methyl, or an unsubstituted or substituted alkyl group containing 2 to 18 carbon atoms and, optionally, containing heteroatoms.

62. The composition according to claim 60, wherein the reaction product is a polymer comprising one or more the following structures: 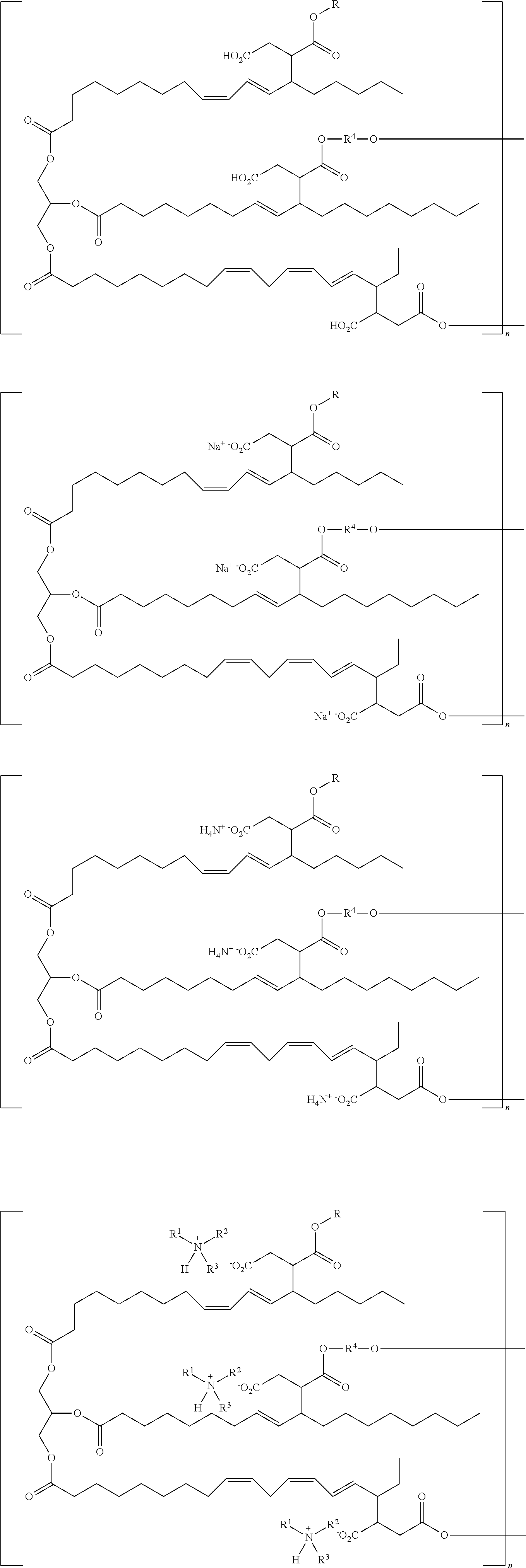
wherein 1<n<30; wherein R is ethyl, butyl, hexyl, octyl, 2-ethylhex-1-yl, 2-butyloct-1-yl, 2-hyxyldec-1-yl, 2-octyldodec-1-yl, or mixtures thereof; wherein R¹, R² and R³ are each, independently, hydrogen, methyl, or an unsubstituted or substituted alkyl group containing 2 to 18 carbon atoms and, optionally, containing heteroatoms; and wherein R⁴ is 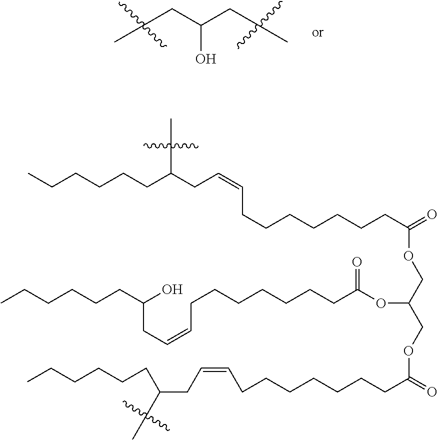 [SMILES: *CC(O)C* ; *C(C/C=C\CCCCCCCC(=O)OCC(COC(=O)CCCCCCC/C=C\CC(*)CCCCCC)OC(=O)CCCCCCC/C=C\CC(O)CCCCCC)CCCCCC]
or a mixture thereof.

63. The composition according to claim 60, wherein the reaction product is a polymer having a weight-average molecular weight from about 3,000 Daltons to about 20,000 Daltons and a weight-average degree of polymerization, n, of about 3 to about 20.

64. The composition according to claim 60, wherein the reaction product is a polymer having a viscosity measured at 25° C. with a Brookfield viscometer ranging from about 100 to 10,000 cps.

65. The composition according to claim 58, wherein the hydrophobic moiety is a moiety selected from those of the group consisting of unsubstituted or substituted alkyl, cycloalkyl, alkenyl, and aryl alcohols and amines, wherein the alcohols and amines contain from about 6 to about 36 carbon atoms and may contain heteroatoms; unsubstituted or substituted polyols, with or without heteroatoms, containing about 37 to about 60 carbon atoms; silicon-based compounds; and combinations thereof.

66. The composition according to claim 58, wherein the hydrophilic moiety is a moiety selected from those of the group consisting of unsubstituted or substituted alkyl, cycloalkyl, alkenyl, and aryl alcohols and amines, wherein the alcohols and amines contain from about 1 to about 5 carbon atoms and may contain heteroatoms; unsubstituted or substituted polyols, wherein the unsubstituted or substituted polyols contain from about 2 to about 36 carbon atoms and may contain heteroatoms; poly(ethylene glycol) monomethyl ethers (mPEGs) containing from 5 to 45 carbon atoms; silanes; and combinations thereof.

67. The composition according to claim 66, wherein the silane is functionalized with an alcohol, an amine, or combinations thereof.

68. The composition according to claim 65, wherein the hydrophobic moiety is that of an alcohol selected from the group consisting of hexanol, heptanol, nonanol, decanol, dodecanol, phenol, ethylbenzyl alcohol, 2-ethyl-1-hexanol, 1-octanol, 2-octanol, 2-butyl-1-octanol, 2-hexyl-1-decanol, 2-octyl-1-dodecyl alcohol, 2-tetradecanol, 2-hexadecanol, 3,7-dimethyl-1-octanol, 2-propyl-1-pentanol, 4-methyl-1-pentanol, and mixtures thereof.

69. The composition according to claim 66, wherein the hydrophilic moiety is that of an alcohol selected from the group consisting of methanol, ethanol, propanol, isopropanol, butanol, and mixtures thereof.

70. The composition according to claim 66, wherein the hydrophilic moiety is that of a polyol selected from the group consisting of ethylene glycol, propylene glycol, diethylene glycol, dipropylene glycol, dibutylene glycol, polyethylene glycol, polypropylene glycol, hexylene glycol, sorbitol, neopentylglycol, eythritol, mannitol, xylitol, threitol, pentaerythritol, beta-cyclodextrin, ribose, 2-deoxy-D-galactose, D-glucosamine, D-mannosamine, D-galactosamine, N-methyl-D-glucosamine, and mixtures thereof.

71. The composition according to claim 65, wherein the hydrophobic moiety is that of an amine selected from the group consisting of benzylamine, cyclohexylamine, hexylamine, methylhexylamine, phenethylamine, octylamine, oleylamine, decylamine, dodecylamine, hexadecylamine, octadecylamine, undecylamine, pentadecylamine, and mixtures thereof.

72. The composition according to claim 66, wherein the hydrophilic moiety is that of an amine selected from the group consisting of 2-methylpentane-1,5-diamine, diethanolamine, serinol, 2-amino-2-ethyl-1,3-propanediol, dimethylamine, 2-methylbutylamine, 3-amino-1-propanol, their hydrochloride salts, their ammonium salts, and mixtures thereof.

73. The composition according to claim 65, wherein the silicon based compound is a hydrophobic compound selected from the group consisting of aminopropylmethylsiloxane-dimethylsiloxane, N-ethylaminoisobutyl terminated polydimethylsiloxane, poly(1,1-dimethylsilazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine]terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

74. The composition according to claim 66, wherein the silane is a hydrophilic compound selected from the group consisting of 3-aminopropylsilanetriol, N-(2-aminoethyl)-3-aminopropylsilanetriol, and mixtures thereof.

75. The composition according to claim 58, wherein the base is selected from the group consisting of inorganic bases, organic bases, and mixtures thereof.

76. The composition according to claim 75, wherein the inorganic base is selected from the group consisting of oxides of alkali metals and alkaline earth metals, hydroxides of alkali metals and alkaline earth metals, carbonates of alkali metals and alkaline earth metals, bicarbonates of alkali metals and alkaline earth metals, oxides of transition metals, hydroxides of transition metals, carbonates of transition metals, bicarbonates of transition metals, and combinations thereof.

77. The composition according to claim 76, wherein the alkali metal is selected from the group consisting of lithium, sodium, potassium, rubidium, cesium, francium, and mixtures thereof.

78. The composition according to claim 76, wherein the alkaline earth metal is selected from the group consisting of beryllium, magnesium, calcium, strontium, barium, radium, and mixtures thereof.

79. The composition according to claim 76, wherein the transition metal is selected from the group consisting of scandium, titanium, vanadium, chromium, manganese, iron, cobalt, nickel, copper, zinc, yttrium zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, darmstadtium, roentgenium, and mixtures thereof.

80. The composition according to claim 76, wherein the inorganic base is selected from the group consisting of oxides, hydroxides, carbonates and bicarbonates of sodium, calcium, and combinations thereof.

81. The composition according to claim 58, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, maleated chickpea oils, maleated clary sage oils, and mixtures thereof.

82. The composition according to claim 58, wherein the maleated natural oil is a maleated soybean oil.

83. The composition according to claim 58, wherein the composition is selected from the group consisting of skin care compositions, oral care compositions, hair care compositions, energy compositions, lithium-ion battery compositions, construction compositions, biocidal compositions, preservative compositions, nutraceutical compositions, food compositions, agricultural compositions, coating compositions, ink compositions, cosmetic compositions, homecare compositions, industrial and institutional compositions, metal working compositions, textile compositions, laundry compositions, cleaning compositions, and disinfecting compositions.

84. An agricultural composition comprising:
a reaction product of
(A) a maleated natural oil, comprising a natural oil with maleated functionality;
(B) a functionalized or unfunctionalized moiety selected from the group consisting of hydrophobic moieties, hydrophilic moieties, and combinations thereof, wherein the maleated functionality has been partially reacted with the functionalized or unfunctionalized moiety; and
(C) a base.

85. The agricultural composition according to claim 84, wherein the reaction product comprises a compound of the following structure: 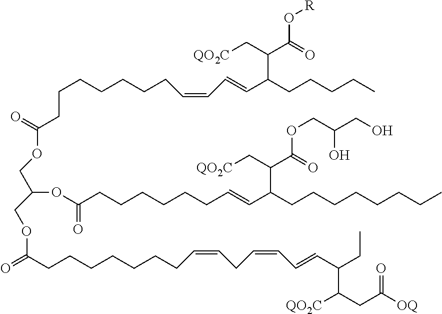
wherein R is one or more hydrophilic or hydrophobic moiety selected from the group consisting of unsubstituted or substituted alkyl, cycloalkyl, alkenyl, aryl and alkaryl moieties containing from about 1 to about 36 carbon atoms; and
wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

86. The agricultural composition according to claim 84, wherein the reaction product is a polymer comprising the following structure: 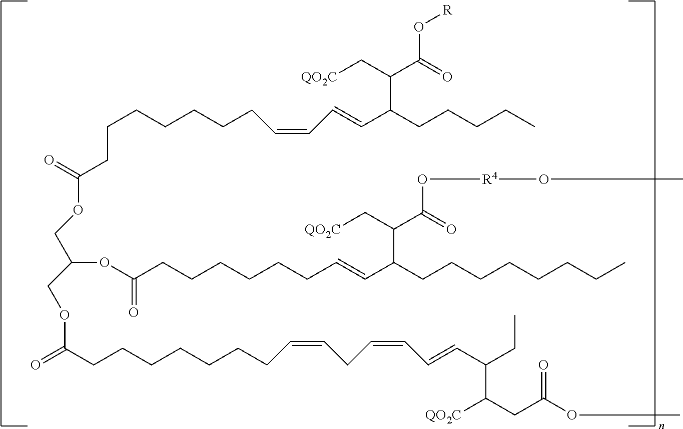
wherein n is greater than 1;
wherein R is one or more hydrophilic or hydrophobic moiety selected from the group consisting of unsubstituted or substituted alkyl, cycloalkyl, alkenyl, aryl and alkaryl moieties, with or without heteroatoms, containing from about 1 to about 36 carbon atoms; wherein R⁴ is one or more hydrophilic or hydrophobic moiety selected from the group consisting of unsubstituted or substituted alkylene, cycloalkylene, alkenylene, arylene and alkarylene moieties, with or without heteroatoms, containing from about 2 to about 60 carbon atoms; and
wherein each Q is independently selected from the group consisting of H, Li, Na, K, Rb, Cs, Fr, ½ Mg, ½ Ca, ½ Co, ½ Cu, ½ Zn, ammonium, alkylammonium, dialkylammonium, trialkylammonium and tetraalkylammonium.

87. The agricultural composition according to claim 85, wherein the reaction product comprises one or more structure selected from the group of the following structures: 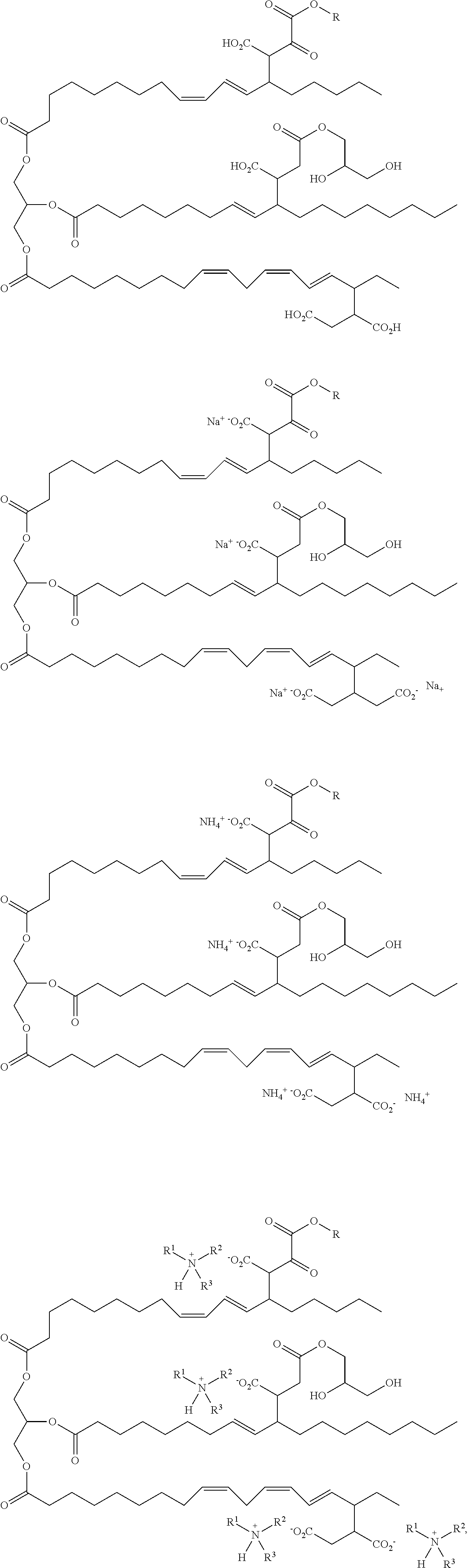
wherein R is ethyl, butyl, hexyl, octyl, 2-ethylhex-1-yl, 2-butyloct-1-yl, 2-hyxyldec-1-yl, 2-octyldodec-1-yl, or mixtures thereof; and wherein R¹, R² and R³ are each, independently, hydrogen, methyl, or an unsubstituted or substituted alkyl group containing 2 to 18 carbon atoms and, optionally, containing heteroatoms.

88. The agricultural composition according to claim 86, wherein the reaction product is a polymer comprising one or more the following structures: 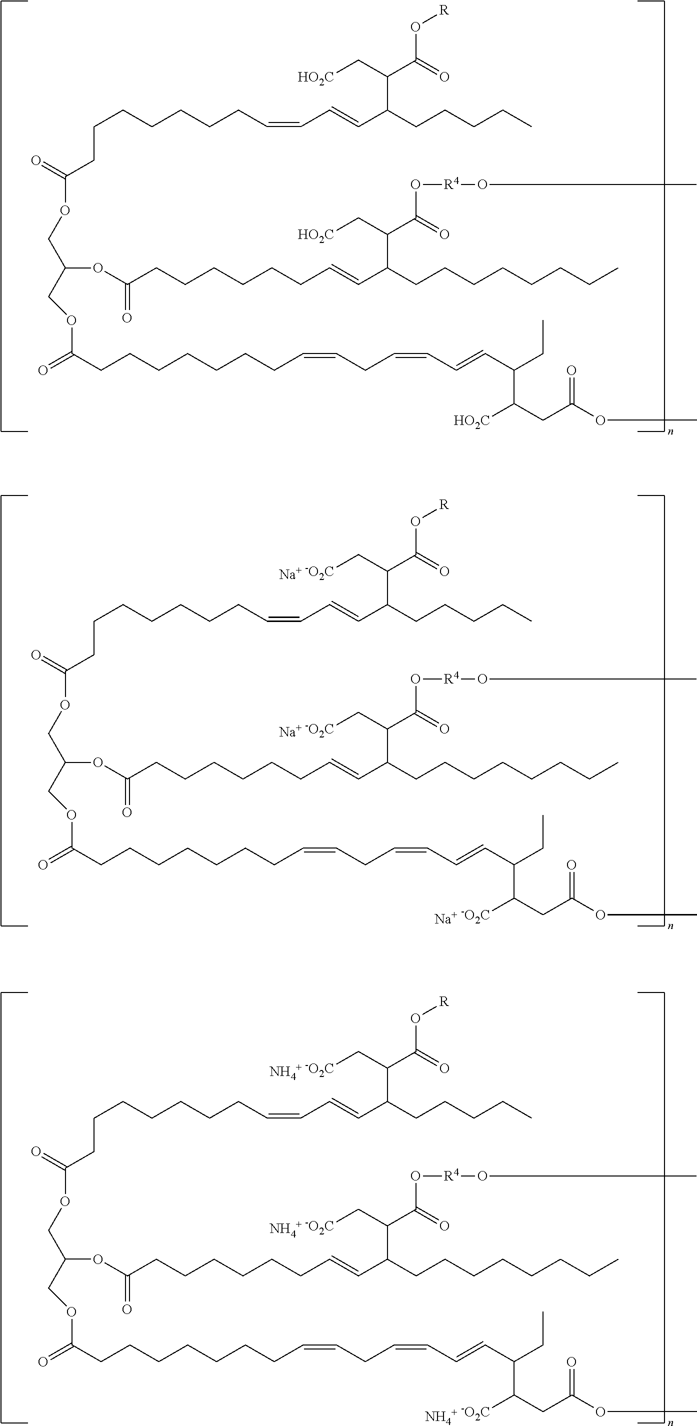 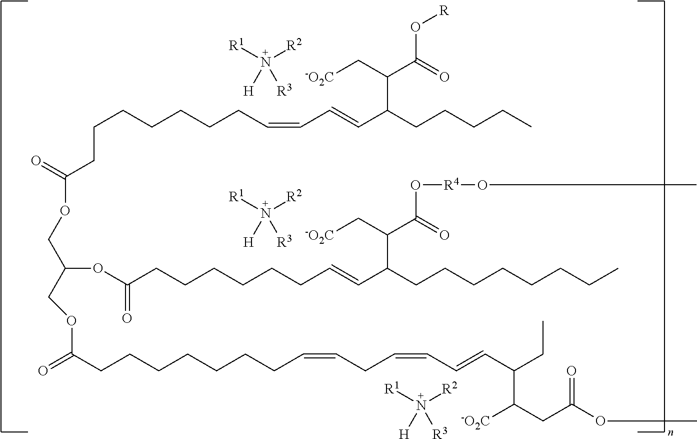
wherein 1<n<30; wherein R is ethyl, butyl, hexyl, octyl, 2-ethylhex-1-yl, 2-butyloct-1-yl, 2-hyxyldec-1-yl, 2-octyldodec-1-yl, or mixtures thereof; wherein R¹, R² and R³ are each, independently, hydrogen, methyl, or an unsubstituted or substituted alkyl group containing 2 to 18 carbon atoms and, optionally, containing heteroatoms; and wherein R⁴ is 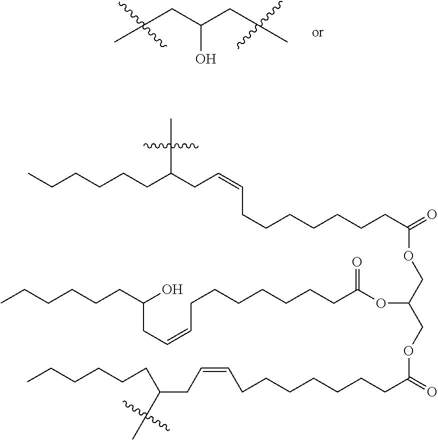 [SMILES: *CC(O)C* ; *C(C/C=C\CCCCCCCC(=O)OCC(COC(=O)CCCCCCC/C=C\CC(*)CCCCCC)OC(=O)CCCCCCC/C=C\CC(O)CCCCCC)CCCCCC]
or a mixture thereof.

89. The agricultural composition according to claim 86, wherein the reaction product is a polymer having a weight-average molecular weight from about 3,000 Daltons to about 20,000 Daltons and a weight-average degree of polymerization, n, of about 3 to about 20.

90. The agricultural composition according to claim 86, wherein the reaction product is a polymer having a viscosity measured at 25° C. with a Brookfield viscometer ranging from about 100 to 10,000 cps.

91. The agricultural composition according to claim 84, wherein the hydrophobic moiety is a moiety selected from those of the group consisting of unsubstituted or substituted alkyl, cycloalkyl, alkenyl, and aryl alcohols and amines, wherein the alcohols and amines contain from about 6 to about 36 carbon atoms and may contain heteroatoms; unsubstituted or substituted polyols, with or without heteroatoms, containing about 37 to about 60 carbon atoms; silicon-based compounds; and combinations thereof.

92. The agricultural composition according to claim 84, wherein the hydrophilic moiety is a moiety selected from those of the group consisting of unsubstituted or substituted alkyl, cycloalkyl, alkenyl, and aryl alcohols and amines, wherein the alcohols and amines contain from about 1 to about 5 carbon atoms and may contain heteroatoms; unsubstituted or substituted polyols, wherein the unsubstituted or substituted polyols contain from about 2 to about 36 carbon atoms and may contain heteroatoms; poly(ethylene glycol) monomethyl ethers (mPEGs) containing from 5 to 45 carbon atoms; silanes; and combinations thereof.

93. The agricultural composition according to claim 92, wherein the silane is functionalized with an alcohol, an amine, or combinations thereof.

94. The agricultural composition according to claim 91, wherein the hydrophobic moiety is that of an alcohol selected from the group consisting of hexanol, heptanol, nonanol, decanol, dodecanol, phenol, ethylbenzyl alcohol, 2-ethyl-1-hexanol, 1-octanol, 2-octanol, 2-butyl-1-octanol, 2-hexyl-1-decanol, 2-octyl-1-dodecyl alcohol, 2-tetradecanol, 2-hexadecanol, 3,7-dimethyl-1-octanol, 2-propyl-1-pentanol, 4-methyl-1-pentanol, and mixtures thereof.

95. The agricultural composition according to claim 92, wherein the hydrophilic moiety is that of an alcohol selected from the group consisting of methanol, ethanol, propanol, isopropanol, butanol, and mixtures thereof.

96. The agricultural composition according to claim 92, wherein the hydrophilic moiety is that of a polyol selected from the group consisting of ethylene glycol, propylene glycol, diethylene glycol, dipropylene glycol, dibutylene glycol, polyethylene glycol, polypropylene glycol, hexylene glycol, sorbitol, neopentylglycol, eythritol, mannitol, xylitol, threitol, pentaerythritol, beta-cyclodextrin, ribose, 2-deoxy-D-galactose, D-glucosamine, D-mannosamine, D-galactosamine, N-methyl-D-glucosamine, and mixtures thereof.

97. The agricultural composition according to claim 91, wherein the hydrophobic moiety is that of an amine selected from the group consisting of benzylamine, cyclohexylamine, hexylamine, methylhexylamine, phenethylamine, octylamine, oleylamine, decylamine, dodecylamine, hexadecylamine, octadecylamine, undecylamine, pentadecylamine, and mixtures thereof.

98. The agricultural composition according to claim 92, wherein the hydrophilic moiety is that of an amine selected from the group consisting of 2-methylpentane-1,5-diamine, diethanolamine, serinol, 2-amino-2-ethyl-1,3-propanediol, dimethylamine, 2-methylbutylamine, 3-amino-1-propanol, their hydrochloride salts, their ammonium salts, and mixtures thereof.

99. The agricultural composition according to claim 91, wherein the silicon based compound is a hydrophobic compound selected from the group consisting of aminopropylmethylsiloxane-dimethylsiloxane, N-ethylaminoisobutyl terminated polydimethylsiloxane, poly(1,1-dimethylsilazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine]terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

100. The agricultural composition according to claim 92, wherein the silane is a hydrophilic compound selected from the group consisting of 3-aminopropylsilanetriol, N-(2-aminoethyl)-3-aminopropylsilanetriol, and mixtures thereof.

101. The agricultural composition according to claim 84, wherein the base is selected from the group consisting of inorganic bases, organic bases, and mixtures thereof.

102. The agricultural composition according to claim 101, wherein the inorganic base is selected from the group consisting of oxides of alkali metals and alkaline earth metals, hydroxides of alkali metals and alkaline earth metals, carbonates of alkali metals and alkaline earth metals, bicarbonates of alkali metals and alkaline earth metals, oxides of transition metals, hydroxides of transition metals, carbonates of transition metals, bicarbonates of transition metals, and combinations thereof.

103. The agricultural composition according to claim 101, wherein the organic base is selected from the group consisting of ammonia, primary amines, secondary amines, pyridine, imidazole, benzimidazole, histidine, guanidine, and mixtures thereof.

104. The agricultural composition according to claim 102, wherein the alkali metal is selected from the group consisting of lithium, sodium, potassium, rubidium, cesium, francium, and mixtures thereof.

105. The agricultural composition according to claim 102, wherein the alkaline earth metal is selected from the group consisting of beryllium, magnesium, calcium, strontium, barium, radium, and mixtures thereof.

106. The agricultural composition according to claim 102, wherein the transition metal is selected from the group consisting of scandium, titanium, vanadium, chromium, manganese, iron, cobalt, nickel, copper, zinc, yttrium zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, darmstadtium, roentgenium, and mixtures thereof.

107. The agricultural composition according to claim 102, wherein the inorganic base is selected from the group consisting of oxides, hydroxides, carbonates and bicarbonates of sodium, calcium, and combinations thereof.

108. The agricultural composition according to claim 84, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, maleated chickpea oils, maleated clary sage oils, and mixtures thereof.

109. The agricultural composition according to claim 84, wherein the maleated natural oil is a maleated soybean oil.

110. The agricultural composition according to claim 84 that further comprises one or more agriculturally active ingredient and one or more additional ingredient.

111. The agricultural composition according to claim 110, wherein the agriculturally active ingredient is selected from the group consisting of fertilizers and pesticides selected from the group consisting of rodenticides, miticides, algicides, molluscicides, acaricides, avicides, insecticides, herbicides, ovicides, fungicides, germicides, antibiotics, antibacterials, antivirals, antifungals, antiprotozoals, anti-parasites and antimicrobials.

112. The agricultural composition according to claim 110, wherein the additional ingredient is an adjuvant or an inert ingredient.

113. The agricultural composition according to claim 112, wherein the adjuvant is selected from the group consisting of acidifying agents, buffering agents, anti-foam agents, defoaming agents, anti-transpirants, dyes and brighteners, compatibility agents, crop oil concentrates, oil surfactants, deposition agents, drift reduction agents, foam markers, feeding stimulants, herbicide safeners, spreaders, extenders, adhesive agents, suspension agents, gelling agents, synergists, wetting agents, emulsifiers, dispersing agents, penetrants, tank and equipment cleaners, neutralizers, water absorbents, water softeners and mixtures thereof.

114. The agricultural composition according to claim 112, wherein the additional ingredient is selected from the group consisting of solvents, liquid carriers, solid carriers or fillers, surfactants, solubilizers, penetration enhancers, protective colloids, thickeners, humectants, repellents, attractants, compatibilizers, anti-freezing agents, crystallization inhibitors, colorants, tackifiers, binders, preservatives, pH adjuster, clarifiers, stabilizers, UV stabilizers, and mixtures thereof.

115. The agricultural composition according to claim 84, where in the agricultural composition is an adjuvant composition, a fertilizer composition, a nutrient composition, a plant strengthener composition, a seed coating composition, a soil conditioner composition, a livestock composition, a granular composition, a controlled release composition, a film coating composition, a pesticide composition selected from the group consisting of ovicide, rodenticide, insecticide, miticide, algicide, molluscicide, acaricide, avicide, fungicide and herbicide compositions, a germicide composition, an antibiotic composition, an antibacterial composition, an antiviral composition, an antifungal composition, an antiprotozoal composition, an anti-parasite composition, a wood preservation composition, an antimicrobial composition or a yield enhancer composition to soil for the plant.

116. The agricultural composition according to claim 84, wherein the agricultural composition is in the form of an aqueous or a non-aqueous composition comprising a capsule suspension, an emulsifiable concentrate, an emulsion for seed treatment, a concentrated aqueous emulsion, a microemulsion, a suspoemulsion, an oil-in-water emulsion, a flowable concentrate for seed treatment, an oil dispersion, a suspension concentrate, a water dispersible granule or a wettable powder.

117. The agricultural composition according to claim 84, wherein the agricultural composition is formulated as a liquid, a gel, a paste, a powder, a granule, a tablet, an emulsion, or a pellet.

118. An agricultural composition for topical application to an animal or a plant that comprises a safe and effective amount of
(A) from about 0.01 to about 20.0 wt. % of the reaction product of claim 84;
(B) from about 1.0 to about 90.0 wt. % of one or more agriculturally active ingredient; and
(C) from about 1.0 to about 99.0 wt. % of one or more additional ingredient.

119. A method for treating vegetation comprising applying an agricultural composition of claim 84 to a plant, plant foliage, blossoms, leaves, stems, fruits, the area adjacent to a plant, soil, seeds, germinating seeds, roots, liquid and solid growth media, flower, bud, or hydroponic growth solutions.

120. A method of treating an animal comprising topically applying an agricultural composition of claim 84 to an area of skin of an animal.
